(19) United States
(12) Patent Application Publication
(10) Pub. No.: US 2020/0393376 A1
(43) Pub. Date: Dec. 17, 2020

(54) System, Device, and Method for Identifying and Monitoring Breast Milk Composition

(71) Applicant: Mao Foodtech, Ltd., Qiriat Ata (IL)

(72) Inventors: Ariel Orbach, Jerusalem (IL); Ashkenazi Maya, Qiriat Ata (IL); Yuval Appelbaum, 3709018 Pardes Hanna-Karkur (IL)

(73) Assignee: Mao Foodtech, Ltd., Qiriat Ata (IL)

(21) Appl. No.: 16/977,787

(22) PCT Filed: Feb. 7, 2019
(86) PCT No.: PCT/IL2019/050158

§ 371 Date: Sep. 2, 2020

Related U.S. Application Data
(60) Provisional application No. 62/638,908, filed on Mar. 5, 2018.

(51) Int. Cl. G01N 21/64; G01N 21/78; G01N 33/50; G16H 10/40

(52) U.S. Cl. CPC G01N 21/64; G01N 21/78; G01N 2333/70589; G16H 10/40; G01N 33/50

(57) ABSTRACT

There is provided herein a device for the analysis of breastmilk, comprising: at least one camera to photograph a sampling element, wherein said sampling element comprises one or more arrays of reagents reacting with one or more target molecules present in a sample of said breastmilk and presenting one or more arrays with a plurality of spots comprising one or more intensity levels, and wherein said camera is adapted to photograph said plurality of spots; at least one computer adapted to analyze said one or more intensity levels of said spots; and at least one output component for presenting results of said analysis and for providing a recommendation relating to nutritional or immunological needs of an infant fed by said breastmilk.

### FIELD OF THE INVENTION

The present invention relates to the field of nutrition and immunology, specifically to the detection and/or monitoring of specific elements within maternal breast milk.

### BACKGROUND OF THE INVENTION

Breast milk is the milk produced by the breasts (or mammary glands) of a human female to feed a child. About 40% of infants are exclusively breastfed, while over 50% of them are fed by a combination of breast milk and milk substitutes.

The various health benefits of breastfeeding have long been known. The most prominent of these are the nutritional and immunological aspect. Milk is the primary source of nutrition for newborns before they are able to consume and digest other foods; older infants and toddlers may continue to be breastfed, either exclusively or in combination with other foods from around six months of age when solid foods may be introduced. Additionally, breast milk is an essential source of immunoglobulins (i.e. antibodies), which are proteins found in the blood and function as immune defenses against infectious agents, such as viruses and bacteria. Some types of these antibodies (mainly sIgA whose function is to protect from pathogen invasion through mucosal tissues) are transferred from the plasma or the mother's blood into breast milk, or are locally produced in the mammary glands by cells that have migrated to the area, and form the primary immune defense mechanism of the nursing infant.

When breastfeeding is not possible or not desired, infant formula may be provided. Infant formula is a manufactured food designed and marketed for feeding to babies and infants, usually prepared for bottle-feeding or cup-feeding from powder (mixed with water) or liquid (with or without additional water).

Today the formulas are based on stages and the babies are moving from one stage to another according to their age. 1-6, 6-12 months and above. These stages are defined according to an average with no specific measurement of the baby's individual need.

Monitoring the baby's development is done according to growing curves and specific tests (e.g. blood tests) that are done in cases of abnormalities.

However, different babies have different nutritional requirements while providing a general formula based on average needs often fails to meet the specific requirements of an individual infant. In addition, certain diseases in the lactating mother may be transferred to her baby by the milk he consumes. In many cases, these are detected only after the baby develops the disease himself. Therefore, early identification and monitoring of components in breast milk that suggest or increase the risk of morbidity in the infant may have an important prognostic value.

Previous findings have described methods for analyzing the nutritional composition of breast milk, while others have described methods for enriching breast milk with essential nutrients. Bellmonte et al. (Ann. 1st. Super. Sanita, Vol 26, N. 2 (1990), pp. 131-140) describes the protein and lipid consumption of human milk and infant formulas.

WO 2008/111942 discloses a method of tailoring infant formulas to individual nutritional needs prior to use.

US20080124430 teaches a human milk fortifier compositions, standardized human milk, and methods of making and using same. In one embodiment, a pasteurized human milk composition includes a human protein constituent of about 35-85 mg/mL; a human fat constituent of about 60-110 mg/mL; and a human carbohydrate constituent of about 60-140 mg/ml.

U.S. Pat. No. 4,692,340 teaches a procedure for the production of a granulated infant milk food product. US20080187619 relates to a human milk fortifier as well as to several uses and a method for the production of such a fortifier. A particularly beneficial fortifier can be realized in that at least one human component based on a product directly or indirectly derived from human mammary secretion during non-pregnant, pregnant, lactating and/or involuting periods is used, giving rise to an optimally adapted fortifying effect which is particularly useful in the context of feeding preterm infants.

US20080118615 relates to a method for analyzing and treating human milk to be fed to an infant comprising the steps of collecting own mother's milk, taking a sample of the collected own mother's milk, conducting nutritional analysis on said sample, using the collected own mother's milk as nutrition for the infant and using the collected own mother's milk in the form of at least one of the group of: unchanged own mother's milk, fortified own mother's milk, unchanged components of own mother's milk and fortified components of own mother's milk, wherein said form is chosen depending on at least some of said results of the analysis and said infant's condition, the infants condition being chosen from at least one of the following parameters: infant's age, infant's weight, infant's health, infant's shortcomings, infant's deficiencies, time of day when the milk is fed to the infant.

In the immunological aspect, other previous findings have described methods for analyzing breast milk, and more specifically antigen and antibody levels in breast milk, but none of them has evaluated the relationship between these levels and the risk of morbidity in the nursing infant:

Hassiotou et al. (Clinical & Translational Immunology (2013) 2, e3; doi:10.1038/cti.2013.1) examined the influence of an infectious disease in usually healthy mother/infant dyads on breast milk leukocytes and immunomodulatory biomolecules compared to the baseline range of these components. Under infection state, leukocyte numbers significantly increased up to 94% leukocytes out of total cells, as compared to a baseline level in mature breastmilk of 0-2%. Upon recovery from the infection, baseline values were restored. Exclusive breastfeeding was associated with a greater baseline level of leukocytes in mature breastmilk. The authors concluded that their results suggest a strong association between the health status of the mother/infant dyad and breastmilk leukocyte levels. This could be used as a diagnostic tool for assessment of the health status of the lactating breast as well as the breastfeeding mother and infant.

Hassiotou et al. (American Society for Nutrition. Adv. Nutr. 6: 267-275, 2015) further review the effect of an infection on breast milk composition, mainly leukocytes concentrations which consistently and rapidly increase, returning to baseline concentrations upon recovery.

Field et al. (J Nutr. 2005 January; 135(1):1-40) reviews the immunological components found in breast milk. They state that depending on the phase and stage of lactation, a variety of leukocytes are present in colostrum (4×10⁹/L) and mature milk (10⁸-10⁹/L) that might play a role in promoting the development of the neonatal immune response. Macrophages (55-60%) and neutrophils (30-40%) dominate over lymphocytes (5-10%).

Breakey et al. (Evolution, Medicine, and Public Health [2015] pp. 21-31) examined the relationship between the presence of immune compounds in human milk and infant health. According to one paradigm, elevated levels of such compounds suggest a protective effect to the infant, while an opposite paradigm suggests that elevated levels of such compounds in breast milk merely implies a response to infection. Milk samples and illness data were collected from 30 mother-infant dyads. Samples were assayed for two immune proteins, lactoferrin and secretory immunoglobulin A (sIgA). Generalized estimating equations were used to assess the relationship between immune composition of milk and symptoms of illness in infants. The results showed opposite relationship for the two markers (Lactoferrin was positively associated with symptoms of illness in infants while sIgA's association with such symptoms was negative).

WO2009033011A1 describes a method for measuring the level of at least one secretor antigen in a biological sample, by which it enables identifying individuals at risk for inflammatory or other type of disease (e.g. necrotizing enterocolitis). It mentions the use of secretor, lewis and sialyl antigen levels as predictors for disease. In one embodiment, the biologic fluid being tested is breast milk, while ELISA or chromatography are possible methods for such analysis. WO2011127219A1 discloses methods and systems for characterizing a phenotype by detecting biomarkers that are indicative of disease or disease progress. The biomarkers can be circulating, including vesicles and/or microRNAs. In one embodiment, said vesicles may be found in breast milk which may serve as the biological sample. However, these findings do not teach about any possible implication to an infant being exposed to said biological sample, more specifically to breast milk.

US20030065277A1 describes method and apparatus for detecting a biological factor in a fluid sample obtained from a mammary gland, comprising the steps of: non-invasively obtaining a mammary gland fluid from a subject comprising warming the mammary gland; massaging the mammary gland; extracting the mammary fluid from the nipple by expression or aspiration; and detecting the biological factor in the mammary gland fluid. More specifically, it provides method for determining the risk of breast cancer based on breast milk composition. In this context, biological factors may include active cells, proteins, chemicals, lipids, growth factors, cytokines, nucleic acid molecules.

U.S. Pat. No. 5,798,266A discloses a non-invasive methods and kits for obtaining biological samples of mammary fluid or mammary fluid components. It describes the method of stimulating breast fluid expression, collecting it in the form of whole mammary fluid, whole cells or cellular components, other selected liquid or solid fractions of the mammary fluid, purified or bulk proteins, glycoproteins, peptides, nucleotides or other desired constituents of mammary fluid.

Methods and kits are also provided for determining the presence or amount of a breast disease marker, specifically breast cancer. Among the possible breast cancer markers are included CEA, HIVIFG, MCA, vasopressin, or cathepsin D. the kit includes oxytocin for intranasal administration, breast pump and immunological probes specifically designed to bind to and detect breast disease markers.

Similarly, U.S. Pat. No. 6,471,660B1 also describes a method for analyzing breast milk components for determining a risk of breast disease, more specifically risk for developing breast cancer in breastfeeding women.

CN 106226390 A describes a breast milk composition analyzer, which includes a sampling device; a temperature detection device for detecting temperature of a sample in-sucked by a sample mechanism; a detection device which uses ultrasonic detecting means for detecting composition of the sample; a cleaning device; an output device for outputting structures detected by the detection device; and a control device which controls a sample suction pump. CN104880566 A also discloses a breast milk analyzer, which comprises a sampling unit, an analyzing unit, a waste breast milk container and cleaning units. The sampling unit is connected with the analyzing unit via a first pipeline, the analyzing unit is in one-way connection with the waste breast milk container via a second pipeline and a waste breast milk pump, each of the cleaning units comprises a cleaning container, a cleaning pipeline and a cleaning pump, and each cleaning container is in one-way connection with the first pipeline via the corresponding cleaning pipeline and the corresponding cleaning pump. The invention further discloses a using method and an automatic cleaning method of the breast milk analyzer. US20140155281 discloses a method for assessing the infection status of a subject and in particular to a method for assessing the infection status of a subject by analyzing the cellular and/or humoral composition of breastmilk from said subject. The invention aims for detecting infection in a breastfeeding mother and/or infant. It includes analyzing breast milk composition of immunological markers, and further comparing said composition with a reference, wherein an increase in the proportion of said markers when compared to said reference indicates that said mother and/or said infant have an infection. immunological marker is selected from immune cells (IC), cytokines, and immunoglobulins.

### SUMMARY OF THE INVENTION

According to some demonstrative embodiments, there is provided herein a system, device and method for monitoring an infant's development and predicting his likelihood for acquiring an infectious disease.

According to some embodiments, monitoring the development of the infant may include analyzing the maternal milk and accordingly adapting the proper formula for the infant based on his specific needs. For example, in cases wherein the mother is combining breastfeeding with the administration of an infant formula and/or a milk fortifier.

According to some embodiments, monitoring the development of the infant may include testing and/or analyzing the infant's individual parameters.

According to some embodiments, predicting infant's likelihood for acquiring an infectious disease may include analyzing the maternal milk and alerting against abnormal levels of milk components (i.e. antigens and/or antibodies).

According to some embodiments, there is provided herein a system for the analysis of the composition of a breastmilk, comprising at least one sampling element for sampling and analysis of said breastmilk; and an application for deciphering results of the analysis; wherein said analysis comprising a nutritional and immunological analysis.

According to some embodiments, the nutritional analysis may include a determination of the amount or concentration of macromolecules and nutrients in said breastmilk.

According to some embodiments, the immunological analysis may include determination of the amount or concentration of antibodies in the breastmilk.

According to some embodiments, the application may be installed upon a mobile device.

According to some embodiments, there is provided herein a system for determining the nutritional needs of an infant, comprising a collection and/or analysis device, also referred to herein as a “sampling element”, to collect a sample of a maternal milk from a mother of said infant and to analyze the milk to measure at least one parameter; and a result indicator to provide a result, for example, a result which corresponds to a specific infant nutritional or immunological needs, e.g., indicating a specific formula to be administered to the specific infant.

According to some embodiments, the sampling element may include a cartridge with a funnel or other sampling element.

According to some embodiments, the at least one parameter may be selected from the group including whole protein concentration, oligosaccharides concentration, pH measurement, fat concentration, number of cells, Properdin, Vitamins, e.g., Vitamin B, Phosphorus, Iron and the like.

According to some embodiments, the at least one parameter may be whole protein concentration.

According to some embodiments, the result may be selected from the group including number indicators, color indicators, marking indicators, Latin letter indicators, and/or any other suitable electronic indicators, e.g., sound, beeping and the like.

According to some embodiments, there is provided herein a method for establishing one or more nutritional needs of an infant, wherein the method may include measuring at least one parameter in one or both of: a maternal milk and/or a blood sample of said infant and/or a saliva sample of said infant; determining said one or more nutritional needs of said infant based on said at least one parameter; and indicating an optimal nutritional infant product based on said one or more nutritional needs of said infant.

According to some embodiments, there is provided herein a device for establishing one or more nutritional needs of an infant, wherein the device may be adapted to analyze a sample of maternal milk from a mother of an infant (e.g., via a sampling element), wherein the device may also provide a recommendation of a specific nutritional formula for the infant based on the at least one parameter, and/or provide a mother with nutritional recommendations to consume more or less of a specific nutrient.

According to some embodiments, the device may analyze the sampling element using photographing the element. According to these embodiments, the sampling element may include one or more reagents which cause one or more reactions with specific components in the analyzed breastmilk.

According to these embodiments, the device may include at least one camera to take a photograph of the sampling element after the reactions have occurred.

According to some embodiments, the camera may be a monochromatic camera, for example, capable of taking black and white pictures.

According to some embodiments, the device may include one or more computers adapted to analyze the pictures of the sampling element using, for example, a predetermined algorithm.

According to some embodiments, as per the algorithm the device may calculate an average of the color as resulting from the one or more reactions in the array. According to some embodiments, the term “Intensity of the color” (Also referred to herein as “intensity of grey”) may relate to a scale of infinite number ranging from 0 to 1, wherein 0 represents white and 1 represents black.

According to some embodiments, the device may assign a number on a scale from 0 to 1, which may be used to represent Optical density (“OD”).

According to some embodiments, the use of such a scale may enable extrapolation of the analysis results into a linear graph. This scale and subsequent linear graph extrapolated from it may provide an injective function, for example, making it easy to predict the concentrations of target molecules in the analyzed breastmilk sample (wherein each target molecule may have its own function).

According to some embodiments, the calculation results may be compared to various other results, also referred to herein as the “previous results”. The previous results may include the user's results from past analysis, to the average results of other users in the data bank, and to results described in the relevant literature (also referred to herein as “gold standard”).

According to some embodiments, if the results of an analysis indicate a concentration or amount of a target molecule which deviates from the gold standard, the device may indicate various courses of action, such as, consulting a medical professional, consulting with a breastfeeding expert, making dietary changes, referring to a specific supplement, offering a personalized supplement, etc.

If the result for a specific target if on or above the wanted average, then the algorithm will simply show the result on a scale and will not offer any further steps. The algorithm will be constantly “learning”: adding user information, incorporating new studies, changing recommendations for a specific user based on the user's feedbacks, etc.

Nutritional Factors and Gold Standard:

Each nutritional factor has a desired range of concentration in the breastmilk (obtained from literature or experiments). The algorithm will compare a specific result to this range—if the result is below the range, then the user will receive a notice with suggestions for possible courses of action (consulting with a nutritionist/breastfeeding consultant, taking supplements, dietary changes etc.). If the result is within or above the range, the user will only see their result on a scale, with the message telling her the result is on or above the standard (no further action needed).

Immune Factors and Gold Standard:

Some immune factors, such as general immunoglobulins, some cytokines, lactoferrin, etc. are always present in the breastmilk, and only when they pass a certain threshold it is possible to determine that an illness or ailment is developing. Other more specific immune factors, such as CMV-IgM, are only present in the breastmilk when either the infant or the mother has their corresponding disease, therefore the test must only show a “present/not present” result. The test for the different cluster of differentiations (e.g. CD-45) is usually performed with a cell count. Future targets for these factors will include: CD-3 CD-4, CD-8, CD-14, CD19 and CD-56 (CD-45 is already a target). At this point, it will be possible to determine the concentration of each of these factors by comparing signal densities between a single target and CD-3.

According to some embodiments, there is provided herein a use of the system of the present invention for the analysis of a breastmilk fed to an infant, comprising analyzing a sample of the breastmilk, determining the amount or concentration of one or more antibodies in the breastmilk and determining the chances of existence of a disease in the infant and/or in the breastfeeding mother.

According to some embodiments, there is provided herein a use of the system of the present invention for the analysis of a breastmilk fed to an infant, comprising analyzing a sample of the breastmilk, determining the amount or concentration of one or more target molecules (including for example macromolecules or nutrients) in the breastmilk and suggesting an optimal nutritional supplements and/or suitable supplemental formula to the infant and/or mother based on the analysis of the breastmilk.

According to some embodiments, there is provided herein a device which may perform an analysis of the sampled breastmilk contained upon the sampling element of the present invention.

According to some embodiments, the device may encompass a sample of breastmilk which may include at least one incubator to incubate the sample of the breastmilk.

According to some demonstrative embodiments, the device may be any suitable electronical device adapted to encompass the sampling element as taught herein or alternatively, be adapted to read one or more indications located on said sampling device, and provide an output, e.g., an analysis of the composition of a breastmilk sampled by the sampling element.

According to some embodiments, the device may be a smartphone, capable of reading one or more indications upon the sampling element, e.g., via the camera of the smartphone.

According to some embodiments, there is provided herein a sampling element for the sampling and analysis of breastmilk, wherein the element may include: a plurality of fibers to allow the flow of said breastmilk through the sampling element; a result indicator to indicate the result of the analysis; and wherein the sampling element may provide a nutritional or immunological analysis of said breastmilk.

According to some embodiments, the sampling element may include at least three separate zones, a first zone, a second zone and a third zone for analyzing the breastmilk, wherein the breastmilk flows through the at least three zones.

According to some embodiments, the at least three separate zones include: zone 1 comprises antibodies that bind to specific components within said breastmilk, and wherein the antibodies are conjugated to an enzyme that induces a color change; zone 2 comprises particles that induce the color release from conjugated enzymes and antibodies bound to specific components; and zone 3 comprises particles that induce the color release from conjugated enzymes and antibodies bound to specific components that did not induce a color release in zone 2.

According to some embodiments, the nutritional analysis may include a determination of the amount or concentration of macromolecules or nutrients in the breastmilk.

According to some embodiments, the macromolecules or nutrients are selected from the group including: Vitamin B, Human Milk Oligosaccharides (HMO), long chain polyunsaturated fatty acids (LCPUFA) and Total Proteins.

According to some embodiments, the immunological analysis may include the determination of the amount or concentration of at least one immunological factor in the breastmilk selected from the group including: Lactoferrin, Leukocytes and Immunoglobulins.

According to some embodiments, the at least one immunological factor may be Lymphocyte Common Antigen (CD45)

According to some embodiments, the at least one immunological factor may be sIgA.

According to some embodiments, the sampling element may be selected from the group including a test strip, a test stick, a dipstick or a vial.

According to some embodiments, the sampling element may be disposable. According to these embodiments, the use of disposable sampling elements may be beneficial since the user of the sampling elements are usually mothers and the test are preferably conducted on a daily basis or a couple of times a week.

According to some embodiments, the sampling element may be adapted to be inserted into a device for the analysis of said breastmilk.

According to some embodiments, there is provided herein a system for determining the nutritional needs of an infant, comprising: the sampling element to collect and analyze a sample of breastmilk from a mother of said infant and provide results of said analysis; an application to read said results and provide a recommendation for a specific infant formula to be fed to said infant based on said results; and wherein said sampling element analyzes at least one parameter selected from the group including whole protein concentration, separate peptides, a combination of peptides, oligosaccharides concentration, pH measurement, fat concentration, number of cells, Properdin, Vitamin A, Phosphorus and Iron.

According to some embodiments, the application may be installed upon a mobile device.

According to some embodiments, there is provided herein a use of the sampling element for determining the amount or concentration of one or more macromolecules or nutrients in said breastmilk and suggesting an optimal nutritional supplemental formula to said infant based on the analysis of said breastmilk.

According to some embodiments, there is provided herein a use of the sampling element for determining the amount or concentration of one or more immunological factor for alerting the development of an infectious disease in the infant or mother.

According to some demonstrative embodiments, there is provided herein a device for the analysis of breastmilk, comprising: at least one camera to photograph a sampling element, wherein the sampling element may comprise one or more arrays of reagents reacting with one or more target molecules present in a sample of the breastmilk and presenting one or more arrays with a plurality of spots comprising one or more intensity levels, and wherein the camera is adapted to photograph the plurality of spots; at least one computer that may be adapted to analyze the one or more intensity levels of the spots; and at least one output component for presenting results of the analysis and/or for providing a recommendation relating to nutritional or immunological needs of an infant fed by the breastmilk.

According to some embodiments, the camera may be a monochromatic camera.

According to some demonstrative embodiments, the device may include one or more lighting elements configured to specifically light one or more portions of the sampling elements. According to these embodiments, the one or more lighting elements may be positioned at a specific location to provide a more accurate photograph of the sampling element, e.g., in comparison to a photograph taken with the use of the lighting element. For example, as explained herein, the sampling element may inserted in and/or positioned onto the device, e.g., to be photographed in order to be analyzed. According to some embodiments, the lighting element may be positioned below the sampling element, e.g., to enhance the contrast between the spots and any blank area surrounding the spots. According to other embodiments, the lighting element may be positioned on top of, or with a predefined angel to the sampling element, e.g., to enhance the contrast between the spots and any blank area surrounding the spots.

According to some embodiments, the one or more lighting elements may include any suitable lamp or light and may be selected for example for a group including: spot lights, bar lights, flash, LED light and the like.

According to some embodiments, the computer may comprise a pre-defined algorithm for the comparison of the results with other data.

According to some embodiments, the other data may be selected from the group including: previous results obtained by the user of said device, gold standard values, average values or ranges of target molecules as presented in the relevant literature.

According to some embodiments, the recommendation may include one or more suggestions to change the diet of the infant.

According to some embodiments, the recommendation may include one or more indications regarding a status of infection or disease in the infant.

According to some embodiments, the analysis comprises a determination of the amount or concentration of macromolecules or nutrients in said breastmilk.

According to some embodiments, the macromolecules or nutrients may be selected from the group including: Vitamins, Human Milk Oligosaccharides (HMO), long chain polyunsaturated fatty acids (LCPUFA) and Total Proteins.

According to some embodiments, the analysis may comprise an immunological analysis comprising determination of the amount or concentration of at least one immunological factor in the breastmilk selected from the group including: Lactoferrin, Leukocytes and Immunoglobulins.

According to some embodiments, the at least one immunological factor is Lymphocyte Common Antigen (CD45).

According to some embodiments, the at least one immunological factor is sIgA.

According to some embodiments there is provided herein a system for the analysis of breastmilk, comprising the device described hereinabove and a sampling element comprising a lateral flow stick comprising a microarray of molecules selected from the group including antibodies, proteins and vitamins.

According to some demonstrative embodiments there is provided herein a use of the device of the present invention for providing at least one recommendation regarding dietary or immunological needs of an infant breast fed by said breastmilk.

### BRIEF DESCRIPTION OF THE DRAWINGS

FIGS. 1A to 1H are graphs depicting the changes in different components in various infant formulas during the various stages, in accordance with some demonstrative embodiments.

FIG. 2 is a graph depicting the change in Properdin in correlation to the age of the infant, in accordance with some demonstrative embodiments.

FIG. 3 is an exemplary scheme for assisting in the identification of a suitable nutritional formula for an infant, in accordance with some demonstrative embodiments.

FIG. 4 is an illustration of a sampling element (SE), and the movement therein of a sample liquid, according to some demonstrative embodiments.

FIGS. 5A to 5D are illustrations of a dipstick sampling element with a panel, according to some demonstrative embodiments.

FIG. 6 illustrates a horizontal stick flow sampling element, according to some demonstrative embodiments.

FIG. 7 depicts a flow chart of different possible options for sampling and analyzing breast milk using the system of the present invention, according to some demonstrative embodiments.

FIG. 8 illustrates possible uses for the system of the present invention in accordance with some demonstrative embodiments.

FIG. 9 is a schematic illustration of the system of the present invention and the components thereof, in accordance with some demonstrative embodiments.

FIG. 10 illustrates an algorithm of activities and actions of the system of the present invention, in accordance with some demonstrative embodiments.

FIG. 11 is a flow chart of the operation of the system of the present invention, in accordance with some demonstrative embodiments.

FIG. 12 is a graph depicting the protein concentrations of Nutrilon® infant formula in three phases of products, as indicated by the manufacturer.

FIG. 13 is a graph depicting the protein concentrations of the human milk at different age groups, using Bradford assay.

FIG. 14 is a graph depicting the average relative and normalized Glycoprotein-Carbohydrates Percentage at protein concentration 2.5 mg/ml. n=24 vs. age groups.

FIG. 15 is an illustration which shows how to prepare a series of dilutions, in accordance with some demonstrative embodiments disclosed herein.

FIG. 16 is a graph depicting the protein concentrations of the different formulas discussed in the present application using BCA and Bradford methods, in comparison to the manufacturer's values, in accordance with some embodiments (Error bars are of standard error. n=2-3.) FIG. 17 is a graph depicting the protein concentrations of breast milk at different age groups of a baby, using BCA and Bradford methods, in accordance with some embodiments (Error bars are of standard error. n=4-8).

FIG. 18 is a graph depicting the protein Concentration of breastmilk at the different infant age groups, results obtained by the Technion Proteins Lab using the Bradford Method (n=3-5).

FIG. 19 is a graph depicting the comparison of protein concentration by age group as discovered by the MAO FoodTech lab team and the Technion Proteins Lab (n=3-8).

FIG. 20 is a graph depicting the protein concentration results for each individual sample, organized by age [months, days], in accordance with some embodiments.

FIG. 21 is a graph depicting the average protein concentration results when age groups are broken down into the general manufacturer's definitions for each formula phase (i.e. phase one 0-6 months, phase 2 6-12 months etc.’.) FIG. 22 is a graph depicting the average Glycoprotein Carbohydrates Concentration of all three tested formulas at 2.5 mg of protein per 1 ml liquid.

FIG. 23 is a graph depicting the average Glycoprotein Carbohydrates Concentration of all three tested formulas at 0.25 mg of protein per 1 ml liquid.

FIG. 24 is a graph depicting the average Glycoprotein Carbohydrates Concentration for each of the six age groups at 2.5 mg of protein per 1 ml liquid (n=4-5).

FIG. 25 is a graph depicting the average Glycoprotein Carbohydrates Concentration for each of the six age groups at 0.25 mg of protein per 1 ml liquid (n=4-5).

FIGS. 26 and 27 are tables demonstrating various candidates that may be analyzed and/or monitored by the device of the present invention in accordance with some demonstrative embodiments.

FIG. 28 is an exemplary legend in accordance with some demonstrative embodiments.

FIG. 29 is an illustration of an exemplary legend, to which a sampling element result may be compared to, in accordance with some demonstrative embodiments.

FIG. 30 is an illustration of an exemplary legend of average concentrations of Vitamin B1 in the breastmilk of a mother feeding an infant in correlation to the age of the infant, in accordance with some demonstrative embodiments.

FIG. 30 is an illustration of an exemplary legend of average concentrations of Vitamin B12 in the breastmilk of a mother feeding an infant in correlation to the age of the infant, in accordance with some demonstrative embodiments.

FIG. 32 is an illustration of a sampling element for LC-PUFA with a predetermined marked line, to indicate to the user of the sampling element the depth up to which the user should dip the element into the tested breastmilk, in accordance with some demonstrative embodiments.

FIG. 33 is an illustration of an exemplary control panel, wherein sampling element is inserted into the panel, and the results indicated upon element can be compared to the reference results indicated upon the panel, in accordance with some demonstrative embodiments.

FIG. 34 is an illustration of a flow chart depicting the action of the device of the present invention in accordance with some demonstrative embodiments.

FIGS. 35A and 35B FIG. 35A depict exemplary photos of the results of various reactions taking place within sampling elements, according to some demonstrative embodiments.

FIG. 36 depicts a graph demonstrating the linear equation of the results of IL-10 concentrations, in accordance with some demonstrative embodiments.

FIG. 37 is a cross section view of an illustration of a sampling element in accordance with some demonstrative embodiments.

FIG. 38 depicts an exemplary photo of the results of various reactions taking place within a sampling element, according to some demonstrative embodiments.

FIG. 39 is an intensities vs. concentrations graph that can be formulated by the device of the present invention, in accordance with some demonstrative embodiments.

FIG. 40 is a graph depicting OD vs. Concentration for Vitamin B6, in accordance with some demonstrative embodiments.

FIG. 41 is a graph depicting OD vs. Concentration for TNF-alpha, in accordance with some demonstrative embodiments.

FIG. 42 is a graph depicting OD vs. Concentration for IL-10, in accordance with some demonstrative embodiments.

FIG. 43 is an illustration of a device in accordance with some demonstrative embodiments described herein.

FIG. 44, is an illustration of an exemplary chamber for a sampling element, in accordance with some demonstrative embodiments.

### DETAILED DESCRIPTION OF THE INVENTION

According to some demonstrative embodiments, there is provided herein a system for detecting the presence of specific components in breast milk and/or determining and/or measuring the amount and/or concentration thereof.

According to some demonstrative embodiments there is provided herein a system that enables testing and/or analyzing breast milk, for example, for detecting changes in concentrations or amounts of specific biological markers, e.g., immunoglobulins, antibodies and like.

According to some embodiments the system may alert a user of the system when changes occur in concentrations and/or amounts of one or more specific marker(s).

According to these embodiments, for example, the system may enable to detect an elevation in a concentration of a specific immunoglobulin, thereby alerting a possible development of an infection and/or a disease in the breastfeeding mother or a breastfed baby.

A possible example is the detection of antibodies against CMV, which is a virus that can cause significant illness in both nursing mothers and their infants. In case of active infection, the mother's immune system will develop IgM antibodies against the virus. Identification of these antibodies in the mother's milk will serve as an early indication of the mother's condition and may allow preventive measures to be taken to minimize the transmission of the infection to the baby (e.g. temporary halting of breastfeeding, reducing the exposure to maternal fluids, etc.). For example, the system may include one or more devices that can monitor the concentration of one or more markers for a period of time, for example, in specific time intervals, such as on a daily basis, on an hourly basis, on a weekly basis and the like.

According to some demonstrative embodiments, the system may provide an indication of early stages of certain diseases by monitoring and/or identifying changes in specific biomarkers contained in the breast milk, for example, an elevation of a specific antibody in the breast milk of a breastfeeding mom may correlate to the existence of specific pathogens in the breastfed baby.

According to some embodiments the system may provide a detailed summary of concentrations of various markers in the breast milk and enable a care provider to effectively assess the health condition of an infant which is fed by the breast milk.

According to some embodiments, the term “assessment” and “assess” may refer to any suitable indication resulting from the analysis of the breast milk including, for example, a mere summary of concentrations of specific markers, an analysis of the concentrations, a warning regarding the change in one or more specific marker (in amount or concentration), a recommendation of treatment, estimation of a specific disease or medical condition correlating to the amount/concentration or change in one or more markers, and the like.

According to some embodiments, the term “care provider”, “user” and/or “user of the system” may refer to a breastfeeding mother and/or to any individual who provides preventive, curative, promotional or rehabilitative health care services within all branches of health care, including medicine, surgery, dentistry, midwifery, pharmacy, psychology, nursing or allied health professions, and the like.

According to some embodiments, the terms “marker”, “biomarker”, “immunological marker(s)” and/or “biological marker(s)” may refer to any measurable indicator of some biological state or condition. According to some embodiments, the marker may be used to refer to a substance whose detection indicates the presence of a living organism and/or a disease. According to these embodiments, the marker may be measured and/or evaluated to examine normal biological processes, pathogenic processes, or pharmacologic responses to a therapeutic intervention.

According to some embodiments, the term “sampling element(s)” may include, for example, a test strip, a test stick, a dipstick, a vial and/or any suitable piece of material used for biological testing.

According to some embodiments, the sampling element may include one or more components for the analysis of the sampled breastmilk, as explained in detail below. According to some embodiments, the sampling element may further include one or more indicators to indicate the result of the analysis.

According to some embodiments, the sampling element may include one or more magnifying elements, to magnify the results. For example, results may be presented by color upon the indicator of the sampling element, however, since some of the tested elements may be in small amount and/or concentration, e.g., microns, according to these embodiments, the sampling element may include a magnifying glass or window to magnify the resulting color or the presentation of the results.

According to some embodiments, the sampling element may include one or more reagents and/or other suitable molecules that enable a reaction to take place, e.g., a chemical reaction, for example, in the presence of one or more target molecule that may be present in a sampled breastmilk coming in contact with the sampling element.

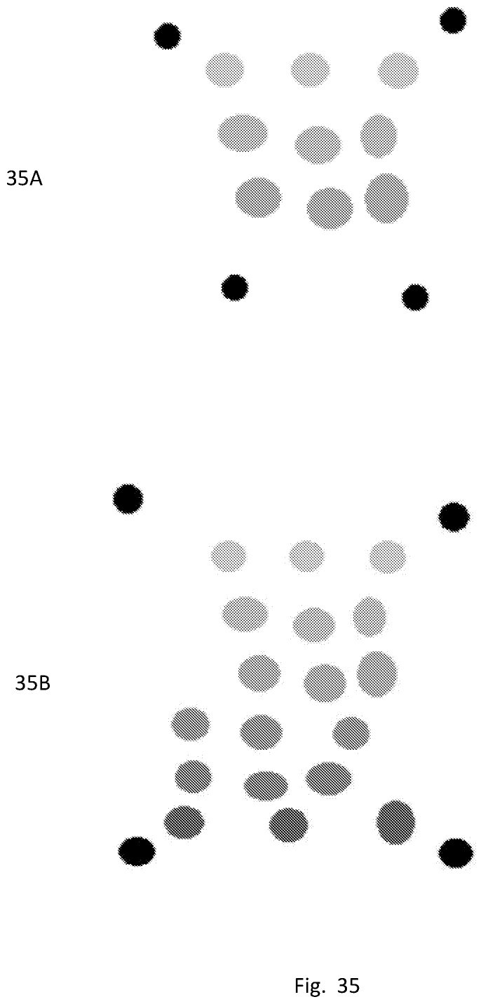

According to some embodiments, the reaction may be visually represented, for example, on one or more portions of the sampling element, as described in detail below and presented in FIGS. 35A and 35B.

According to some embodiments, the term “application(s)” as referred to herein may include, for example, any suitable software that may enable the analysis of data and/or indication of an output based on the analysis. According to some embodiments, the application may be an internal software implemented within the system. According to other embodiments, the application may be installed on an external device, for example, a smart phone and the like. According to some demonstrative embodiments, the sampling element may have one or more indicators that may be read by an application installed upon a smartphone. For example, the sampling element may have an indicator that may be photographed by a smartphone's camera, and analyzed by an application and/or other software to provide an analysis of the results. According to some embodiments the system may provide a recommendation to the care provider regarding actions or measures to be taken with regard through the assessment.

According to some embodiments, the system may enable the care provider of an infant to determine the infant's immunological and/or nutritional needs, e.g., based on the detection and/or measurement described hereinabove.

According to some embodiments, the system may enable a user of the system to determine the immunological and/or nutritional needs of a breastfeeding mother, e.g., based on the detection and/or measurement described hereinabove.

According to some embodiments, the system may include one or more sampling elements adapted to collect a sample of the breast milk and analyze the breastmilk.

Specifically, the sampling elements are element may be adapted to sample maternal milk, for example, by having a specific structure adapted to allow flow of the breastmilk with its unique viscosity, wherein the sampling element may act as a filter, having fibers in various sizes, diameter and or density to specifically allow the flow of breastmilk therethrough. According to some embodiments, the system may include one or more devices adapted to encompass and/or read the one or more sampling elements and analyze the components of the sampled maternal milk.

According to some embodiments, the term “read” as referred to herein with relation to the device of the present invention may include for example, mechanical, computational, optical or any other suitable method for gathering data located on the sampling element, optionally including in situ analysis of the data gathered.

According to some embodiments, the device may enable the detection and/or analysis and/or measurement of specific components of the maternal milk, e.g., as described in detail below. According to some embodiments, based on the detection and/or analysis and/or measurement of specific components of the maternal milk, the device may enable to provide an estimate regarding the nutritional and/or immunological status of an infant consuming the maternal milk.

According to some embodiments, the device of the present invention may enable for an analysis of the photographed results, for example, unlike other methods of analyzing colorimetric reactions.

For example, a typical ELISA is performed on a 96-well plate, and at the end of the assay, when the reaction has occurred and the color developed, the results are acquired using a spectrophotometer or an ELISA reader. The spectrophotometer shoots concentrated beams of light at specific wavelengths at each individual well and records how much of that light passed through the well (or how much has been swallowed). The spectrophotometer then translates this information to a number (with no units) called the Optical Density, or OD, usually ranging from 0 to 2.5. The more concentrated the color of the ELISA reaction, the higher the OD. In order to determine the unknown concentration of a specific target in a sample using ELISA, it is necessary to add to the plate standards of the target at known concentrations. Once the device has given an OD value to each of the wells on the plate, it is possible to use this information together with the known concentrations to plot a linear graph which can be used to predict the unknown concentration.

According to some embodiments, the device of the present invention may analyze the sampling element via photographing the element. According to these embodiments, the sampling element may include one or more reagents which cause one or more reactions with specific components in the analyzed breastmilk.

According to these embodiments, the device may include at least one camera to take a photograph of the sampling element after the reactions have occurred.

According to some embodiments, the camera may include any suitable camera adapted to take photographs, preferably, high resolution photographs. According to some embodiments, the camera may be a full color camera, a single color monochromatic camera, a fluorescence camera or a black and white camera.

According to some preferable examples, the camera may be a monochromatic black and white camera, for example, due to cost considerations.

According to some embodiments, the device may include one or more computers adapted to analyze the pictures of the sampling element using, for example, a predetermined algorithm.

According to some embodiments, as per the algorithm the device may calculate an average of the color as resulting from the one or more reactions in the array. According to some embodiments, the term “Intensity of the color” (Also referred to herein as “intensity of grey”) may relate to a scale of infinite number ranging from 0 to 1, wherein 0 represents white and 1 represents black.

According to some embodiments, the device may assign a number on a scale from 0 to 1, which for example may be used to represent Optical density (“OD”).

According to some embodiments, the device of the present invention may include a camera to take a photograph of the a reaction zone on a sampling element, and at least one computer including a pre-defined algorithm to analyze the photograph. According to some embodiments, the analysis may include applying a transformation equation, e.g., to allocate each pixel within that reaction area of the sampling element a value between 0 to 1 (“the allocated value”).

According to some embodiments, this value is unique to a specific grey color and/or intensity, i.e., each pixel that possesses the exact same tone and/or intensity of color, e.g., grey, will be allocated with the same value.

According to some embodiments, the allocated value may be between 0-1 and without a unit. According to some embodiments, the allocated value may be compared with other data, for example, average standard concentrations of the target molecule, to create a standard graph and a linear regression equation (an injective function). According to some embodiments, from this linear equation it may then be possible to predict the concentration of the tested target molecules.

According to some embodiments, different reactions, for example, enzymatic, colorimetric and/or chemical may reflect the presence (or absence in competitive tests) of a target molecule (e.g. Vitamin B6), e.g., via the color intensity of the reaction.

According to some embodiments, each reaction may be represented by a different color (e.g. 3,3′,5,5′-Tetramethylbenzidine or TMB is a chromogenic substrate used in staining procedures in immunohistochemistry as well as being a visualizing reagent used in enzyme-linked immunosorbent assays and is a white crystal powder that forms a pale blue-green liquid in solution with ethyl acetate.).

According to some embodiments, the sampling element of the present invention may include one or more substances that cause a color change upon contact with one or more target molecules.

According to some preferable embodiments of the present invention, the device of the present invention may analyze the intensity of color for the determination of the presence and/or amount or concentration of a target molecule. According to these embodiments, analyzing the intensity of color, as referred to herein, may relate to the ability to examine the density of color particles per defined area.

According to some embodiments, with regard to imaging and color, the perceived darkness of a spot on the sampling element, as detailed herein, may be caused by the absorption or reflection of light impinging on the sampling element. Differences in density as related to color are also referred to herein as “gray levels”. As the density of a spot in the micro-array increases, the amount of light that is absorbed by the spot increases, while the amount of light reflected from or transmitted by the spot decreases. The absorption of light is inversely proportional to the transmission or reflectance of light, according to the formula:

Density=log₁₀ 1/Transmittance=log₁₀ 1/Reflectance

The term density as used herein may also refer to the degree of blackness or darkness possessed by a spot.

According to some embodiments, with regard to the color intensity of the spots presented on the sampling element, as explained in detail below, the deeper the color, the stronger the presence (or absence) of the tested target molecule. According to some demonstrative embodiments, the device of the present invention may include one or more cameras to photograph the sampling element and at least one computer to analyze the photograph according to a pre-defined algorithm. According to some embodiments, as per the algorithm the computer may equate the color intensity to the concentration of the target molecule. According to some embodiments, the device of the present invention may analyze a black and white photograph (also referred to as a “grey scale photograph”)

According to some embodiments, a monochromatic camera may enable a more effective analysis of the photograph of the sampling element, and may also be cheaper than color cameras, making the entire device of the present invention cheaper.

According to some embodiments, the camera of the device of the present invention may be a Fluorescence camera, wherein the reactions of the sampling element according to these embodiments may be fluorescence reactions.

According to some embodiments, the use of such a scale may enable extrapolation of the analysis results into a linear graph. This scale and subsequent linear graph extrapolated from it may provide an injective function, for example, making it easy to predict the concentrations of target molecules in the analyzed breastmilk sample (wherein each target molecule may have its own function).

According to some embodiments, the calculation results may be compared to various other results, also referred to herein as the “previous results”. The previous results may include the user's results from past analysis, to the average results of other users in the data bank, and to results described in the relevant literature (also referred to herein as “gold standard”).

According to some embodiments, if the results of an analysis indicate a concentration or amount of a target molecule which deviates from the gold standard, the device may indicate various courses of action, such as, consulting a medical professional, consulting with a breastfeeding expert, making dietary changes, referring to a specific supplement, offering a personalized supplement, etc.

According to some embodiments, a pre-determined range of amounts and/or concentrations and/or a pre-determined binary value for each target molecule may be set, for example on the computer of the device, e.g., as part of the algorithm. For example, the pre-determined range may relate to average ranges of target molecules in the breast milk, e.g., Albumin in an average concentration of 14-17 mg/ml. With regard to certain target molecules, e.g., immunological target molecules, the pre-determined binary value might include 1 or 0, or any other suitable parameter to indicate the existence or absence of the target molecule.

According to some embodiments, the pre-determined range and/or value may be set according to literature information or market gold standard.

According to some embodiments, the device of the present invention may read the sampling element and compare, e.g., via the set algorithm, results of an analyzed breast milk sample to the pre-determined range and/or value set therein. According to some embodiments, if the result is below the pre-determined range, the device may provide an indication to the user, for example, with suggestions for possible courses of action (consulting with a nutritionist/breastfeeding consultant, taking supplements, dietary changes etc.). According to some embodiments, if the result is within or above the range, the device may present the result on a scale, for example, with a message indicating the result is on or above the standard (no further action needed).

Immune Factors and Gold Standard:

Some immune factors, such as general immunoglobulins, some cytokines, lactoferrin, etc. are always present in the breastmilk, and only when they pass a certain threshold it is possible to determine that an illness or ailment is developing. Other more specific immune factors, such as CMV-IgM, are only present in the breastmilk when either the infant or the mother has their corresponding disease, therefore according to some embodiments, the predetermined value may be set to show a “present/not present” result. The test for the different cluster of differentiations (e.g. CD-45) is usually performed with a cell count. Exemplary target molecules may include: CD-3 CD-4, CD-8, CD-14, CD19, CD-45 and CD-56 and the like. According to some embodiments, the device may include one or more artificial intelligence components, for example, adapted to constantly added user information, incorporate date from new studies, changing recommendations for a specific user based on the user's feedbacks, etc.

The maternal milk is constantly changing according to the baby's needs, nutritional and immunological alike. It is rich in immunological factors (antibodies) in the first weeks after delivery and it reduced in these factors later on. On the other hand the complement factors are increasing during lactation period. The antibodies' composition is unique to each mother and baby pair, which makes it very dynamic depending on the baby's exact needs at that time. The main component which concentrates on breast milk and is passed through it to the baby is an immunoglobulin called IgA. This antibody serves as the primary defense of the infant against infections, especially in his developing digestive and respiratory systems. IgA's level in breast milk remains high throughout several months postpartum.

According to some embodiments, there is a constant feedback between the baby and the nursing mother influencing the content of the milk. This feedback can influence the composition of the milk both in the terms of nutritional and immunological components. While being fed, the baby's mucus penetrates the breast, exposing its content to the mother's body. In return, the mother's body may react to the needs of the breastfed infant.

According to some demonstrative embodiments, if the breastfed infant is developing a disease, the body of the breastfeeding mother may produce antibodies to be transferred via the breastmilk to protect the infant.

It has been shown that (in mice) mothers that were exposed to allergens can produce antibodies that will protect their newborn becoming allergic. This may have an influence on the development of asthmas and other allergic conditions.

Accordingly, in accordance with some embodiments, in baby's mucus there may be one or more genetic factors that may penetrate the mother's body as well, e.g., via the milk.

According to some demonstrative embodiments, the one or more genetics factors may be utilized for mapping the baby's health condition and/or determining the needs of the baby on top of the immunological components.

Table 1 describes the main immunological components in breast milk, according to different time points within the first year since delivery:

TABLE 1

| Breast milk | Health | | Transitional | Months | Months | Months | Months |
|---|---|---|---|---|---|---|---|
| component | status | Colostrum | milk | 1-3 | 4-6 | 7-9 | 10-12 |
| Total cells | Healthy | 110000- | 113492- | 228395- | 40000- | 97500- | 706667- |
| per ml milk | | 2250000 | 883333 | 255769 | 588542 | 433333 | 1066667 |
| | Infection | — | 183333 | 50000- | 115278- | 37000- | 437 500- |
| | | | | 2867383 | 321918 | 504951 | 1000 000 |
| % | Healthy | 13.2-70.4 | 0.0-1.65 | 0.07-0.45 | 0.0-1.52 | 0.0- | 0.08-0.1 |
| Leukocytes | | | | | | 1.09 | |
| | Infection | — | 18.8 | 0.72-90.5 | 1.1-33.9 | 1.08- | >3 |
| | | | | | | 93.6 | |
| Leukocytes | Healthy | 32175- | 0-3450 | 160-1151 | 0-1025 | 0- | 707-853 |
| per ml milk | | 784080 | | | | 1063 | |
| | Infection | — | 34467 | 2400- | 2164- | 1065- | >30000 |
| | | | | 2594982 | 109130 | 472634 | |
| sIgA (μg | Healthy | 1428-2178 | 131-1096 | 534-1276 | 257-960 | 496- | 401-1044 |
| ml−1) | | | | | | 1350 | |
| | Infection | — | 922 | 36-1418 | 652-1711 | 611- | 714-789 |
| | | | | | | 1509 | |
| IgG (μg | Healthy | 5.3-12.2 | 2.8-9.7 | 6.4-12.4 | 4.6-10.8 | 4.0- | 5.0-16.1 |
| ml−1) | | | | | | 16.4 | |
| | Infection | — | 13 | 6.6-17.1 | 4.8-10.1 | 5.6-14.4 | 7.6-8.8 |
| IgM (μg | Healthy | 16.2-56.1 | 8.2-29.8 | 10.6-14.9 | 6.5-11.6 | 4.2- | 8.8-23.3 |
| m1−1) | | | | | | 23.7 | |
| | Infection | — | 10.2 | 4.5-19.8 | 10.1- | 12.6-21.8 | 14.4-19.3 |
| | | | | | 15.4 | | |
| Lactoferrin | Healthy | 6.3-7.7 | 2.1-5.2 | 2.5-2.9 | 1.9-3.7 | 1.3-4.0 | 1.2-3.9 |
| (g l−1) | | | | | | | |
| | Infection | — | 4.3 | 2.9-3.7 | 2.0-3.7 | 1.6-3.3 | 1.2-3.6 |

Table 2 lists several antigens which can be found in the breast milk of an infected nursing mothers, and to which the infants may be exposed:

TABLE 2

| Antigen in breast milk | Corresponding Illness in infant |
|---|---|
| CMV IgM and CMV IgG | CMV(Cytomegalovirus) |
| Hepatitis B Antigen | Hepatitis B Virus |
| (surface antigen AKA | |
| HBsAg) | |
| Hepatitis C Antigen | Hepatitis C Virus |
| (surface antigen AKA | |
| HBsAg) | |
| HIV1 Antigen (p24) | Human Immunodeficiency Virus Type 1 (HIV1) |
| HIV2 Antigen (p24) | Human Immunodeficiency Virus Type 2 (HIV2) |
| HTLV-I Antigen (gp21 or | Human T-lymphotropic virus Type 1 (HTLV-1) |
| p-24 Core) | |

According to some demonstrative embodiments, the system of the present invention (also referred to herein as a “kit”) may include one or more sampling elements capable of testing different nutritional factors as described below and/or immunological markers that may indicate a development of an infection\illness \disease.

According to some embodiments, the system may further include one or more tubes for the dipping of the sampling element into the breastmilk, and a color calibration board.

According to some embodiments, the system of the present invention is designated for women who breast feed (even partially). According to some embodiments, a nursing mother may test her breast milk at the same time every day, mainly before/after a meal. For example, a mother using the system of the present invention may be asked to pump milk into a bottle or another sealable container and transfer the milk into a designated tube and will dip a sampling element (alternatively, the sampling element may be placed in the tube beforehand) into the tube, making sure the breastmilk is covering the sampling element up to a marked line.

According to some embodiments, the breastmilk sample may also include sample which is not considered to be fresh, for example, milk which has been refrigerated or frozen.

According to some embodiments, after a short incubation period, the sampling element may be placed in a designated slot on a calibration panel of the system, and the mother may be able to use an application to scan the entire panel with her phone camera.

According to some embodiments, the sampling element may be coated with different antibodies, recombinant proteins, synthesized vitamins, hormones, enzymes, etc. for the specific test targets. The tests may for any suitable target molecule which may be present in the breastmilk, including for example, nutritional factors, hormones and/or for immunological components and the like. The reaction may be colorimetric and can be quantitated using a color panel.

According to some demonstrative embodiments, the sampling element may include one or more antibodies, for example, to detect and/or monitor the levels of different nutritional factors such as vitamins, e.g., vitamin A, B1, B6, B12, C, D and the like.

According to some demonstrative embodiments, the sampling element may include one or more antibodies, for example, to detect and/or monitor the levels of different immunological factors such as CD45, sIgA and the like, and/or other antibodies that may act against common antigens that are present as a result of pathogen, e.g., such as Streptococcus pneumoniae.

According to some embodiments, color indication may be the preferred option to demonstrate changes and/or results analyzed by the system of the present invention.

According to some embodiments, the color test may vary, and optionally be dependent on the final substance to be tested.

For example, protein concentration is tested by Bradford, BCA etc.—true, the color change is better visualized by a designated equipment but is quite clear also to the naked eye. pH test can be done with pH strips that changes their color according to the pH.

Number of cells can be estimated according to some enzymatic assay such as CCK.

Total Phosphorus can be measured by different enzymatic assays such as Molybdenum blue method by adding a reducing agent. This can take place as follows:

Orthophosphate reacts with ammonium molybdate to a slightly yellow molybdenum phosphoric acid. By adding a reducing agent, the molybdenum blue is formed, which has a blue color:

PO₄³⁻+12(NH₄)6Mo₇O₂₄+3H⁺→H₃P(Mo₃O₁₀)⁴

H₃P(Mo₃O₁₀)⁴+2SnCl₂+8HCl→H₇P(Mo₃O₁₀)⁴+2H₂(SnCl₆)

pH is a measure of the acidity or alkalinity of a solution. The pH value states the relative quantity of hydrogen ions (H+) contained in a solution. The greater the concentration of H+ the more acidic the solution and the lower the pH. In this relationship, pH is defined as the negative logarithm of hydrogen activity.

The Bradford assay, a colorimetric protein assay, is based on an absorbance shift of the dye Coomassie Brilliant Blue G-250. Under acidic conditions the red form of the dye is converted into its bluer form, binding to the protein being assayed. The dye forms a strong, noncovalent complex with the protein's carboxyl group by Van der Waals force and amino group through electrostatic interactions. During the formation of this complex, the red form of Coomassie dye first donates its free electron to the ionizable groups on the protein, which causes a disruption of the protein's native state, consequently exposing its hydrophobic pockets. These pockets in the protein's tertiary structure bind non-covalently to the non-polar region of the dye via the first bond interaction (van der Waals forces) which position the positive amine groups in proximity with the negative charge of the dye. The bond is further strengthened by the second bond interaction between the two, the ionic interaction. The binding of the protein stabilizes the blue form of the Coomassie dye; thus the amount of the complex present in solution is a measure for the protein concentration, and can be estimated by use of an absorbance reading. The BCA assay primarily relies on two reactions.

First, the peptide bonds in protein reduce Cu²⁺ ions from the copper (II) sulfate to Cu⁺ (a temperature dependent reaction). The amount of Cu²⁺ reduced is proportional to the amount of protein present in the solution. Next, two molecules of bicinchoninic acid chelate with each Cu⁺ ion, forming a purple-colored complex that strongly absorbs light at a wavelength of 562 nm.

The bicinchoninic acid Cu⁺ complex is influenced in protein samples by the presence of cysteine/cystine, tyrosine, and tryptophan side chains.

The amount of protein present in a solution can be quantified by measuring the absorption spectra and comparing with protein solutions of known concentration.

Cell Counting Kit-8 (CCK-8) allows very convenient assays by utilizing Dojindo's highly water-soluble tetrazolium salt. WST-8 [2-(2-methoxy-4-nitrophenyl)-3-(4-nitrophenyl)-5-(2,4-disulfophenyl)-2H-tetrazolium, monosodium salt] produces a water-soluble formazan dye upon reduction in the presence of an electron mediator. CCK-8 allows sensitive colorimetric assays for the determination of the number of viable cells in cell proliferation and cytotoxicity assays. WST-8 is reduced by dehydrogenases in cells to give an orange colored product (formazan), which is soluble in the tissue culture medium. The amount of the formazan dye generated by dehydrogenases in cells is directly proportional to the number of living cells.

According to some embodiments, a phone application may enable scanning of the control panel to compare the colors on the panel (which resemble known concentration of the analyte) to the test color on the sampling element.

According to some embodiments, the application may have an algorithm that may convert the signal into numbers which can indicate the concentration of the analyte, thus enabling the monitoring of the concentrations of various analytes and/or markers over time.

According to some demonstrative embodiments, the application may recognize fluctuations in concentrations/decrease-increase in concentrations/sub-optimal concentration of a marker and/or a nutritional component, thus sending recommendations, for example, for treatment and/or for the supplementation of a component through diet. According to some embodiments, the nursing mother can use the application in order to monitor the nutritional composition of her breastmilk and/or follow possible recommendations provided by the application.

According to some embodiments, the application may enable direct connection to a care provider, for example, the application may send a notification to a physician in case any abnormal results are indicted, e.g., an extreme rise in one or more markers.

According to some embodiments, the application may enable a mother to input data relating to her infant, for example, such as weight and head circumference, in order to improve the complete monitoring of her baby's health and development.

If the mother chooses to combine formula in her infant's diet in addition to breast feeding, the application may recommend which formula (which stage) best fits the infant and complements the mother's milk's composition.

According to some embodiments, the system may include specific sampling elements for different purposes, for example, a nutritional sampling element including, for example, one or more components adapted to identify amounts and/or concentrations of specific nutritional factors in the breast milk; an immunological sampling element including, for example, one or more components adapted to identify amounts and/or concentrations of specific immunological markers in the breast milk; a combined sampling element, including, for example, one or more components adapted to identify amounts and/or concentrations of specific nutritional factors and specific immunological markers in the breast milk.

According to some embodiments, the immunological sampling element may include one or more tests that bind immunological factors such as sIgA, CD-45, IL-10, and/or TNF-alpha. These immunological factors may serve as an alert/red flag for the development of an illness in the mother, the baby or both.

According to some embodiments, the application of the system may recognize an increase in one or more immunological factors (for example, sIgA, CD-45, IL-10, and/or TNF), for example, following the use of a nutritional sampling element, and consequentially may recommend using the immunological sampling element.

According to some embodiments, the system may include a package of sampling elements, e.g., testing sticks, for example, every such package may contain 30 nutritional sticks and at least one immunological strip.

According to some embodiments, the sampling element may contain a plurality of tests, for example, ten to fifteen different tests, for common or dangerous infant diseases, including, for example: Diphtheria: Hand, foot and mouth disease, Infective Diarrhea, Jaundice—Viral Hepatitis, Measles, Meningitis, Molluscum contagiosum, Mumps, Otitis, Pneumonia, Respiratory Syncytial Virus, Scarlatina, Varicella/Chickenpox, Whooping cough, Pertussis and the like.

According to some embodiments, the application of the system may provide different recommendations to the nursing mother using the system of the present invention. For example, the application may recommend monitoring the infant's fever as well as behavioral abnormalities that might indicate the development of a disease. The application may also recommend going to a physician or any other suitable professional such as a nutritionist or breastfeeding consultant and will allow for an immediate online connection to a physician for consulting.

According to some demonstrative embodiments, the device of the present invention may include a light sensor (camera), a Bluetooth component, a battery pack, a USB port and light/color components that signal “on/off”, “Error”, “In Progress” and so on, using generally agreed upon signals (green for “on”, flashing for “in progress” and so on) According to some embodiments of the invention, the system may include at least one device made up of: A designated area for the nutritional/immunological stick, light/color sensors, a Bluetooth component, a battery pack, a memory chip and optionally an LCD screen, a USB port and a temperature sensor.

According to some embodiments, the system may include one or more options or alternatives for the sampling element to be inserted into the—device of the system and consequentially enabling the analysis of the breast milk.

For example, one option of the system would be to use a sampling stick onto which a sample of the breast milk is put, and the stick is inserted into a device of the system for analysis. A second option may include a dipstick, which is dipped into a sample of a breastmilk, wherein the dipstick includes a panel.

According to some demonstrative embodiments, the device may be able to (1) analyze many different target molecules in triplicates according to their exact location (2) standardize/control the coefficient of light, background noise and image quality. (3) Avoid differences resulting in the use of different phones (with different camera components). (4) possess greater sensitivity, which would mean the reaction areas could be comparatively small (this feature may for example save space on the sampling element and allow for more tests on one sampling element. (5) Reduce errors/contamination which might occur due to over handling of the sampling element (due to the fact that the sampling element is inside a chamber, and is inserted into the device directly after milk is introduced).

Alternatively, the device could also include a slot for cuvettes and magnetic sensors. The kit may also include single use cuvettes which come with all the fluids for the reaction already in them. These cuvettes would be used for specific immunological tests. The reactions within the cuvettes may be colorimetric or result in a charged particle which would be drawn to the magnetic sensors.

According to some demonstrative embodiments, the system may also enable the monitoring and/or analysis of the nutritional composition of the breast milk.

With time, the nutritional factors contained within the breast milk dynamically change in concentration and/or amounts.

The composition of the breastmilk (e.g., fats, carbohydrates, proteins and the like) may vary throughout the months of lactation, and also during the day with a maximum 30 minutes after breastfeeding.

When compared to maternal milk, infant formula at different stages include different concentrations of components, as shown in Table 3.

TABLE 3

| | Materna | | | Nutrilon | | | Similac | | | |
|---|---|---|---|---|---|---|---|---|---|---|
| | Phase 1 | Phase 2 | Phase 3 | Phase 1 | Phase 2 | Phase 3 | Phase 1 | Phase 2 | Phase 3 | |
| Energy | 69 | 69 | 66 | 65 | 62 | 63 | 68 | 73.8 | 73.8 | Kcal |
| Protein | 1.46 | 1.58 | 1.66 | 1.3 | 1.4 | 1.5 | 1.4 | 2.8 | 2.8 | gr |
| (Albumin/Casein | | | | | | | | | | |
| 60/40) | | | | | | | | | | |
| Carbohydrates | 7.7 | 7.8 | 8.4 | 7.3 | 8.6 | 8 | 7.4 | 7.5 | 7.5 | gr |
| Dietary | | | | 0.8 | 0.8 | 1.2 | | | | |
| (scGOS/ICFOS) | | | | | | | | | | |
| Lactose | | 7.8 | 8.2 | | | | 7.4 | 7.5 | 7.34 | gr |
| Fat | 3.5 | 3.5 | 2.9 | 3.4 | 3 | 2.8 | 3.71 | 3.6 | 3.62 | gr |
| Saturated Fat | 1.56 | 1.5 | 1.2 | 1.4 | 1.2 | 0.7 | 1.2 | 1.28 | 1.28 | gr |
| Trans Fat | 0.5> | 0.5> | 0.5> | | | | 0.5> | 0.5> | 0.5> | gr |
| Cholesterol | 4 | 4 | 1.9 | | | | 0.7 | 1.7 | 1.7 | Mg |
| Linoleic Acid | 0.478 | 0.63 | 0.531 | 0.45 | 0.4 | 0.383 | 0.64 | 0.68 | 0.68 | gr |
| (LA) | | | | | | | | | | |
| Alpha-Linolenic | 0.05 | 0.066 | 0.049 | 0.08 | 0.07 | 0.68 | 0.05 | 0.06 | 0.06 | gr |
| (ALA) | | | | | | | | | | |
| Arachidonic | 13 | | | 11 | 8.6 | 1 | | | | Mg |
| (ARA) | | | | | | | | | | |
| Docosahexaenoic | 13 | | | 10 | 8.6 | 12 | | | | Mg |
| Acid (DHA) | | | | | | | | | | |
| Sodium | 23 | 27 | 34.5 | 17 | 23 | 26 | 18 | 36.4 | 36.4 | Mg |
| Vitamin A | 82 | 86 | 87 | 54 | 66 | 65 | 57 | 79 | 79 | Mcg |
| | | | | | | | | | | RE |
| Vitamin D | 1.33 | 1.2 | 1.2 | 1.2 | 1.4 | 1.7 | 0.86 | 1.25 | 1.25 | Mcg |
| Vitamin E | 1.3 | 1.78 | 1.56 | 1.1 | 1.2 | 1.1 | 1.9 | 1.3 | 1.3 | Mg- |
| | | | | | | | | | | TE |
| Vitamin K | 6 | 8.4 | 7.9 | 4.4 | 5 | 4.9 | 6.7 | 6.1 | 6.1 | Mcg |
| Vitamin B1 | 0.1 | 0.11 | 0.1 | 51 | 54 | 50 | 0.08 | 0.097 | 0.097 | Mg |
| Vitamin B2 | 0.12 | 0.12 | 0.15 | 121 | 120 | 124 | 0.14 | 0.12 | 0.12 | Mg |
| Niacin (B3) | 0.58 | 0.73 | 0.73 | 0.43 | 0.43 | 0.44 | 0.71 | 1.25 | 1.25 | Mg |
| | | | | | | | | | | NE |
| Vitamin B6 | 0.08 | 0.08 | 0.09 | 38 | 93 | 40 | 0.04 | 0.14 | 0.14 | Mg |
| Vitamin B12 | 0.23 | 0.27 | 0.3 | 0.21 | 0.18 | 0.14 | 0.19 | 0.291 | 0.29 | Mcg |
| Folic Acid | 13 | 18 | 14 | 13 | 16 | 13 | 9.5 | 13.1 | 13.1 | Mcg |
| Vitamin C | 12 | 13 | 13 | 9.2 | 9.9 | 15 | 10 | 10.9 | 10.9 | Mg |
| Biotin | 2.6 | 2.9 | 3.3 | 1.4 | 1.4 | 1.6 | 2.5 | 3.3 | 3.3 | Mcg |
| Pantothenic acid | 0.4 | 0.5 | 0.6 | 343 | 344 | 339 | 0.4 | 0.43 | 0.43 | Mg |
| (B5) | | | | | | | | | | |
| Calcium | 58 | 81 | 79 | 57 | 65 | 91 | 53 | 115 | 115 | Mg |
| Phosphorus | 37 | 48 | 56.6 | 32 | 36 | 50 | 28 | 66.2 | 66.2 | Mg |
| Magnesium | 6.6 | 8.5 | 6.9 | 5.1 | 4.8 | 5.6 | 5.1 | 8.7 | 8.7 | Mg |
| Iodine | 9.5 | 14 | 14 | 12 | 13 | 13 | 13 | 22.4 | 22.4 | Mcg |
| Iron | 0.82 | 1.23 | 1.24 | 0.53 | 1 | 1.2 | 1.2 | 1.2 | 1.2 | Mg |
| Zinc | 0.61 | 0.55 | 0.73 | 0.52 | 0.51 | 0.89 | 0.5 | 0.55 | 0.55 | Mg |
| Selenium | 2.8 | 4.1 | 4.1 | 1.7 | 1.7 | 1.5 | 1.1 | 1.1 | | Mcg |
| Potassium | 71 | 67 | 90 | 68 | 74 | 70 | 83 | 128 | 128 | Mg |
| Copper | 49 | 48 | 44 | 40 | 43 | 41 | 51 | 66 | 0.066 | Mcg |
| Manganese | 6.6 | 7.5 | 5.8 | 7.7 | 7 | 7.6 | 13 | 9.5 | 9.5 | Mcg |
| Chloride | 63 | 44 | 52.4 | 42 | 47 | 41 | 44 | 84.3 | 84.3 | Mg |
| Choline | 16 | 10.4 | 9 | 12 | 10 | 10 | 10 | 17.64 | 17.64 | Mg |
| Taurine | 6 | 6.9 | 4.6 | 5.3 | 5.2 | 5.4 | 4.5 | 5 | 5 | Mg |
| Inositol | 4 | 8.2 | 7.7 | 3.9 | 3.6 | | 4 | 3.3 | | Mg |
| Carnitine | 0.9 | | | 1.6 | 0.95 | | 1.1 | | | Mg |
| Nucleotides | 3.1 | 3 | 3 | 3.2 | 3.2 | | 7.2 | | 7.2 | Mg |
| Humidity | | | 2.5 | | | | | | | % |

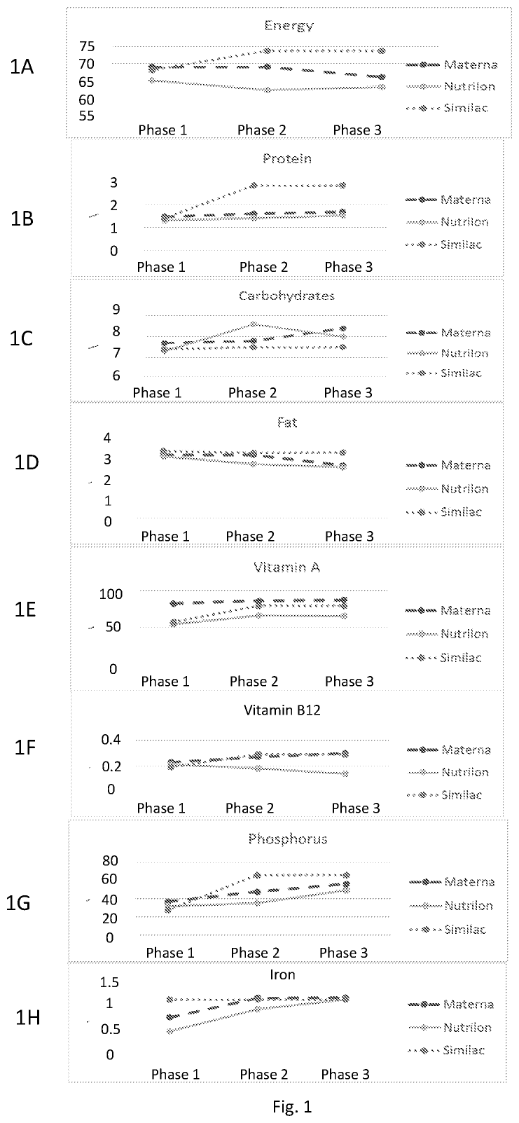

Reference is now made to FIG. 1, which illustrates graphs 1A-1H depicting the changes in different components in various infant formulas (Materna®, Similac® and Nutrilon®) during stages 1, 2 and 3 of the formulas, in accordance with some demonstrative embodiments. According to some embodiments, graph 1A depicts the changes in Energy levels (Kcal) in the various phases 1, 2 and 3 of exemplary leading infant formula brands—Materna®, Similac® and Nutrilon®.

According to some embodiments, graph 1B depicts the changes in Protein concentrations (grams/100 ml) in the various phases 1, 2 and 3 of exemplary leading infant formula brands—Materna®, Similac® and Nutrilon®.

According to some embodiments, graph 1C depicts the changes in Carbohydrate concentrations in the various phases 1, 2 and 3 of exemplary leading infant formula brands—Materna®, Similac® and Nutrilon®.

According to some embodiments, graph 1D depicts the changes in Fat concentrations in the various phases 1, 2 and 3 of exemplary leading infant formula brands—Materna®, Similac® and Nutrilon®.

According to some embodiments, graph 1E depicts the changes in Vitamin A concentrations in the various phases 1, 2 and 3 of exemplary leading infant formula brands—Materna®, Similac® and Nutrilon®.

According to some embodiments, graph 1F depicts the changes in Vitamin B12 concentrations in the various phases 1, 2 and 3 of exemplary leading infant formula brands—Materna®, Similac® and Nutrilon®.

According to some embodiments, graph 1G depicts the changes in Phosphorus concentrations in the various phases 1, 2 and 3 of exemplary leading infant formula brands—Materna®, Similac® and Nutrilon®.

According to some embodiments, graph 1H depicts the changes in Iron concentrations in the various phases 1, 2 and 3 of exemplary leading infant formula brands—Materna®, Similac® and Nutrilon®.

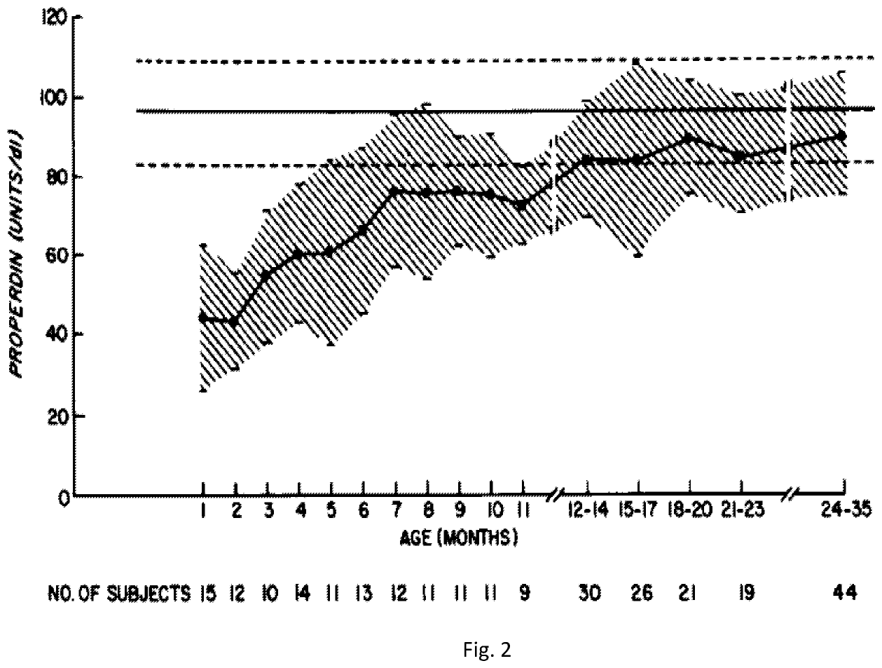

Reference is now made to FIG. 2 which illustrates a graph depicting the change in properdin in correlation to the age of the infant, in accordance with some demonstrative embodiments. Complement factors, such as: Clq, C2, C3, C4, C5, C6, Factor B, Properdin, C3b inactivator (C3Bina) and β1H are increasing during lactation when Properdin levels in the infant serum are changing significantly during the first 3 years.

As shown in FIG. 2, Properdin levels in infant's ages 1-35 months. The solid dark line represents the mean and the shaded areas±1 SD. The mean adult properdin level and ±1SD are given by the horizontal solid and dotted lines, respectively. The number of subjects in each age group is listed at the bottom of FIG. 2.

The following are embodiments of possible structures and modes of operation of the sampling element:

According to some demonstrative embodiments, the mechanism of the sampling element may be based on Lateral flow tests as are frequently applied in quick pregnancy tests on absorbing fibers/pad/membrane. For example, based on antibodies and an enzymatic reaction for color. According to some embodiments, the sampling element may operate based on chemical reactions, enzymatic reactions etc. with or without antibodies.

According to some embodiments, the sampling element may have an edge that may be placed in a sample liquid, e.g., breastmilk. According to some embodiments, the sampling element may include fibers that absorb the liquids forcing it to move from higher concentration (where the sample is originally placed) to the lower concentration. This directional flow can be achieved by capillaries as well.

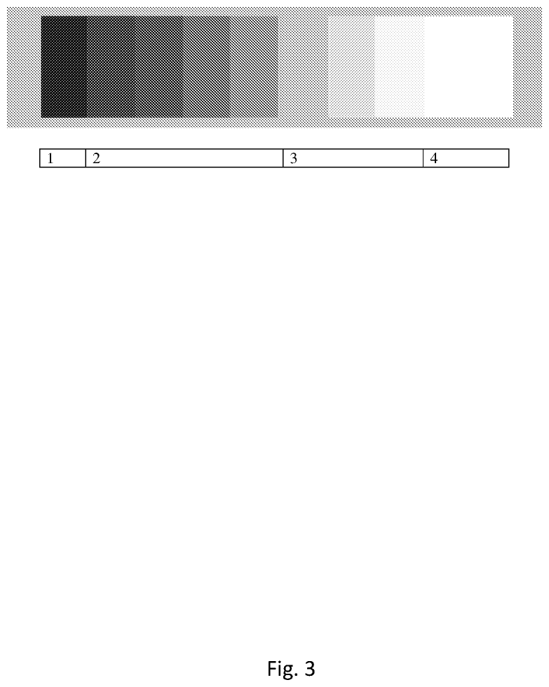

Reference is now made to FIG. 3. Which illustrates an exemplary scheme for assisting in the identification of a suitable nutritional formula for an infant, in accordance with some demonstrative embodiments as shown in FIG. 3, the strength of the color may indicate the stages of the formula that the baby need.

According to some embodiments, the formation of the color in the different enzymatic reactions is dependent on the amount of the tested material. For protein concentration the Coomassie dye forms a strong, noncovalent complex with the protein's carboxyl group by Van der Waals force and amino group through electrostatic interactions. According to some embodiments, the higher the protein concentration is, the stronger the color indication is.

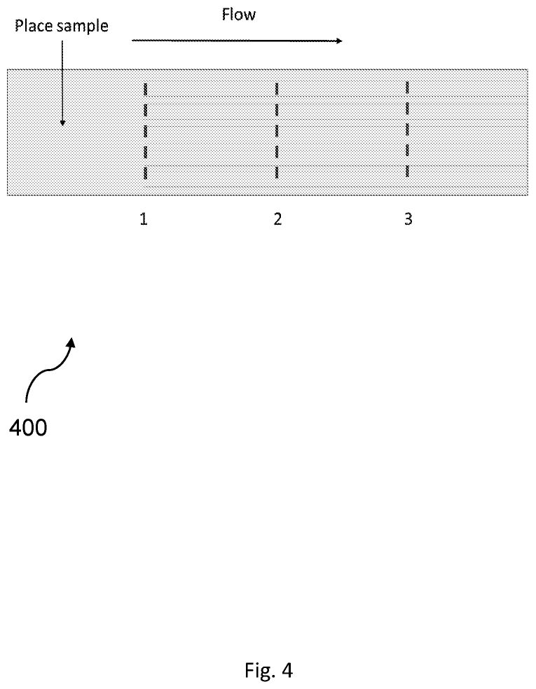

According to some demonstrative embodiments of the present invention the system may include the identification of a suitable nutritional formula for an infant via at least one of the following parameters: number indicators, color indicators, marking indicators, Latin letter indicators, and/or any other suitable electronic indicators, e.g., sound, beeping and the like. Reference is now made to FIG. 4 which is an illustration of a sampling element (SE) 400, and the movement therein of a sample liquid, in accordance with some demonstrative embodiments.

As shown in FIG. 4, SE 400 may include a sampling area, to collect the sample and at least three separate zones, zone 1, zone 2 and zone 3, through which a sampled liquid may pass.

According to some embodiments, zone 1 may include antibodies for the tested targets (e.g. vitamin A, CD45 etc.). These antibodies are conjugated to an enzyme (or other component—e.g. HRP) that can optionally induce a color change.

According to some embodiments, when an analyte flows through its corresponding antibody (zone 1) the antibody may bind to the analyte and carry on to zone 2. At this station, there are corresponding antibodies (these may include the same protein but different epitope) that may be bound to the layer.

Zone 2 may also include particles that may induce the color release from the conjugated enzyme (e.g. Cumaric+Luminol+H₂O₂). When a specific analyte is present, the antibodies are binding to the analyte and the color change will occur at zone 2. When the analyte is not found in the tested example, or—is present in a low concentration, the color may be formed in zone 3. The intensity of the color is dependent on the amount of analytes in the sample which can be quantify by the device.

According to some demonstrative embodiments, Zone 3, is at the end of the flow, therefore the antibodies from zone 1 that are conjugated to an enzyme (e.g. HRP) and did not bind to any antigens along zone 1 or zone 2 will arrive to zone 3, e.g., by flow.

According to some embodiments, zone 3 may also include particles that induce the color release from the conjugated enzyme (e.g. Cumaric+Luminol+H₂O₂).

According to some embodiments, SE 400 may include additional zones having the same or other functionalities as zones 1, 2 and/or 3.

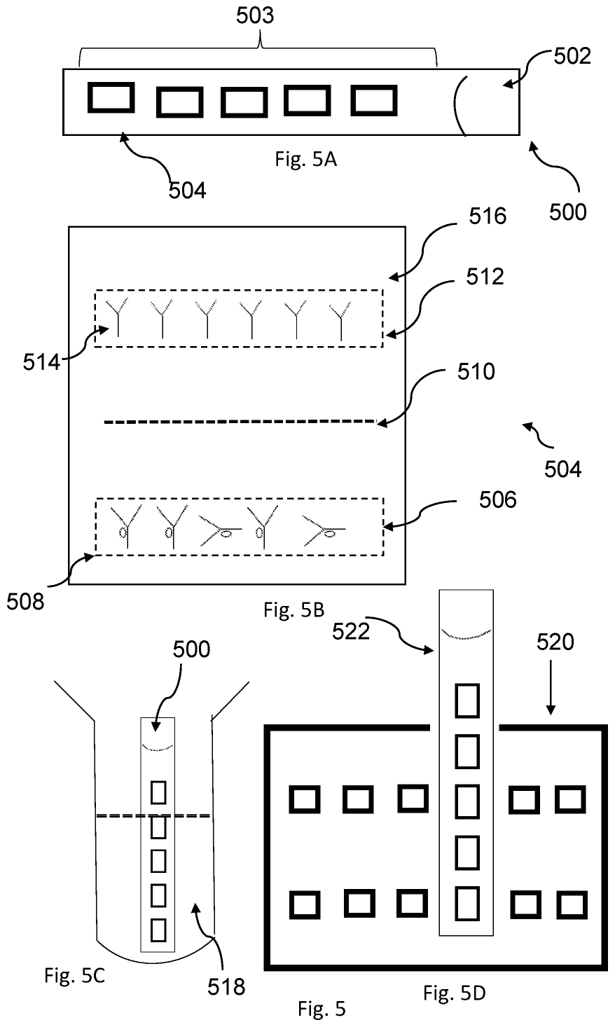

According to some embodiments, the sampling element of the present invention may include a dipstick, as explained in detail below with regard to FIG. 5.

According to some embodiments, in contrast to a strip sampling element having a lateral flow, a dipstick sampling element may be built with layers. At the first layers (outer station), there may be antibodies in a dehydrated form and they may be conjugated to an enzyme or a chromophore (such as colored polystyrene particle) or to colloidal metals. According to some embodiments, this may also be referred to as the “reaction zone” wherein the antibodies are conjugated to the liberation pad in a leachable (easily detachable) manner.

According to some embodiments, when an antigen/analyte binds to the antibody, the complex (e.g. antibody-antigens) moves forward to the second layer, also referred to herein as “the test zone” (deeper layer). At this station there are corresponding antibodies (same protein but different epitope) that may be bound to the layer. When the complex reaches the second station, it may bond to the antibody that is bound to place, thus stopping the flow of the complex. At the second station, the enzyme, the chromophore or the colloidal metal that was bound to the antibodies/enzymes is either released or otherwise causes a reaction which results in a color change. The intensity of the signal (color) is in relation to the concentration of antigen/analyte in the sample.

For an alternative in which the strip is inserted into the device before introducing it to the breast milk, the same principles would apply, but the lateral flow would usually be vertical and not horizontal due to the fact that there is a designated place for the milk (e.g., since entire stick is not wetted).

The antibodies based assay is an indirect (“sandwich”) enzyme-linked immunosorbent assay (ELISA) when there are two antibodies that are corresponding for the same protein targeting different epitops. Alternatively, the first antibody is a monoclonal (highly specific) and the second antibody is a polyclonal antibody.

Reference is now made to FIGS. 5A-5D which illustrate a dipstick sampling element 500 with a panel, according to some demonstrative embodiments.

As shown in FIG. 5A, sampling element 500 may include a holding area 502 and a dipstick body 503. According to some embodiments, a user of element 500 may hold element 500 by holding area 502 an immerse body 503 into a sample of breastmilk to be tested.

According to some embodiments, body 503 may include one or more testing area 504.

FIG. 5B further describes testing area 504 in more detail: according to some embodiments, area 504 includes a liberation pad 506 (also referred to herein as “reaction zone 506”).

According to some embodiments, reaction zone 506 includes a plurality of antibodies 508, wherein antibodies 508 may be conjugated to enzymes or chromophores.

According to some embodiments, area 504 may include a porous membrane 510.

According to some embodiments, membrane 510 may have at least two functions, wherein a first function of membrane 510 may include filtering macromolecules that are in the sample and may reduce the performance of the antibodies, and wherein the second function of membrane 510 may include stalling of the bound antigens (bound to the antibodies), e.g. for improving the chances of a stronger adherence.

According to some embodiments, area 504 includes a second layer 512 (also referred to herein as “test zone 512”), which contains bound antibodies 514 for specific components in the analyte being tested.

According to some embodiments, area 504 includes a hydrophobic center 516 comprising the dipstick. According to some embodiments, in contrast to the lateral flow stick, wherein the sample is placed in a designated area, which in turn determines the direction of the flow, the dipstick may be exposed and circumvent by the milk. Therefore, a partition (hydrophobic) blocks the milk from one end forcing it to be absorbed in a directional manner. Every square on the dipstick may be an individual unit, separated by the hydrophobic partition.

Reference is made to FIG. 5C which illustrates sampling element 500 immersed in a tested sample (breast milk 518).

Reference is made to FIG. 5D which depicts a used sampling element 522 inserted into a panel 520.

According to some embodiments, after a sampling element 522 has been brought into contact with a sampled breastmilk, a chemical reaction has occurred, and is reflected upon sampling element 522 in terms of a color change.

According to some embodiments, after such a reaction has occurred, sampling element 522 may be inserted into panel 520 which has a variety of detection colors for reference.

According to some embodiments, the colors of element 522 may be compared to the reference colors of panel 520 to determine the existence and/or estimated concentration of a specific analyte or a group of analytes in the tested breastmilk.

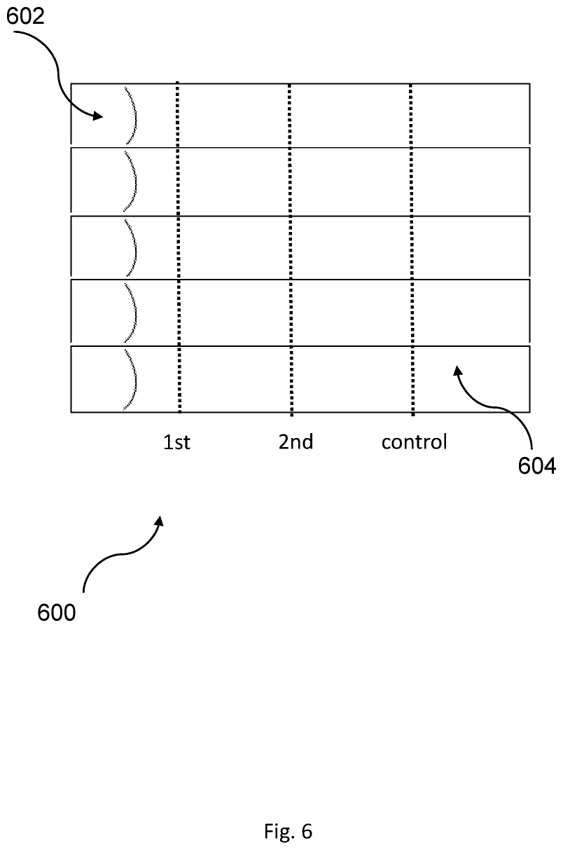

Reference is now made to FIG. 6, which depicts a horizontal stick flow sampling element 600, according to some demonstrative embodiments.

Horizontal element 600 may comprise an edge for placing a breastmilk sample 602, and a body which may contain absorbing surfaces as previously described, for example, the absorbing surfaces may include any suitable fibers, pads and/or membranes.

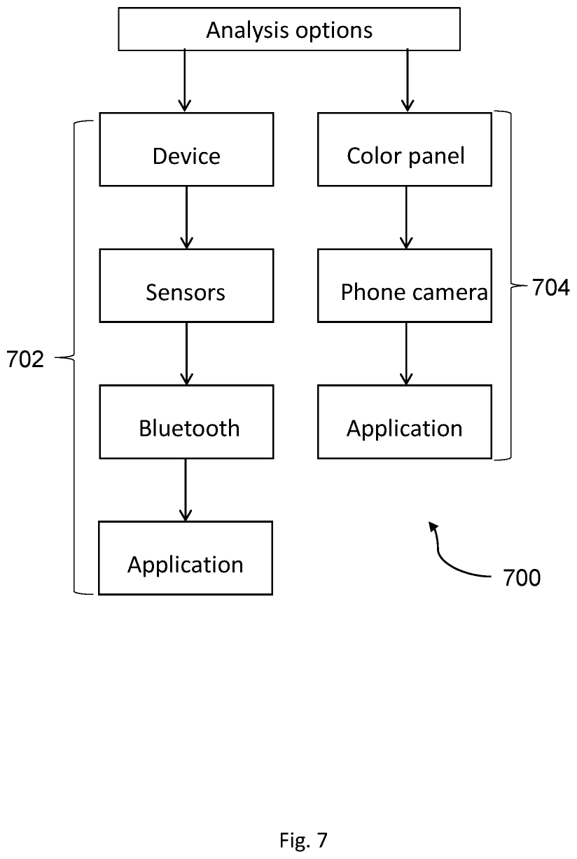

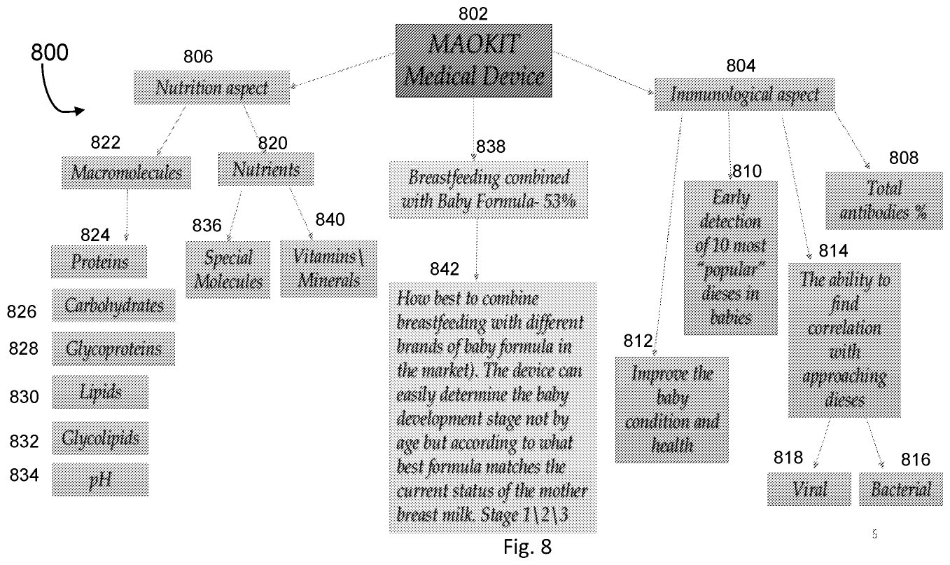

According to some embodiments, the absorbing surfaces may be arranged in 5 lanes, separated by a hydrophobic strip (604). Reference is now made to FIG. 7, which depicts a flow chart of different possible analysis options 700 for sampling and analyzing breast milk using the system of the present invention, according to some demonstrative embodiments.

According to some demonstrative embodiments, the system of the present invention may operate in 3 modes of operation:

1. Immunological—allows the parents immunological monitoring of the baby. Significant tool in predicting a developing/disease in the baby before it shows clinical symptoms whether it is viral/bacterial or a specific illness that an early detection of it can really improve the baby condition and health (like: meningitis, asthma, Pneumonia etc.) or even enabling the parent to make an appointment with the physician (instead of going to work).

2. Nutritional—allowing any mothers, anywhere in the world, monitoring and improving their specific composition of breastmilk in terms of significant macromolecules and vitamins and nutrients. The mother will be able to save the data of each test on here phone and see how simple changes in her nutrition can impact her milk over time.

3. Breastfeeding combined with Baby Formula—How best to combine breastfeeding with supplementary nutrition (AKA infant food supplements, 53% of the market). All baby food supplements look the same on the supermarket shelves. The choice of product to purchase relies mostly on recommendation.

According to some embodiments, the system of the present invention enables to determine the baby development stage not by age but according to what best matches the current status of the mother breast milk.

According to some embodiments, analysis options 700 may include option 702 including transfer of data from the device, to sensors, e.g., a camera, via Bluetooth and yield a result in the application.

According to some embodiments, option 702 route is applicable whether the sampling element is a horizontal stick or a dipstick or microarray stick.

According to some embodiments, once the breastmilk sample is placed on the stick and/or the stick is dipped in the sample (breastmilk testing actions differ depending on the stick's mechanism), the stick may then be inserted into its allotted slot in the device. According to some embodiments, coating the inner surface of this slot are light/color sensors that can analyze the results of the colorimetric tests on the stick and/or results of a fluorescence reaction. The device may report these findings via Bluetooth to a smartphone application. The application may translate the results into easy-to-understand data, and provide the user with conclusions of the analysis and/or recommendations.

According to some embodiments, analysis options 700 may include option 704 may be applicable for both the horizontal stick and the dipstick. According to some embodiments, once the breastmilk sample is placed on the stick/the stick is dipped in the sample (breastmilk testing actions differ depending on the stick's mechanism), the stick is placed in the middle of a color control panel.

According to some embodiments, a user of the device of the present invention may use a smartphone camera to scan the panel with the stick. An application installed upon the smartphone, may then compare the colored results on the stick to the panel and provide an easy to understand numeric result for each test. The application may then provide the user with possible recommendations, for example, recommended changes in diet or habits if necessary.

Reference is now made to FIG. 8 which illustrates possible uses for the system 800 of the present invention (also referred to herein as “MAOKIT Medical Device”).

According to some embodiments, as shown in FIG. 8, system 800 may include a device 802 (also referred to herein as MAOKIT medical device 802), which is adapted to analyze the nutritional aspect 806 of breastmilk, adapted to provide recommendations with regard to the desired nutritional aspects of an infant receiving combined breastfeeding and formula nutrition 838 and is also adapted to analyze the immunological composition of the breastmilk 804, and optionally provide alerts with relation thereto.

According to some embodiments, device 802 may be adapted to analyze the composition of breastmilk providing information on the nutritional aspect 806 of the milk.

According to some embodiments, device 802 may include one or more elements that may enable the sampling of the milk and the analysis of the composition thereof, for example, via a chemical reaction.

According to some embodiments, nutritional aspect 806 may include the analysis of macromolecules 822 and/or nutrients 820.

According to some embodiments, the analysis of macromolecules 822 may include the analysis of protein 824, carbohydrates 826, glycoproteins 828, lipids 830, glycolipids 832 and pH level 834.

According to some embodiments, the preferred molecules to be tested include, for example: sIgA, IgG, IgM, CD-45 (Leukocytes), Lactoferrin, IL-10, TNF-α, INFγ, HMOs, Macrophages, Lymphocytes, IL-6, TGF-β1, pIgR, Propedin and α-Lactalbumin.

According to some embodiments, device 802 may analyze and/or determine the amount and/or concentration of each of the macromolecules 822 and provide an output detailing the amount and/or concentration of each of macromolecules 822 in the examined breast milk.

According to some embodiments, routine examination of the breast milk using device 802, for example, on a daily basis, may enable monitoring of macromolecules 822. According to some embodiments, device 802 may alert a user of device 802 when an abnormal elevation and or decrease occurs in one or more of macromolecules 822.

According to some embodiments, the analysis of nutrients 820 may include the analysis of special molecules 836 which include, for example Omega 3, Omega 6, Folic acid, Biotin, Choline, Niacin, and/or vitamins/minerals 840, including for example, Vitamin A, Thiamin (B1), Riboflavin (B2), Pyridoxine (B6), B12, Vitamin C, Vitamin D, Vitamin E, and Vitamin K.

According to some embodiments, device 802 may analyze and/or determine the amount and/or concentration of each of the nutrients 820 and provide an output detailing the amount and/or concentration of each of nutrients 820 in the examined breast milk.

According to some embodiments, routine examination of the breast milk using device 802, for example, on a daily basis, may enable monitoring of nutrients 820. According to some embodiments, device 802 may alert a user of device 802 when an abnormal elevation and or decrease occurs in one or more of nutrients 820.

According to some embodiments, device 802 may be adapted to provide recommendations with regard to the desired nutritional aspects of an infant receiving combined breastfeeding and formula nutrition 838.

According to some demonstrative embodiments, and as described in detail hereinabove, many mothers choose to supplement breastfeeding with a baby formula in order to provide for the complete nutritional needs of their child.

According to some embodiments, at present, an infant is fed with a suitable formula according to the age of the infant. For example, a 3 month old baby will be receiving a stage 1 formula. However, the composition of each baby formula does not necessarily correspond to the actual nutritional needs of the infant.

According to some embodiments, device 802 may analyze the breast milk of a mother providing combined nutrition to the infant (both breastfeeding and a baby formula), and determine the actual breastmilk composition.

According to some embodiments, based on the actual concentration and/or amount of components found in the analyzed breastmilk device 802 may provide a recommendation 842 on how to combine breastfeeding with different brands and/or stages of baby formula.

According to some embodiments, based on the actual concentration and/or amount of components (target molecules) found in the analyzed breastmilk device 802 may provide a recommendation 842 on how to modify the breastfeeding mother's diet.

According to some embodiments, device 802 may determine the development stage of the breastfed baby not according to the age of the baby, but according to what formula best matches the current status and/or composition of the mother's breastmilk.

According to some embodiments, device 802 may provide a recommendation of the specific brand and/or stage of the baby formula which best suits the actual needs of the baby, for example, device 802 may analyze the breastmilk of a mother breastfeeding a 5 month old baby and determine that the baby actually needs a stage 2 formula and not a stage 1 formula, based on the actual composition of the breast milk analyzed.

According to some demonstrative embodiments, device 802 may analyze the immunological aspect 804 of a breastmilk.

According to some embodiments, device 802 may determine the actual percentage of different antibodies in breastmilk 808, for example, as detailed in Table 1 above.

According to some embodiments, device 802, when used on a regular basis, for example, on a daily basis, may enable the ongoing monitoring of the concentration and/or amount of different antibodies in the analyzed breastmilk.

According to some embodiments, device 802 may alert when the level of one or more antibodies may rise or decrease, for example, above or below a certain predefined level, e.g., an abnormal level.

Table 4 below details average breastmilk concentration of certain elements when the breastfed baby is healthy and when the breastfed baby is sick.

TABLE 4

| | Average Breastmilk | Average Breastmilk |
|---|---|---|
| | Concentration | Concentration when Sick |
| Molecule | when Healthy baby | baby |
| CD45 | 2,122 cells/ml BM | 5,655 cells/ml BM |
| macrophage | 300 cells/ml BM | 1,220 cells/ml BM |
| TNFα | 2.91 ± 1.51 pg/ml BM | 3.66 ± 1.68 BM |
| Total | 325 cells/ml BM | 2,474 cells/ml BM |
| lymphocytes | | |
| Neutrophils | 813 cells/ml BM | 2,941 cells/ml BM |

Source: Riskin, Arieh, et al. “Changes in immunomodulatory constituents of human milk in response to active infection in the nursing infant.” Pediatric research 71.2 (2011): 220-225. According to some embodiments, device 802 may enable, based on the analysis of immunological aspect 804 of the breastmilk, to detect the early stages of common diseases 810, in the breastfeeding mother and/or breastfed infant, for example, device 802 may enable the early detection of the most common 10 diseases often found in babies.

According to some embodiments, often, an increase in a specific antibody in the breast milk may correspond with the existence of specific pathogens, existing in the baby and/or mother.

According to some embodiments, device 802 may enable to detect a correlation between specific antibody levels in the breast milk and specific upcoming diseases related to specific viral pathogens 818 and/or specific bacterial pathogens 816, for example, as set forth in Table 2 and Table 5 below, which lists common exemplary diseases and their relative pathogens.

TABLE 5

| | Disease | Pathogen (example) |
|---|---|---|
| 1 | Diphtheria | bacterium Corynebacterium diphtheriae |
| 2 | Hand foot and mouth disease | Coxsackievirus A16 & Enterovirus 71 |
| | | (EV-71) |
| 3 | Infective Diarrhea | rotavirus |
| 4 | Jaundice-Viral Hepatitis | Hepatitis A, B, C, D, E |
| 5 | Measles | Measles virus |
| 6 | Meningitis | Neisseria meningitides & Streptococcus |
| | | pneumoniae |
| 7 | Molluscum contagiosum | Poxvirus (MCV) |
| 8 | Mumps | Mumps virus |
| 9 | Pneumonia | Streptococcus pneumoniae |
| 10 | Respiratory Syncytial Virus | Syncytial Virus |
| 11 | Scarlatina | group A streptococcus |
| 12 | Varicella/Chickenpox | varicella zoster virus (VZV) |
| 13 | Whooping cough/Pertussis. | Bordetella pertussis. |

According to some embodiments, the analysis of the immunological aspect 804, as performed by device 802 may contribute and/or improve the condition and/or health of a baby 812.

According to some embodiments, device 802 may assist in the improvement of the overall condition and/or health of a baby due to early diagnosis of possible deficiencies in the breast milk content (e.g., low levels of HMOs which might lead to discomfort or digestive tract infections, low levels of Omega 3 or Omega 6 which might lead to slower or deficient brain development) and/or early diagnosis of possible illnesses, both of which lead to early treatment.

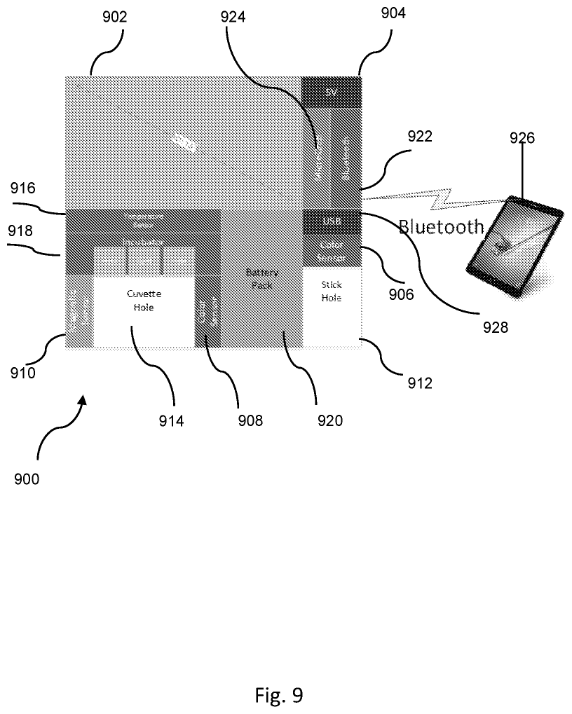

According to some embodiments, device 802 may also uncover “hidden” diseases, such as meningitis, which may often be misdiagnosed as the common cold or a light infection at its early stages. For example, device 802 may assists in the early detection of these diseases by specifically testing for meningitis antigens/antibodies, e.g., using an immunological sampling element. Reference is now made to FIG. 9 which illustrates a system 900 and the components thereof, in accordance with some demonstrative embodiments.

As shown in FIG. 9, system 900 may include screen 902. According to some embodiments, screen 902 may be an LCD screen adapted to present the results analyzed by system 900.

According to some embodiments, screen 902 may include any suitable output device for presentation of information in visual or tactile form, including for example, Cathode ray tube display (CRT), Light-emitting diode display (LED), Electroluminescent display (ELD), Electronic paper, E Ink, Plasma display panel (PDP), Liquid crystal display (LCD), High-Performance Addressing display (EPA), Thin-film transistor display (TFT), Organic light-emitting diode display (OLED), Surface-conduction electron-emitter display (SED) (experimental), Field emission display (FED), Laser TV, Carbon nanotubes, Quantum dot display, Interferometric modulator display (IMOD), Digital micro shutter display (DMS).

Screen 902 may also include three-dimensional displays, including for example, Swept-volume display, Varifocal mirror display, Emissive volume display, Laser display, Holographic display, Light field displays and the like.

According to some preferred embodiments, screen 902 is an LCD screen.

System 900 may also include power supply source 904. According to some embodiments, source 904 may include any power supply coming from the electric power grid, such as an electrical outlet, energy storage devices such as batteries or fuel cells, generators or alternators, solar power converters, or another power supply.

According to some preferred embodiments, source 904 is a 5 volt battery. System 900 may also have a built-in or separate battery pack storage unit 920.

System 900 may also include one or more sensors that can translate the results from the nutritional/immunological tests to easy-to-understand numerical values. According to some embodiments, these can be color/light/fluorescence sensors 906 and 908, and/or magnetic sensor 910.

System 900 may include one or more inputs to which the sample can be inserted, either as a stick hole 912 or a cuvette hole 914, depending on the method of use chosen by the user.

System 900 may also include a Bluetooth component 922 that communicates with a dedicated application 926. This application may be accessed via a mobile phone, laptop, or any other electronic device that supports Bluetooth and onto which the application can be installed.

According to some preferred embodiments, application 926 is accessed via mobile phone.

According to some embodiments, system 900 may include an internal memory device 924 (also referred to herein as “microchip 924”) that can also be expanded and used outside the 900 system. The memory device may allow continuous data storage, its retrieval and display on the device screen or on external devices.

According to some embodiments, system 900 may also have a USB port 928 which enables additional data transfer option from and to system 900.

According to some embodiments, system 900 may include an incubator 918 having one or more incubation lights, to enable the incubation of the analysed maternal milk. According to some embodiments, incubator 918 may include any suitable device and/or array of device or components to enable optimal temperature, humidity and/or other conditions such as the carbon dioxide (CO₂) and oxygen content to allow the growing and/or development of microbiological cultures. According to some embodiments, incubator 918 may include an adjustable heater, typically going up to 60 to 65° C., most preferably, most preferably approximately 37° C., e.g., to provide optimal growing conditions.

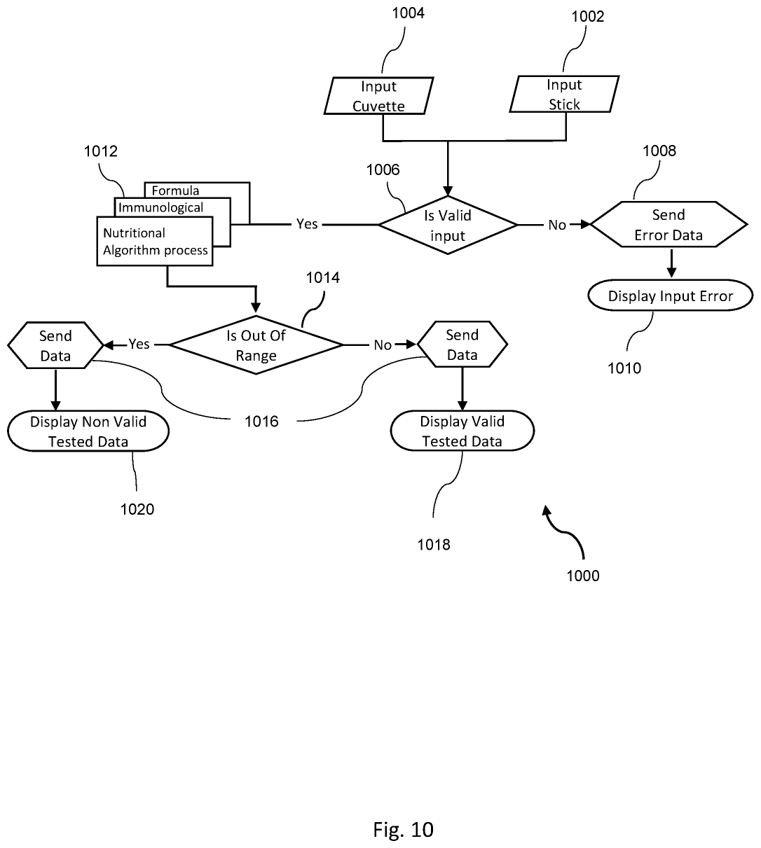

Reference is now made to FIG. 10 which illustrates an algorithm of activities and/or actions 1000 of the system of the present invention, in accordance with some demonstrative embodiments.

As shown in FIG. 10, algorithm 1000 may be fully automatic and computerized, except for the initial stage in which the user is required to choose the method by which the sample is inserted into the device (dipstick input 1002 or cuvette input 1004).

According to some embodiments, algorithm 1000 may include a checkpoint 1006 that gives an indication about the correctness of sampling placement into the device. In a setting, an error message 1008 can be displayed on the device's screen and/or transmitted to an external device (1010).

According to some embodiments, algorithm 1000 may have predefined processes for analyzing the composition of the sample being tested. According to some embodiments, these processes 1012 enable to analyze the nutritional and/or immunological composition of the breast milk sample, and may, for example, determine the appropriate formula for the infant and/or alert regarding the possible development of a disease.

According to some embodiments, algorithm 1000 may include a checkpoint 1014 for detecting abnormal deviations in the values of the sample's components being tested. According to some preferred embodiments, these deviations may be identified by comparing the sample to some reference values.

For example, referring to the horizontal stick—Zone 3 may act as a control zone, which confirms that the test is working properly. According to some embodiments, in zone 3, the antibody/antigen/enzyme which travels from Zone 1 should activate a dye, regardless of the presence of the bound molecule (i.e. the target molecule). This is to ensure the antibody/antigen/enzyme has been properly detached from Zone 1, has travelled across the stick, and the enzyme/conjugate/metal/color it carries which causes the color reaction/development/accumulation is functioning properly.

For the dipstick, there are a few possible scenarios for the assessment of false results:

a. a control square with a generic test which should always be positive, that indicated the stick is valid and the milk sample has been properly introduced.

b. each test square could contain a two-toned reaction, one would indicate a positive result (a match with the target molecule) and the other would be a negative result (no match with the target molecule).

c. each square might have two layers, similar to the horizontal stick, with the outer layer acting like Zone 3. In addition, the outer layer might induce a different color in case of a negative result.

According to some embodiments, algorithm 1000 may enable displaying the results of the analysis on the device's screen, whether valid (1018) or non-valid (1020) results, and/or sending these data to external device (1016).

According to some demonstrative embodiments the system of the present invention may include one or more of the following: sensors that can translate the results from the nutritional/immunological tests to easy-to-understand parameters; Bluetooth component that communicates with a smartphone application; Separately-purchased box with 30 disposable nutritional stick and 1 disposable immunological stick; Nutritional stick allows for the monitoring of the nutritional composition of the breast milk (including total antibodies concentration); Immunological stick checks for the presence of specific childhood diseases; an application to help mothers monitor their infants' growth, health and development.

According to some embodiments, the application may include the following features: User Friendly; Alerts the mother when her breastmilk shows deviations from her infant's nutritional needs; Gives nutritional suggestions that can improve the breast milk quality; Alerts the mother for possible illnesses in her infant; Gives invaluable nutritional and immunological information in case of a visit to the doctor; Growth monitoring section where the mother can input data and keep track of her infant's development.

According to some embodiments, the system may integrate with Child Care Centers and/or Human Milk Banks.

According to some embodiments, a nursing mother using the system of the present invention may perform a daily nutritional test and discover that her milk lacks essential fatty acids. The application of the system may notify her and can throughout the day send her reminders to eat a variety of foods that contain these lacking ingredients.

According to some embodiments, for mothers who choose to mix formula feeding with breast feeding, relying on the nutritional information from the nutritional test performed by the mothers, the application can suggest which stage of the formula best fits their infant (stage 1, stage 2 or stage 3). This way the mother can choose a formula that best meets her infant's developmental needs.

According to some embodiments, the regular nutritional stick sampling element may also contain a test for general antibody concentration in the breastmilk. As soon as the test shows an abnormal result, the application will let the mother know she should use an immunological stick to provide a more accurate analysis of the breast milk.

According to some embodiments, an abnormal antibody concentration in the breastmilk suggests that the infant might get sick in the following days.

According to some embodiments, the immunological kit tests for common childhood diseases. If the test is positive for any of the diseases, the application will let the mother know, suggest she should check her child's fever and ask whether she wants to make a doctor's appointment. According to some embodiments the system of the present invention may allow for a prediction of a baby's disease, thereby obviating the need to see a physician. For example, a baby in his first year of life visits the doctor at least nine times. The system of the present invention and the application thereof will give the mother the tools necessary to predict possible illnesses and give the doctor critical information, be it the results of the immunological kit or the fever monitoring which was recorded by the mother.

According to some embodiments, the application will have a dedicated section where the mother can input data regarding her infant's growth, development, mood/behavioral changes, and so on. The mother will be able to use this information to track her infant's growth and development, as well as pick up behavioral patterns.

For example, the mother inputs her infant's weight at every weighing with the doctor/at infant care centers. The application will save this information for her convenience and will also be able to show her in graph form her infant's growth through.

According to some embodiments, the system may integrate with Child Care Centers:

1. The mother's breastmilk's nutritional and immunological information is saved over time in the device's application, and can be used to monitor the child's growth and development. Child Care Centers can use that information to make sure that the infant is receiving the adequate nutrition for his age. If, for example, the infant is under the normal weight for his age, the nurse at the care center can look over the mother's nutritional information and make suggestions accordingly.

2. The mother can input data from the care center check-ups such as the baby's weight and head circumference in the application's growth monitoring section, so that all her infant's growth data is organized in one place, in an easy and accessible manner.

According to some embodiments, the system may integrate with Human Milk Banks.

There are Human Milk Banks in about 33 countries around the world. These are multi-million dollar establishments on which countless infants rely as their only source of human breast milk. Our device can be used by the Human Milk Bank to determine the nutritional quality of the milk they collect or receive, see whether it meets the required criteria and classify it accordingly.

According to some embodiments, the system of the present invention may include specific sampling elements, with correlation to the specific region the breastfeeding mother is located. For example, a stick that check specifically for region-based diseases (either occurrence in the mother that may infect the child through breastmilk or occurrence in the infant). For example, a stick that checks for the Zika virus or for HIV presence in the breastmilk.

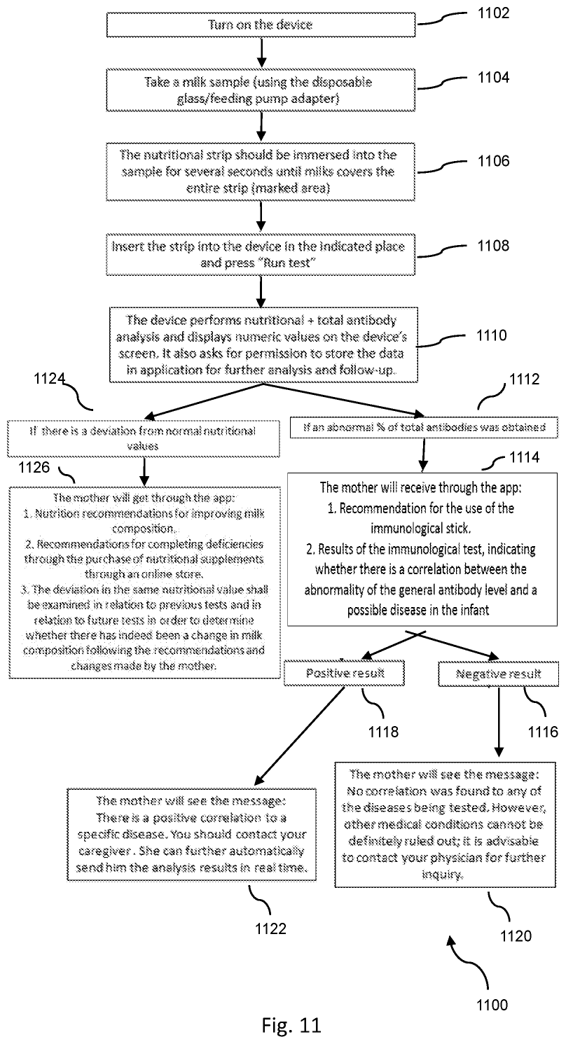

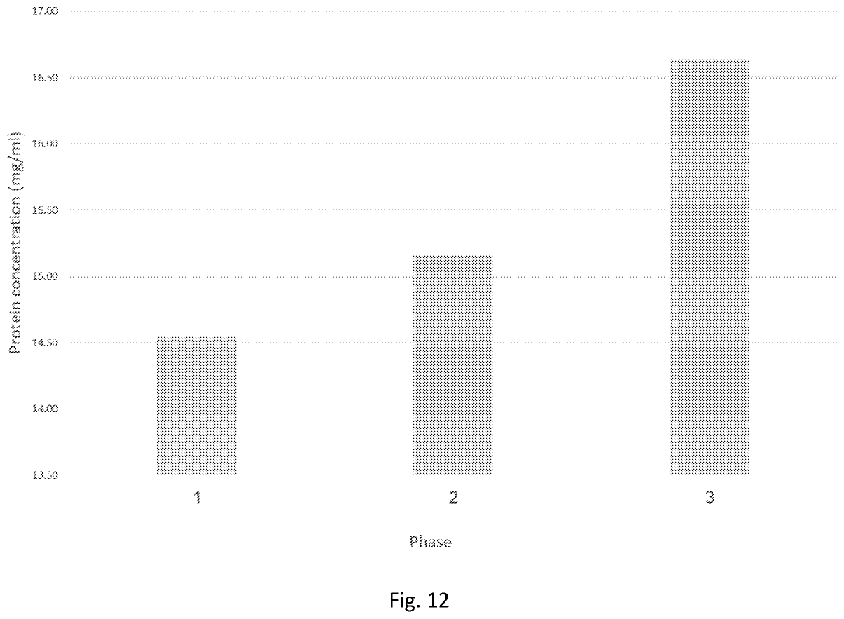

Reference is now made to FIG. 11, which is a flow chart of the operation 1100 of the system of the present invention, in accordance with some demonstrative embodiments.

As described in FIG. 11, the system's operation may include the following steps: turning a device of the system (1102) by a power switch;

taking a breast milk sample 1104, for example, using either a disposable glass or a feeding pump adapter (both of which may come along with the system);

dipping the nutritional strip in the milk sample 1106;

inserting the stick into the device in its indicated place and pressing a dedicated key to run the test 1108.

According to some embodiments, the system further may perform an analytical step 1110 of nutritional and total antibody status, at the end of which it presents numerical results on the device's screen. According to some embodiments, this step includes an option to store the data in the application for future use.

According to some embodiments and as described in FIG. 11, the system may identify an abnormal status 1112 of total antibody level, and accordingly send the user one or more notifications 1114 through the application. According to some preferred embodiments, these notifications may come in the form of text and may include a recommendation to use the immunological stick. In such a case, the system may test the milk sample for specific antigens (e.g. viruses, bacteria and the like). Eventually, the system may perform a correlation between the immunological test results and the risk of the infant to acquire a disease.

According to some embodiments and as described in FIG. 11, the results of the immunological test may be presented to the user as a message via the application. According to some embodiments, this message may come in the form of text.

According to some preferred embodiments, in case of negative result 1116, the user may receive a message 1120 informing that there was no correlation found between total antibody test and any of the diseases test, although other medical conditions cannot be totally ruled out and a further inquiry is recommended with her physician.

According to some preferred embodiments, in case of positive result 1118, the user may receive a message 1122 informing that a correlation to a specific disease was indeed found, along with a recommendation to visit her physician for further inquiry.

According to some embodiments and as described in FIG. 11, the system may identify an abnormal status 1124 of nutritional components in the milk, and accordingly send the user one or more notifications 1126 through the application.

According to some embodiments, these notifications may include nutritional and/or dietary recommendations aimed to improve breast milk composition as a function of the nutritional test results. According to some embodiments, the system may allow a comparison between the present test values and previous tests stored in the device and/or application, allowing a continuous follow-up and recommendations for specific formulas for partially breastfed infants.

According to some embodiments, the device of the present invention may include a computer having a pre-defined algorithm, wherein the algorithm may compare the test results of a user to the user's previous results, as well as to the mean results of the population using the device and data obtained through literature and/or any other suitable database.

According to some embodiments, there is provided herein a quick test kit enabling partially lactating mothers to monitor the development of their babies and to decide when to move to another stage formula.

According to these embodiments, upon analyzing the composition and/or concentration of several components in the mother's breast milk, the system of the present invention allows for the determination of the development stage of the infant. According to some embodiments, the system may be suitable for all breastfeeding mothers, including for example, mothers to infants who are solely breastfed, wherein the system may provide nutritional and/or immunological recommendations relating to the infant and/or mother; or to mothers to infants which have a combined nutrition, i.e., are both breastfed and fed by supplemental formulas, wherein the system may provide a recommendation for a specific supplemental formula for the individual needs of the infant regardless of the infant's age.

According to some demonstrative embodiments, there is provided a method for establishing an infant's nutritional needs and enabling a care provider to decide if the infant is ready for the next phase formula, for example, not just according to an average aged based but individually. According to some demonstrative embodiments, the method of the present invention may include measuring the maternal milk which reflects the condition of the infant.

One simple example is the first 3 distinctive stages in maternal milk: colostrum, transitional milk, and mature milk. According to some examples, the first formula is for very young babies that need the colostrum or the transitional milk (usually between 0-1 months). The second formula may be used for babies that need mature milk, and the third formula is intended for mature infants, usually over 1 year old.

According to some embodiments, there are provided herein infant formulas which may be divided to different content according to the developmental needs of the growing infant. For example, the formulas may include 6 different formulas that address most of the infant's needs and other formulas that are more specific to different conditions.

According to some embodiments, the kit of the present invention may include a cartridge with a funnel that ensure that a set volume of maternal milk (e.g., 50 μl) will be measured.

According to some embodiments, the kit may be based on an enzymatic, biological and/or chemical reactions. The kit is based on a algorithm that can factor in the different tests and to recommend on the next steps that should be taken.

For example, the kit may enable to determine that the total protein of a mother's milk is changing with time postpartum. According to some embodiments, different formulas may include different total protein levels. According to some embodiments, a mother using the kit of the present invention may test her milk, and according to the total protein levels of the milk, the kit of the present invention may indicate to the mother which of the existing formulas possesses a total protein level which is closest to the actual total protein of the mother's milk.

### Example 1

1. The mother will put maternal milk up to a line in the cartridge (about 5 ml).

2. After 5 minutes there will be a development of a color (e.g. blue). The kit will come with a scale of the color (e.g. light to dark blue) corresponding to the different formulas or stages. The funnel will direct the milk sample to the other tests as well.

### Example 2

The mother will inject a requested volume (1 mL) into a container with copper sulfate and 1% (w/w) of sodium hydroxide. The liquid in the container will turn purple and by comparison of the observed shade to the shade scale given (maximum 5 shades), the protein concentration range is known.

### Example 3

A colorless, transparent, semi permeable, perforated tube containing phosphomolybdate and phosphotungstate, is inserted inside a sample of maternal milk (minimum 5 mL). After 3 seconds of immersion, the tube is removed from the maternal milk and a shade of the color blue is formed inside the test tube. The observed shade is compared to the shade scale provided with the kit to find the protein concentration of the maternal milk that was tested.

### Example 4—Antibodies

In our case, the nutritional strip/dipstick will have an antibody for CD45 in order to monitor an increase that is in high correlation to an infection. Thus, may indicate that the baby is becoming sick, or that the mother is becoming sick. The antibodies will bind to the CD45 as described (liberation pad) and the intensity of the signal could be measured either by comparison to a color-panel as in alternative 1 or with a sensor in a designated device.

If the count is high, the application will recommend the mother to use the immunological strip/dipstick. Once the immunological strip will indicate a problem, the application will guide the mother for further actions including a recommendation to visit the physician.

### Example 5—Enzymatic Response

Glucose Test

The mother's milk is rich with glucose, however, monitoring its levels might be important for the baby growth especially if there are great changes on the same conditions. Clearly, there will be differences in the glucose levels before and after a meal. However, if the mother is measuring the glucose levels under the same conditions it should be more or less the same. The detection of glucose by test strips is based on the enzymatic reaction of glucose oxidase. This enzyme catalyzes the oxidation of glucose by atmospheric oxygen to form D-glucono-δ-lactone and hydrogen peroxide. A second linked reaction, mediated by a peroxidase, catalyzes the reaction between the peroxide and a chromogen (a substance that acquires color after a chemical reaction) to form a colored compound that indicates the glucose concentration.

1) Catalyzed by glucose oxidase:

Glucose+O₂→D-glucono-δ-lactone+H₂O₂

2) Catalyzed by peroxidase:

H₂O₂+Chromogen→Oxidized chromogen (colored)+H₂O

### Example 6—Color Response

Protein concentration is important for the nutrition of the growing baby. Therefore, the nutritional strip will measure the protein levels. One of the ways to measure the protein level is by utilizing the principle of Bradford assay. This assay involves the binding of Coomassie Brilliant Blue dye to proteins. Under acidic conditions, the dye is predominantly in the doubly protonated red cationic form (Amax=470 nm). However, when the dye binds to protein, it is converted to a stable unprotonated blue form (Amax=595 nm). Changes in protein concentration can be visualized by utilizing the Coomassie by naked eye as well. The application would be able to keep record on the protein concentration and to recommend the mother on a diet or the most suitable formula for the baby.

### Example 7

Detecting CMV antibodies (CMV-IgG and CMV-IgM), especially important for pre-term babies who have not received the mother's CMV immune factors. Cytomegalovirus is usually not a deadly virus among healthy infants and adults, but can be dangerous for pre-term babies. The virus can lie dormant or appear as other common diseases. Early detection through the presence of antibodies in the breast milk can help prevent the virus from developing in the infant into a dangerous disease.

### Example 8

Human Oligosaccharides (HMOs) are lipids which feed the infants' intestinal fauna (the bacteria within the infant's digestive system). An imbalance in HMOs (a decrease or increase from the Healthy concentrations—Colostrum: 20-25 g/L and Mature Milk: 5-20 g/L) may lead to an imbalance in the infant's digestive system, which can cause cramps, diarrhea, gas etc.’. Detecting an imbalance of HMOs as the cause for the infant's discomfort can help the mother take early and quick measures to ensure an increase/decrease in HMOs in her breastmilk (through changes in her eating habits) and help her infant recover.

### Example 9

Elevated levels of IL-6 have been observed when a pathogenic bacteria (such as Bacterial Meningitis) takes root. The healthy level of IL-6 in colostrum is 978.80±86.80 pg/mL, in transitional milk it is 162.90±29.67 pg/mL and in mature Milk it is 86.92±2.47 pg/mL according to Ustundag, Bilal, et al. By diagnosing elevated levels of IL-6, the parents of the infant can be forewarned of a possible bacterial infection at play and an early detection and prevention of the infection may be achieved.

While this invention has been described in terms of some specific examples, many modifications and variations are possible. It is therefore understood that within the scope of the appended claims, the invention may be realized otherwise than as specifically described.

### Example 10

Protein concentration and estimated the carbohydrates quantity in breast milk were determined and compared between 6 groups according to the age of the infant.

Samples collection—Breast milk samples were collected from 98 Israeli donors whose infants' ages ranged from 7 days to 843 days, about 16 donors from each age group. The infants were divided to age groups: 0-1 months, 1-3 months, 3-6 months, 6-9 months, 9-12 months and 12 months and above.

The tested infant formulas were the three main brands in the Israeli market: Materna, Similac and Nutrilon.

Protein quantification—The protein concentration was determined by two methods: Bradford assay (Bio-Rad Protein Assay) and BCA assay (Pierce™ BCA Protein Assay Kit), both are spectrophotometric methods using ELISA kits. Bradford assay was independently executed both by M.A.O FoodTech Labs and by the Technion.

Carbohydrates quantification—Thermo Scientific's Glycoprotein Carbohydrates Estimation Kit. A spectrophotometric method using ELISA kit.

Results:

First, the protein concentration of three different infant formulas in the market was assessed. These (and many other) companies divide the infant's development into 3 stages or phases: phase 1 (0-6 months), phase 2 (6-12 months) and phase 3 (1 year+). In FIG. 12 the protein concentrations of the three-phase products of Nutrilon® are presented. It can be noticed that there is an increase in the protein content between phase 1 to 3, ranging from 14.5 g/ml in phase 1 to 15.1 g/ml in phase 2 and 16.6 g/ml in phase 3.

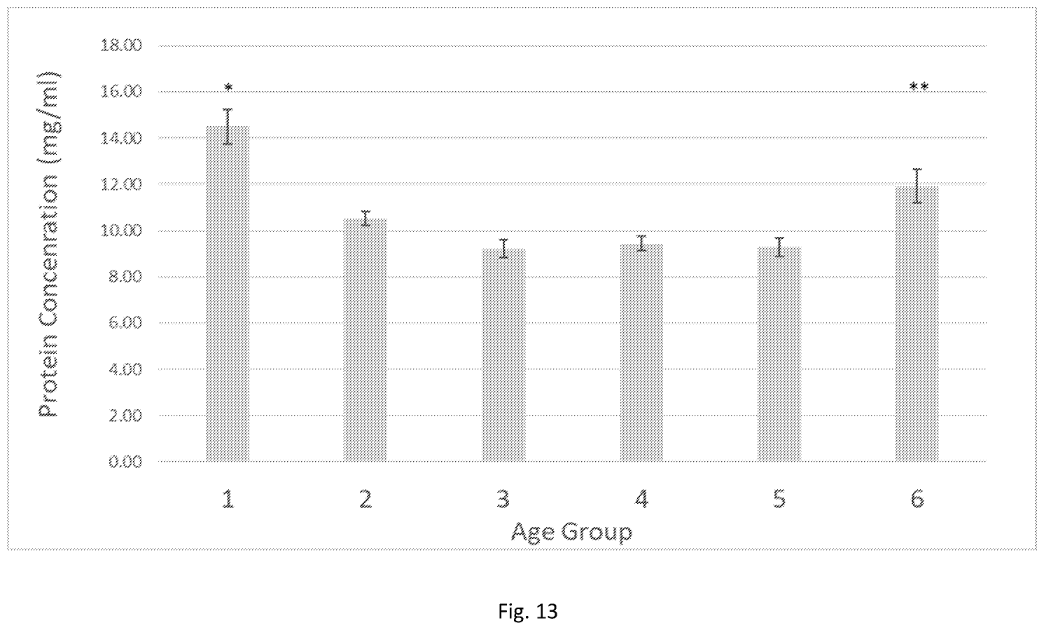

Next we wanted to quantify the protein content in human breast milk. We divided the donors into 6 groups, according to the age of the infant. Using Bradford and BCA assays, we quantified the average protein concentration in each of the groups. As can be seen in FIG. 13, (which shows graph depicting the protein concentrations of the human milk at different age groups, using Bradford assay. Graphs show mean and S.E values, n=99; * indicates statistical significance (P<0.05) to all other age groups, and ** to all other age groups except for group 2) the highest average protein concentration can be found in group 1 (14.5 mg/ml). In group 2 there is a major decrease in the protein concentration (10.5 mg/ml), and in groups 3 a further moderate decrease, which stays steady until group 5 (9.2-9.4 mg/ml). In groups 6 there is again an increase in the protein concentration (11.9 mg/ml). In addition, there was further increase after 15 months, which cannot be seen in the graph. There was no pattern within each age group.

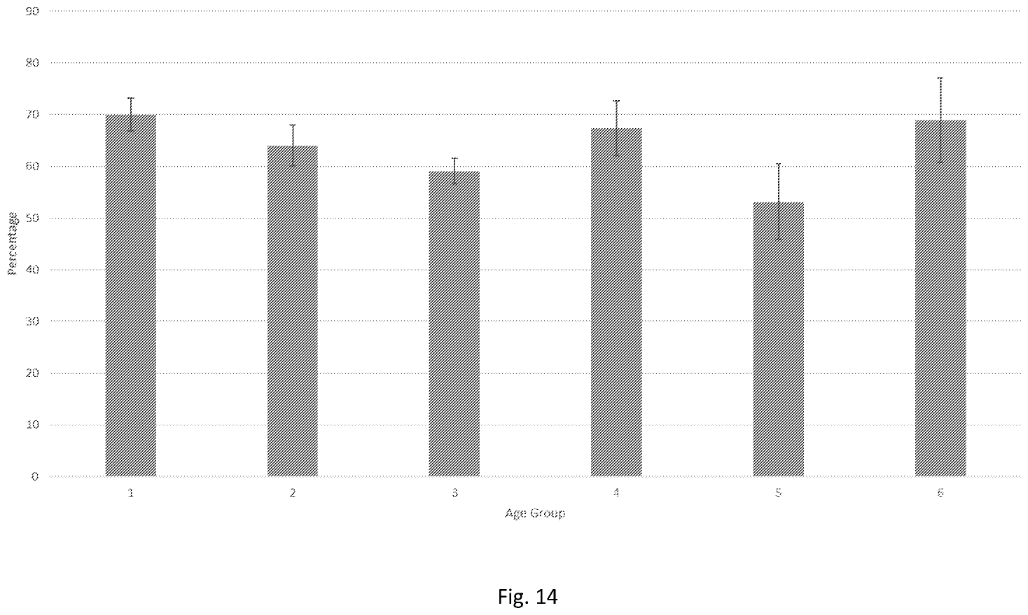

Carbohydrates found in the breast milk were also estimated. Thus, we found the percentage of carbohydrates in glycoproteins in the breast milk, in a known concentration of protein (0.25 mg/ml). The absorbance was outside of the linear range of the calibration curve. Therefore, we normalized the absorbance into a relative percentage of the carbohydrates content in reference to the blank (0%) and to the maximal absorbance. As shown in FIG. 14, there is no clear pattern when comparing the age groups. In addition, most of the results are not statistically significant from one another (P>0.05). In addition, there is high variance within each age group between the mothers.

When comparing FIG. 12 and FIG. 13, major differences between the two trends in the protein concentration along the development of the infant is seen. Not only does the formulas roughly divide the infant's development into three stages, but it also suggests protein values that are different from those found in real breast milk. While there is a major increase in the formula, our results show that the protein in breast milk decreases until age 3-12 months, and increases afterwards. Both groups 1 and 6 are statistically significant (P-value<0.05), a result that emphasize that this trend in protein concentration is real.

The high protein concentration in the first month adheres to our assumption and can be explained by the high immunologic factors present in the milk after giving birth. The decline after the first month also corresponds with the literature. The increase in protein after 12 months however is surprising and may suggest that there is a change in the protein composition in the milk, which is necessary for the infant.

These results were analyzed using the fast Bradford ELISA kit, as well as BCA ELISA kit. Both assays gave similar results, and also the two independent experiments with Bradford method gave similar results. This finding makes our results more robust. In addition, Bradford method is quick to execute and gives fast result. This allows us to repeat and further continue our research easily. The results reflect averages of the protein concentration and there was high variation between the donors. This finding also supports the fact that each baby is nourished by different nutrient composition, unlike the relatively uniform composition found in the formulas. The different milk composition can be either a result of the mother's or the infant's status.

In spite of the fact that the carbohydrates quantification assay did not give absolute values, it is clear from FIG. 14 that there is no clear pattern between the age groups. This can be derived from the high variance between the mothers. The infant formulas however suggest products with very minor changes in the carbohydrates content and do not correspond to the breast milk and subsequently to the biological age and needs of the baby.

In agreement with our assumption and the literature that the quantity of nutrients in maternal milk changes along the growth of the baby, we have seen differences in protein and carbohydrates levels in maternal milk. In correspondence to the literature, our results have indicated a decrease in total protein after the first month. However, our results have shown an increase in total protein after a year. There was a certain variation in total protein among mothers within the same age group and there was no trend within the group. In addition, there is high variance in the carbohydrate levels within each group.

These results are in conflict with the composition of the infant formulas as exist in the market today, which suggest more uniform products with little variance and no adherence to the biological age or status of the infant. Therefore, upon knowing the breastmilk composition, each mother will be able to choose a formula that fits better to her baby. Alternatively, more accurate and fine formulas can be developed, that will better fit the baby's status.

### Example 11

Infants require a different combination of proteins, lipids and sugars during the different stages of their growth. The human breast milk composition changes over time to fit the needs of the infant. For example, newborns require a higher concentration of protein and immunogenic factors, as opposed to older infants who require a higher concentration of lipids and energy. When developing an infant food supplement, the requirements of infants during their different developmental stages must be taken into account—one supplement will not fit all infants of all ages. This is why companies nowadays tend to create a three-stage model—three products in the same line that are designed for three developmental stages of the baby.

The purpose of this experiment comes in three parts: the first is to test whether there is a pattern in the amount of proteins and glycoproteins within human breast milk that corresponds to, or changes with, the age of the infant. The second is to analyze the differences in three infant food supplement brands using biological and chemical essays. And the third is to test simple kits for the identification of different developmental stage of the baby. This essay focuses on the quantification of proteins and carbohydrates (through glycoproteins quantification).

Human breast milk was collected separately from this protocol, and divided into six groups according to the infant's age: 0-3 months, 3-6 months, 6-9 months, 9-12 months and 12 months and over. 90 donors participated, 15 donors from each age group. 90 samples for the use of this experiment were collected.

The infant food supplements used in this experiment is chosen from the three main brands in the Israeli market today: Formula 1 (Materna®), Formula 2 (Nutrilon®) and Formula 3 (Similac®). Each of these three has at least one line of products with a three-stage model. The three-stage model for all three supplement brands used in this experiment follows the following: Phase 1 is for infants between the ages of 0 to 6 months, Phase 2 is for infants between the ages of 6 to 12 months, and Phase 3 is for infants over the age of 12 months. The tests performed in this essay included:

Bradford: based on an absorbance shift of the dye Coomassie Brilliant Blue G-250. The dye changes its hue from red to blue when in contact with an acid, and binds to the proteins in the liquid it has been placed. According to the book Nutrition During Lactation, Chapter 6: Milk Composition, total protein concentration decreases rapidly during the first month post-partum, and keeps decreasing gradually for the next five months (results were not shown beyond the first five months after birth) (Lactation, 1991). Therefore, we expect the total protein concentration to be higher in Phase 1 formula than in Phase 2 formula and so on.

BCA: An assay based on the bicinchoninic acid (BCA) for the colorimetric detection of total protein concentration. This method combines the reduction of Cu+2 to Cu+1 by protein in an alkaline medium with the colorimetric detection of the cuprous cation (Cu+1) using a reagent containing bicinchoninic acid. The purple-colored reaction product of this assay is formed by the chelation of two molecules of BCA with one cuprous ion. This complex exhibits a strong absorbance at 562 nm. This reaction has a broad working range (20-2,000 μg/ml) (Sino Biological).

Glycoprotein Carbohydrate Estimation Kit: The Thermo Scientific Glycoprotein Carbohydrate Estimation Kit is used for the detection of carbohydrate content. Glycoprotein is first oxidized with sodium meta-periodate to form aldehydes that react with the proprietary Glycoprotein Detection Reagent. The resulting purple reaction may be detected at 550 nm. Unknowns are compared with protein standards of known glycoprotein content. It is important to note that non-glycosylated proteins, such as lysozyme and bovine serum albumin, produce a low absorbance at 550 nm.

All above molecular assays rely on the use of a standard with known concentrations of the molecule in question. With these standards it is possible to create curves where the x-axis represents the spectrophotometric readout, and the y-axis represents the concentration of the sample. In order to receive the most accurate results, it is required that the spectrophotometric result of the unknown sample falls within the linear part of the standard curve.

Objectives:

1. To test whether there is a pattern within human breast milk that corresponds to the age of the infant/changes with the age of the infant.

2. To quantify and graph the pattern in human breastmilk which corresponds to/changes with the age of the infant. The parameters tested for this pattern are: total protein concentration and total carbohydrates concentration.

3. To test whether there is a pattern within brand baby food supplements that matches the changes in human breast milk with correspondence to the age of the infant.

The human breast milk composition changes as the infant grows in age. These changes may be noted in decreased levels of proteins and increased carbohydrates (especially in the form of lactose and oligosaccharides). The composition data available on the sites of the brand baby food supplements suggest there exists a small pattern in increased levels of protein as the phases progress and increased levels of carbohydrates as the phases progress (with the notable difference of Similac®, that remains almost unchanged within the phases).

The projected concentrations of the values tested in this experiment are depicted in table 6 below:

TABLE 6
Concentrations of proteins and carbohydrates and fat within the nine-brand product, as
disclosed on each of the three brand's websites and packaging, according to the manufacturer's
instructions of preparation, using DDI water.

| | Materna | Materna | Materna | Nutrilon | Nutrilon | Nutrilon | Similac | Similac | |
|---|---|---|---|---|---|---|---|---|---|
| | Phase 1 | Phase 2 | Phase 3 | Phase 1 | Phase 2 | Phase 3 | Phase 1 | Phase 2 | Similac |
| | (0-6 | (6-12 | (12+ | (0-6 | (6-12 | (12+ | (0-6 | (6-12 | Phase 3 |
| | MO) | MO) | MO) | MO) | MO) | MO) | MO) | MO) | (12+ MO) |
| Protein | 16.50 | 17.25 | 18.00 | 14.55 | 15.16 | 16.64 | 15.58 | 29.41 | 31.96 |
| (Albomin/Casein | | | | | | | | | |
| 60/40) [mg/ml] | | | | | | | | | |
| Carbohydrates | 7.7 | 7.8 | 8.4 | 7.3 | 8.6 | 8 | 7.4 | 7.5 | 7.5 |
| [gr/100 ml] | | | | | | | | | |

TABLE 7
Projected range of values for proteins and carbohydrates and fats
in the infant food supplements. Based on information from table 1.

| | Range Phase | Range Phase | Range Phase |
|---|---|---|---|
| | 1 (0-6 MO) | 2 (6-12 MO) | 3 (12+ MO) |
| Protein (Albomin/Casein | 14.55-16.5 | 15.16-29.41 | 16.64-31.96 |
| 60/40) [mg/ml] | | | |
| Carbohydrates [gr/100 ml] | 7.3-7.7 | 7.5-8.6 | 7.5-8.4 |

Materials & Equipment:

TABLE 8
Complete materials and equipment list for all experiments.

| | |
|---|---|
| 1 | Materna Dairy Phase 1 |
| 2 | Materna Dairy Phase 2 |
| 3 | Materna Dairy Phase 3 |
| 4 | Similac Top Phase 1 |
| 5 | Similac Top Phase 2 |
| 6 | Similac Top Phase 3 |
| 7 | Nutrilon Phase 1 |
| 8 | Nutrilon Phase 2 |
| 9 | Nutrilon Phase 3 |
| 10 | 90 breast milk samples, aliquoted. |
| 11 | Eppendorf's tubes |
| 12 | Pipettes (1 mL to 5 mL, 5 μl to 100 μl, 100 μl to 1000 μl) |
| 13 | Pipette Tips for 1 mL to 5 mL, 5 μl to 100 μl and 100 μl to 1000 μl |
| | pipettes |
| 14 | Elisa reader (Spectrophotometer with a plate reader) |
| 15 | 96 well plates |
| 16 | 10 250 ml glass beakers |
| 17 | 10 100 ml glass beakers |
| 18 | Distilled water |
| 19 | Bovine Serum Albumin (BSA) |
| 20 | Bradford Dye Reagent Concentrate |
| 21 | Markers (for marking Eppendorf's Tubes) |
| 22 | Digital Scales |
| 23 | Measuring spoon |
| 25 | BSA (protein Standard) |
| 24 | Stopper |
| 25 | Rhenium BCA Protein Assay Kit |
| | ->BCA Reagent A |
| | BCA Reagent B |
| | ->Albumin Standard Ampules, 2 mg/mL |
| 26 | Multichannel pipette |
| 27 | Thermo Fisher Scientific's Glycoprotein Carbohydrates |
| | Estimation Kit |
| | Sodium meta-periodate, 500 mg |
| | Glycoprotein Detection Reagent, 500 mg |
| | Glycoprotein Assay Buffer, 250 mL, contains 0.1% sodium azide |
| | Negative Controls: lysozyme, 2.5 mg; bovine senim albumin, 2.5 mg |
| | Positive Controls: ovalbumin, 2.5 mg; human apotransferrin, 2.5 mg; |
| | fetuin, 0.25 mg; α1-acid glycoprotein, 0.25 mg |
| 28 | −20° C. Storage space |
| 29 | −80° C. Storage space |
| 30 | Incubator (37° C.) |
| 31 | 96 well plate Shaker |
| 32 | Vortex |
| 33 | Bucket |
| 34 | Ice |
| 35 | Stands for Eppendorf's tubes |
| 36 | Electric dispenser |
| 37 | Stirrer |
| 38 | Magnets |
| 39 | Breast Milk Samples |
| 40 | Heat Plate |
| 41 | Label Printer for Eppendorf's tubes labels |

Methods:

Part I: Sample Preparation, Dilutions and Testing BCA Incubation Time

Materials & Equipment:

TABLE 9
Materials and Equipment needed for sample preparation and dilutions.

| | |
|---|---|
| 1 | Materna Dairy Phase 1 |
| 2 | Materna Dairy Phase 2 |
| 3 | Materna Dairy Phase 3 |
| 4 | Similac Top Phase 1 |
| 5 | Similac Top Phase 2 |
| 6 | Similac Top Phase 3 |
| 7 | Nutrilon Phase 1 |
| 8 | Nutrilon Phase 2 |
| 9 | Nutrilon Phase 3 |
| 10 | Breast Milk Samples |
| 11 | Eppendorf's tubes |
| 12 | Pipettes (1 mL to 5 mL, 5 μl to 100 μl, 100 μl to 1000 μl) |
| 13 | Pipette Tips for l mL to 5 mL, 5 μl to 100 μl and 100 μl to 1000 μl |
| | pipettes |
| 14 | Markers (for marking Eppendorf's Tubes) |
| 15 | Digital Scales |
| 16 | Measuring spoon |
| 17 | 10 250 ml glass beakers |
| 18 | 10 100 ml glass beakers |
| 19 | 3 96 wells plate |
| 20 | Elisa reader (Spectrophotometer with a plate reader) |
| 21 | Incubator (37° C.) |

Step 1: Discovering the correct dilutions:

For the Powder Supplements:

The dilutions used: 1:50, 1:100, 1:200, 1:400, 1:800

Tests were done in TRIPLICATES

For Materna® Dairy Phase 2, prepare a batch of the product according to the manufacturer's instructions (Table 5) using DDW water (60 ml). this is the 1:1 batch. Use magnetic stirrer until mixture is homogenous

TABLE 10
The manufacturer's instructions for the
preparation of the formulas, made with DDI water.

| Formula + Phase | gram | ml |
|---|---|---|
| Nutrilon Phase 1 | 9 | 60 |
| Nutrilon Phase 2 | 4.9 | 30 |
| Nutrilon Phase 3 | 4.8 | 30 |
| Materna Phase 1 | 9 | 60 |
| Materna Phase 2 | 9 | 60 |
| Materna Phase 3 | 9 | 60 |
| Similac Phase 1 | 8.8 | 60 |
| Similac Phase 2 | 10.4 | 60 |
| Similac Phase 3 | 10.2 | 60 |

Step 2: Create the following dilutions: 1:1, 1:10, 1:100, 1:1000, 1:10000.

For 1 ml of the 1:10 dilution, take 100 μl of the 1:1 batch, and add 9000 μl of distilled water. Vortex thoroughly. Do the same to create the next dilution.

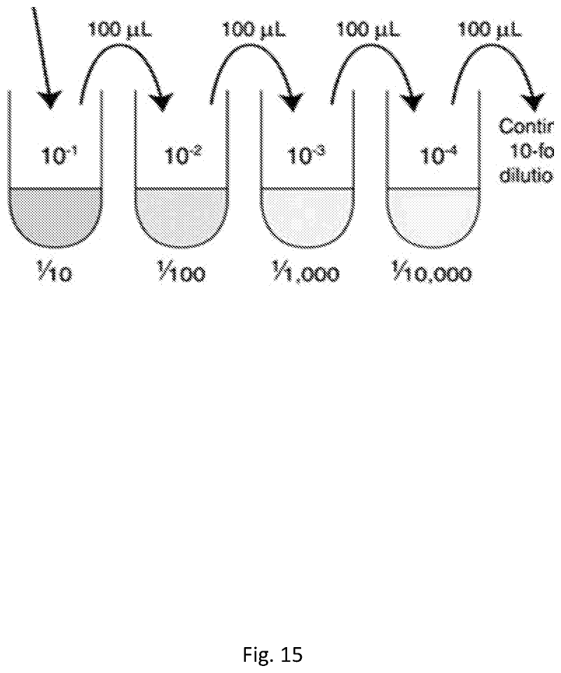

See as referenced in FIG. 15.

2. Label each sample as such:

TABLE 11
Sample labeling guidelines, where XX is
the dilution number (i.e. 1:1, 1:1000).

| | |
|---|---|
| Materna Dairy Phase 1 | 1.A XX |
| Materna Dairy Phase 2 | 1.B XX |
| Materna Dairy Phase 3 | 1.C XX |
| Similac Top Stage 1 | 2.A XX |
| Similac Top Stage 2 | 2.B XX |
| Similac Top Stage 3 | 2.C XX |
| Nutrilon Stage 1 | 3.A XX |
| Nutrilon Stage 2 | 3.B XX |
| Nutrilon Stage 3 | 3.C XX |

3. Preform the following assays using all seven dilutions:
Bradford Assay
Carbohydrates Assay

4. To choose the dilutions: the dilutions that fall into the linear part of the concentration graph are those chosen. Five dilutions in total. If needed, repeat step 1 with different dilutions in order to find the best fit for the assays.

For the Human Breastmilk: Make the dilutions on the basis of the dilutions found for the formula. Otherwise, the following dilutions are used: 1:1, 1:10, 1:100, 1:1000, 1:10,000. Tests are done in triplicates
1. The samples “as is” will serve for the 1:1 dilutions.
2. For the 1:100 dilution, take 10 μl of the 1:1 sample (the sample “as is”) and add 990 μl of distilled water. For the 1:1000 dilutions, take 100 μl of the 1:100 dilution and as 9000 μl of distilled water.
3. Label the human breast milk dilutions by using the code on the sample's Eppendorf's tube and add the dilution (i.e. 1:1, 1:100 or 1:1000).
4. Preform the following assays for one sample from each of the six breast milk groups (0-3 months, 3-6 months, 6-9 months, 9-12 months and 12 months and over):
   Bradford Assay
   Carbohydrates Assay.
5. From the results, determine which dilutions might fall into the linear part of the protein concentration graph. Five or four dilutions in total should be chosen.

Part II: Tests/Assays

The following tests and assays were be performed as follows:
1. Each test is done in triplicates.
2. For each test, there are Blanks and Standards (also in triplicates).
3. For each test there are the number of dilutions set forth in the Quick and Dirty test.
4. Note on Breast Milk:
   a. Number of Eppendorf's tubes used: Two Eppendorf's tubes per sample (tubes #4 and #10) were mixed. For some samples, tube #5 was used as well.
   b. Number of samples: Each “batch” to be tested will contain one (1) sample per age group, six (6) age groups, total of six (6) samples per “batch”. Number of batches tested is determined per day by the discretion of the team leader of the day.
   c. One sample from each of the six age groups is tested at a time, so that in total we have six samples being tested together, on the same 96-well plate (at least one “batch” per 96-well plate).
   d. To thaw the breast milk: Place Eppendorf's tubes on ice and let thaw. You may use a vortex or the heat from your fingers to quicken the process (only if the process for thawing is repeated exactly the same way for each of the 60 test tubes).

Bradford Assay

The purpose of this assay is to measure the total concentration of proteins in the samples and deduce whether there is a correlation between the changes in total protein concentration and the developmental stage of the infant. It is expected that the protein content of each products will increase as the developmental stages progress. The protein concentration for phase 1 will remain about the same for all three brand products. In phases 2 and 3 the protein concentration in Similac is the highest among the three brand products, while Materna will have the second highest concentration.
This test is performed twice—once on the powder infant food supplements and once on the breast milk samples.

The projected values and ranges for the total concentration of proteins in each of the samples are:

TABLE 12
based on Table 2 **These ranges refer to the 1:1 dilution.

| | Range Phase | Range Phase | Range Phase |
|---|---|---|---|
| | 1 (0-6 MO) | 2 (6-12 MO) | 3 (12+ MO) |
| Protein (Albomin/Casein | 14.55-16.5 | 15.16-29.41 | 16.64-31.96 |
| 60/40) [mg/ml] | | | |

Materials & Equipment:

TABLE 13
Materials and Equipment needed for Bradford Assay.

| | |
|---|---|
| 1 | infant formula samples in their dilutions (9 products × 5 dilutions) |
| 2 | Breast Milk Samples |
| 3 | BSA (protein Standard) |
| 4 | Elisa reader (Spectrophotometer with a plate reader) |
| 5 | 7 96-well plates |
| 6 | Eppendorf's tubes |
| 7 | Bradford Dye Reagent Concentrate |
| 8 | Multichannel pipette |
| 9 | Pipettes (1 mL to 5 mL, 5 μl to 100 μl, 100 μl to 1000 μl) |
| 10 | Pipette Tips for 1 mL to 5 mL, 5 μl to 100 μl and 100 μl to 1000 μl pipettes |
| 11 | Dark bottle or 250 ml glass beaker and aluminum paper |
| 12 | Stopper |
| 13 | Markers |

Procedure

TABLE 14
Amount of samples/product needed for this part.

| | Volume of | | Total volume of |
|---|---|---|---|
| Type of product | product | Replications? | product needed |
| Protein Standard | 10 μl | 3 | 30 μl |
| Sample | 10 μl | 3 | 30 μl |
| (breastmilk/supplement) | | | |
| Diluted Reagent (1:5) | 200 μl | 3 | 600 μl |
| Total (in each well) | 210 μl | | |

Total number of standards: 5 dilutions in triplicates (5×3=15)

Total number of blanks: 1 blank in triplicates (1×3=3) Total number of Powder Samples: 9 products in 5 dilutions in triplicates (9×5×3=135)

Total number of Breastmilk Samples: 5 samples from each of the six age groups, in five dilutions, in triplicates (5×6×5×3=450)
1. Prepare dye reagent by diluting 1 part Dye Reagent Concentrate with 4 parts DDI water. This diluted reagent may be used for about 2 weeks when kept at room temperature.
2. Prepare three to five dilutions of a protein standard, which is representative of the protein solution to be tested. The linear range of this microtiter plate assay is 0.05 mg/ml to approximately 0.5 mg/ml. Protein solutions are normally assayed in duplicate or triplicate.
   Dilutions for Standard (BSA): 0.75 mg/ml, 0.5 mg/ml, 0.4 mg/ml, 0.25 mg/ml, 0.1 mg/ml
   Dilutions for Powder Supplements: 1:20, 1:40, 1:80, 1:100
   Dilutions for Human Breast Milk: 1:20, 1:40, 1:80, 1:100
3. Pipet 10 μl of each standard and sample solution into separate microtiter plate wells.
4. Add 200 μl of diluted dye reagent to each well. Mix the sample and reagent thoroughly (30 seconds, medium speed) using a microplate mixer. Alternatively, use a multi-channel pipet to dispense the reagent. Depress the plunger repeatedly to mix the sample and reagent in the well. Replace with clean tips and add reagent to the next set of wells.
5. Incubate at room temperature for at least 5 minutes. Absorbance will increase over time; samples should incubate at room temperature for no more than 1 hour.
6. Mix for 5 seconds using the microplate mixer. Measure absorbance at 595 nm.

BCA Assay

An assay based on the bicinchoninic acid (BCA) for the colorimetric detection of total protein concentration (˜562 nm). The purpose of this assay is to measure the total concentration of proteins in the samples and deduce whether there is a correlation between the changes in total protein concentration and the developmental stage of the infant. It is expected that the protein content of each products will increase as the developmental stages progress. The protein concentration for phase 1 will remain about the same for all three brand products. In phases 2 and 3 the protein concentration in Similac is the highest among the three brand products, while Materna will have the second highest concentration.
This test is performed twice—once on the powder infant food supplements and once on the breast milk samples.

The projected values and ranges for the total concentration of proteins in each of the samples are:

TABLE 15
based on Table 2 **These ranges refer to the 1:1 dilution.

| | Range Phase | Range Phase | Range Phase |
|---|---|---|---|
| | 1 (0-6 MO) | 2 (6-12 MO) | 3 (12+ MO) |
| Protein (Albomin/Casein | 14.55-16.5 | 15.16-29.41 | 16.64-31.96 |
| 60/40) [mg/ml] | | | |

Materials & Equipment:

TABLE 16
Materials and Equipment needed for BCA assay.

| | |
|---|---|
| 1 | infant formula samples in their dilutions (9 products × 5 dilutions) |
| 2 | Breast Milk Samples |
| 3 | Rhenium BCA Protein Assay Kit |
| | ->BCA Reagent A |
| | ->BCA Reagent B |
| | ->Albumin Standard Ampules, 2 mg/mL |
| 4 | 7 96 well plates |
| 5 | Multichannel pipette |
| 6 | Pipettes (1 mL to 5 mL, 50 to 100 μl, 100 μl to 1000 μl) |
| 7 | Pipette Tips for 1 mL to 5 mL, 5 μl to 100 μl and 100 μl to 1000 μl pipettes |
| 8 | Incubator (37° C.) |
| 9 | Stopper |
| 10 | Elisa reader (Spectrophotometer with a plate reader) |
| 11 | Eppendorf's tubes |
| 12 | Markers |

Procedures:

TABLE 17
Amount of samples/product needed for this part.

| | Volume of | | Total volume of |
|---|---|---|---|
| Type of product | product | Replications? | product needed |
| Protein Standard | 25 μl | 3 | 75 μl |
| Sample | 25 μl | 3 | 75 μl |
| (breastmilk/supplement) | | | |
| Working solution | 200 μl | 3 | 600 μl |
| Total (in each well) | 225 μl | | |

Total number of standards: 5 dilutions in triplicates (5×3=15) Total number of blanks: 1 blank in triplicates (1×3=3)

Total number of Powder Samples: 9 products in 5 dilutions in triplicates (9×5×3=135)

Total number of Breastmilk Samples: 5 samples from each of the six age groups, in five dilutions, in triplicates (5×6×5×3=450)
1. Standard assay procedure
   Dilutions for Standard (BSA): 1 mg/ml, 0.75 mg/ml, 0.5 mg/ml, 0.25 mg/ml, 0.1 mg/ml
   Dilutions for Powder Supplements: 1:20, 1:40, 1:80, 1:100
   Dilutions for Human Breast Milk: 1:20, 1:40, 1:80, 1:100
   Prepare working solution
   Mix reagent A (in general the blue bottle in a BCA kit) and reagent B with ratio of A:B=50:1 for enough volume of using.
   Note: When Reagent B is first added to Reagent A, turbidity is observed that quickly disappears upon mixing to yield a clear, green WR. Prepare sufficient volume of WR based on the number of samples to be assayed. The WR is stable for several days when stored in a closed container at room temperature (RT).
2. Pipette 25 μL of each standard or unknown sample replicate into a microplate well (working range=20-2000 μg/mL).
3. Add 200 μL of the WR to each well and mix plate thoroughly on a plate shaker for 30 seconds. 4. Cover plate and incubate at 37° C. for 30 minutes.
5. Cool plate to RT. Mix for 5 seconds using the microplate mixer. Measure the absorbance at or near 562 nm on a plate reader.

Carbohydrates Assay

The purpose of this assay is to measure the total carbohydrates concentration in a liquid sample using Thermo Scientific's Glycoprotein Carbohydrates Estimation Kit and deduce whether there is a correlation between the changes in total Carbohydrates concentration and the developmental stage of the infant. It is estimated that the Carbohydrates concentration will increase for the Materna formulas as the developmental stages progress, while the Similac carbohydrates concentration will remain the same and the Nutrilon concentration will increase between stages 1 and 2 but decrease between stages 2 and 3. Materna will have the highest concentration, and Nutrilon the second highest, for phases 1 and 3, while for phase 2, Nutrilon will have the highest concentration, with Materna coming second.
This test is performed twice—once on the powder infant food supplements and once on the breast milk samples.

The projected values and ranges for the total concentration of carbohydrates in each of the samples are:

TABLE 18
based on Table 2 **These ranges refer to the 1:1 dilution.

| | Range Phase 1 | Range Phase 2 | Range Phase 3 |
|---|---|---|---|
| | (0-6 MO) | (6-12 MO) | (12+ MO) |
| Carbohydrates [gr/100 ml] | 7.3-7.7 | 7.5-8.6 | 7.5-8.4 |

Materials & Equipment:

TABLE 19
Materials and Equipment needed for carbohydrates assay.

| | |
|---|---|
| 1 | Infant formula samples in their dilutions (9 products × 5 dilutions) |
| 2 | Breast Milk Samples |
| 3 | Thermo Fisher Scientific's Glycoprotein Carbohydrates Estimation Kit |
| | Sodium meta-periodate, 500 mg |
| | Glycoprotein Detection Reagent, 500 mg |
| | Glycoprotein Assay Buffer, 250 mL, contains 0.1% sodium azide |
| | Negative Controls: lysozyme, 2.5 mg; bovine serum albumin, 2.5 mg |
| | Positive Controls: ovalbumin, 2.5 mg; human apotransferrin, 2.5 mg; fetuin, |
| | 0.25 mg; α1-acid glycoprotein, 0.25 mg |
| 4 | 7 96 well plates |
| 5 | Multichannel pipette |
| 6 | Pipettes (1 mL to 5 mL, 5 μl to 100 μl, 100 μl to 1000 μl) |
| 7 | Pipette Tips for 1 mL to 5 mL, 5 μl to 100 μl and 100 μl to 1000 μl pipettes |
| 8 | Incubator (37° C.) |
| 9 | Stopper |
| 10 | Elisa reader (Spectrophotometer with a plate reader) |
| 11 | Eppendorf's tubes |
| 12 | Markers |
| 13 | Microplate shaker |

Procedures:

TABLE 20
Amount of samples/product needed for this part.

| | Volume of | | Total volume of |
|---|---|---|---|
| Type of product | product | Replications? | product needed |
| Standard | 50 μl | 3 | 150 μl |
| Sample | 50 μl | 3 | 150 μl |
| (breastmilk/supplement) | | | |
| Glycoprotein Assay | 50 μl | 3 | 150 μl |
| Buffer (for blank) | | | |
| Sodium meta-periodate | 25 μl | 3 | 75 μl |
| solution | | | |
| Glycoprotein Detection | 150 μl | 3 | 450 μl |
| Reagent | | | |
| Total (in each well) | 225 μl | | |

Total number of standards: six standards in triplicates (6×3=18) Total number of blanks: 1 blank in triplicates (1×3=3)

Total number of Powder Samples: 9 products in 2 dilutions in triplicates (9×2×3=54)

Total number of Breastmilk Samples: 5 samples from each of the six age groups, in 2 dilutions, in triplicates (5×6×2×3=180)

Based on Thermo Fisher Scientific's Glycoprotein Carbohydrates Estimation Kit instructions manual.

Material Preparation

Note: Equilibrate the Glycoprotein Carbohydrate Estimation Kit components to room temperature before use.
1. Sodium meta-periodate Solution: Immediately before use, prepare 10 mM sodium meta-periodate by dissolving 21.4 mg of sodium meta-periodate in 10 mL of Glycoprotein Assay Buffer.
2. Glycoprotein Detection Reagent: Immediately before use, prepare 0.5% Glycoprotein Detection Reagent by dissolving 50 mg of the reagent in 10 mL of 1N NaOH.
3. Glycoprotein sample: Dissolve sample in Glycoprotein Assay Buffer at 0.25 and 2.5 mg/ml. If the sample is already in solution, dilute sample in Glycoprotein Assay Buffer at 0.25 and 2.5 mg/ml.
4. Protein standards During shipment, lyophilized proteins have may come in contact with the septa. Before opening, verify that protein is settled to the bottom of each vial. If necessary, gently tap the vial sides to settle protein. Carefully remove septa to avoid disturbing any protein that may have settled on its underside. Add 1 mL of Glycoprotein
   Assay Buffer to each protein standard vial. Replace septa and gently rock vial so that the buffer contacts all inside surfaces. Store reconstituted standard solutions for up to one month at 4° C.
After Protein Standards are reconstituted, aliquote each as such: 1700 μl of Standard into each marked Eppendorf's Tubes (leftovers in a final Eppendorf's tube, marked with an approximation of the liquid's volume)

Procedure
1. Place 50 μL of each standard and the sample in the plate wells. For the blank, use 50 μL of Glycoprotein Assay Buffer. Test each sample and standard in triplicate.
   Standards: Come with the kit, use as is.
   Dilutions for Powder Supplements: As dictated by the protocol: one sample dilution with 2.5 mg protein per ml and a second sample dilution with 0.25 mg protein per ml.
   Dilutions for Human Breast Milk: As dictated by the protocol: one sample dilution with 2.5 mg protein per ml and a second sample dilution with 0.25 mg protein per ml.
2. Add 25 μL of the sodium meta-periodate solution to each well.
3. Mix plate for 30 seconds in a microplate shaker.
4. Cover and incubate plate at room temperature for 10 minutes.
5. Add 150 μL of the Glycoprotein Detection Reagent to each well.
6. Mix plate for 30 seconds in a microplate shaker.
7. Cover and incubate plate at room temperature for 1 hour.
8. Mix plate for 5 seconds in a microplate shaker. Measure absorbance at 550 nm in a microplate reader and plot a standard curve.

Results:

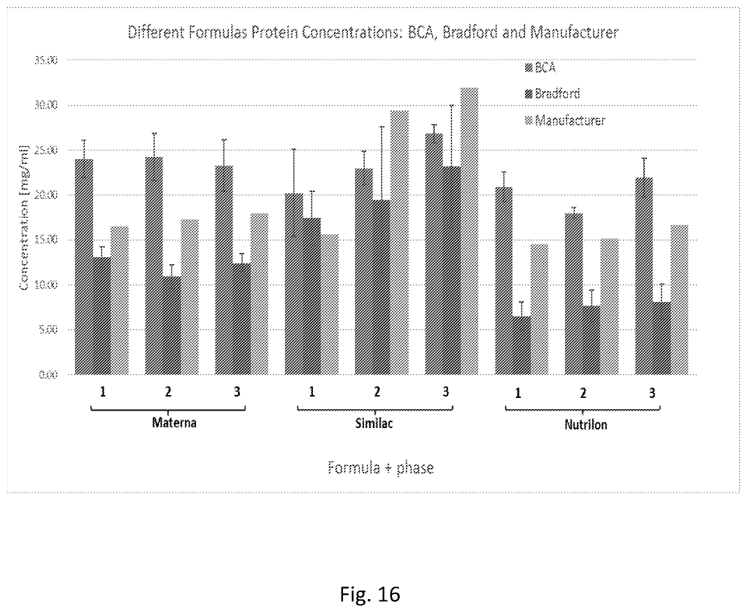

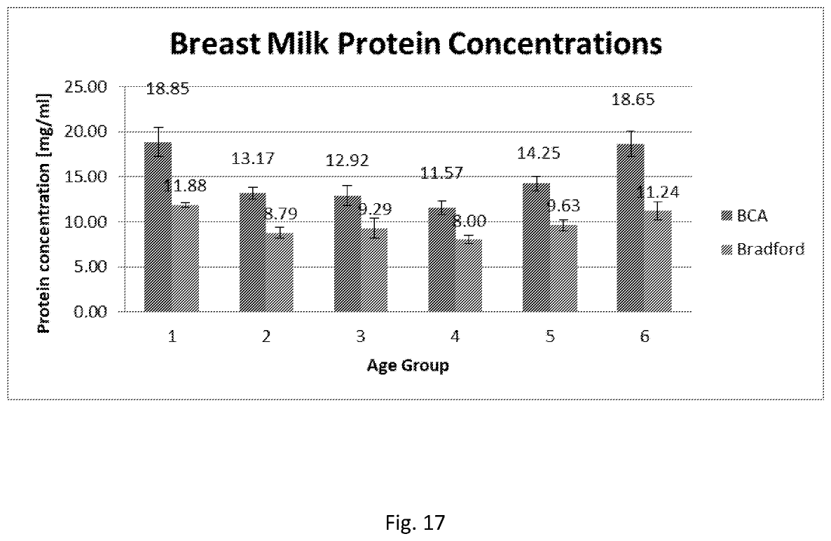

We noticed that the results of the Bradford test give protein values that are generally lower than the manufacturer's values, whereas the results of the BCA test give values that are higher than the manufacturer's (see FIG. 16).

In Both the Bradford and the BCA tests, a unique trend is observed: the highest protein concentration is in age group 1 (BCA: 18.85±1.63 mg/ml, Bradford: 11.88±0.29 mg/ml), with a decline until stage 4 (BCA: 11.57±0.74 mg/ml, Bradford: 8.00±1.03 mg/ml, then again incline until stage 6 (BCA: 18.65±1.42 mg/ml, Bradford: 11.24±1.02 mg/ml) (See FIG. 17). However, it is not clear whether the highest concentration is at stage 1 or 6, as well as whether the lowest concentration is at stage 2, 3, 4 or 5. with some mothers this trend was not clear. Similar to the tests of the formulas, the Bradford assay always showed lower concentrations than the BCA assay.

Discussion

Proteins

It is clear from FIG. 16 that both BCA and Bradford assays don't give identical results to the manufacturer's values, nor identical to one another. In addition, there is no single mathematical factor that can be calculated for the conversion the assays' values to the manufacturer's, not to mention that two results showed higher values for the Bradford assay compared to the manufacturer's, in contrast to the general opposite behavior. Nevertheless, with further repeats and calibration such factor may be found, since both methods are credible for protein quantification. It's surprising that the Bradford method was allegedly inaccurate, since the literature claims that Bradford method is the most accurate for total protein quantification in milk, regardless of the presence of lipids. In order to better determine the true accuracy of this method, we can use standards of Albumin and Casein. It is here to mention that the milk samples were frozen at −80° C. and then defrosted two times, a fact that may have an impact on the protein concentration in each test.

In FIG. 17 it can be observed that the hypothesis regarding the protein concentration was partially approved: there is a decline of the protein concentration as the baby grows older. The colostrum and the transitional milk are known to have the highest concentration of proteins, for they include a concentrated dose of immune factors that are necessary for the baby's immune system. However, there is an increase in the protein concentration in the fifth and sixth age groups. It is reported in the literature that while there is a decrease in the protein concentration from 4-6 months to 12-20 months in milk of full-lactating women (Volume>500 ml/day), when the volume of milk is unknown (only known that >=4 feedings/day) there is an increase in the protein concentration. It is also reported that protein concentration increases as volume of lactated milk decreases. Older babies start to wean from breast milk and feed on other food, a fact that can explain the above results.

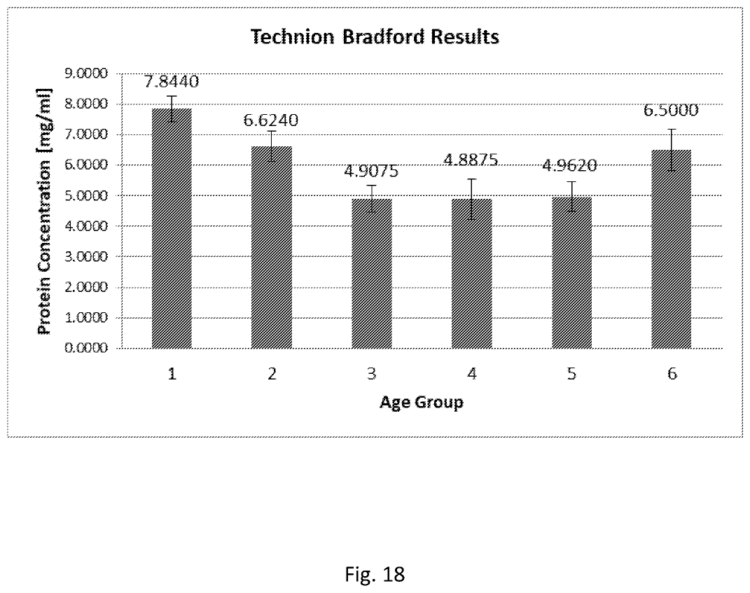

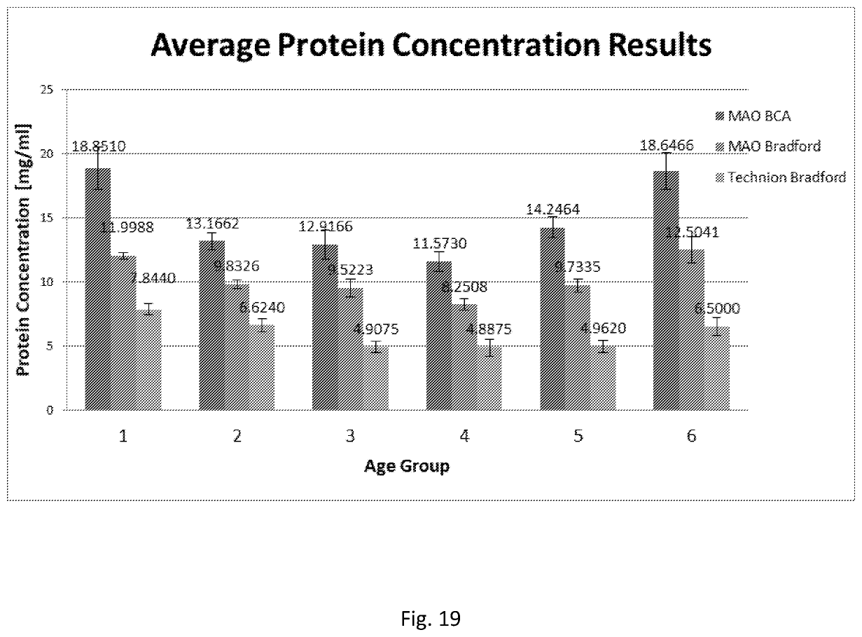

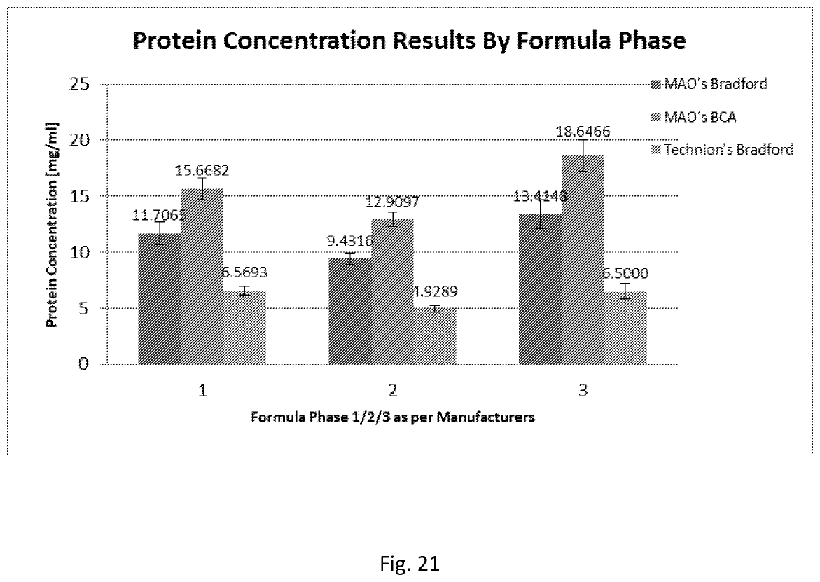

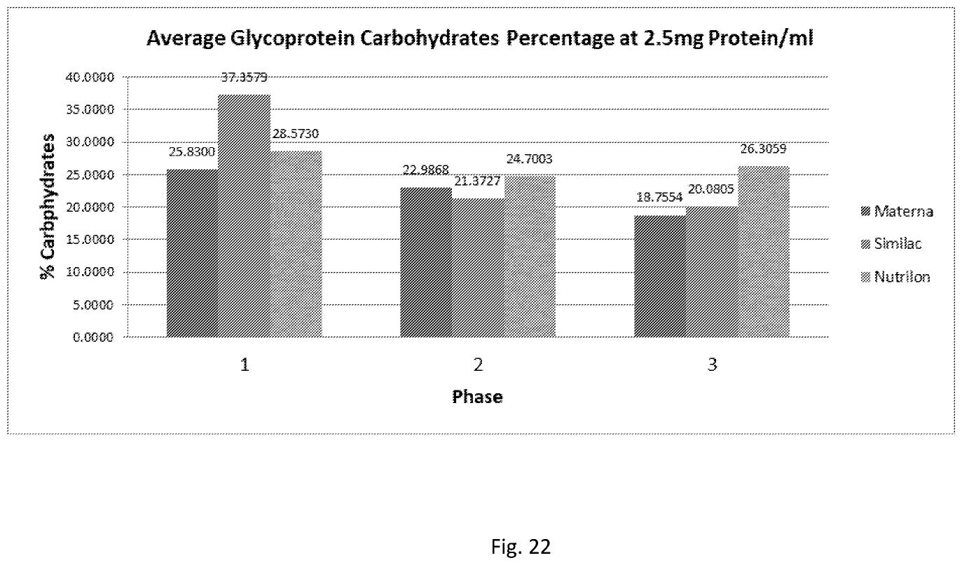

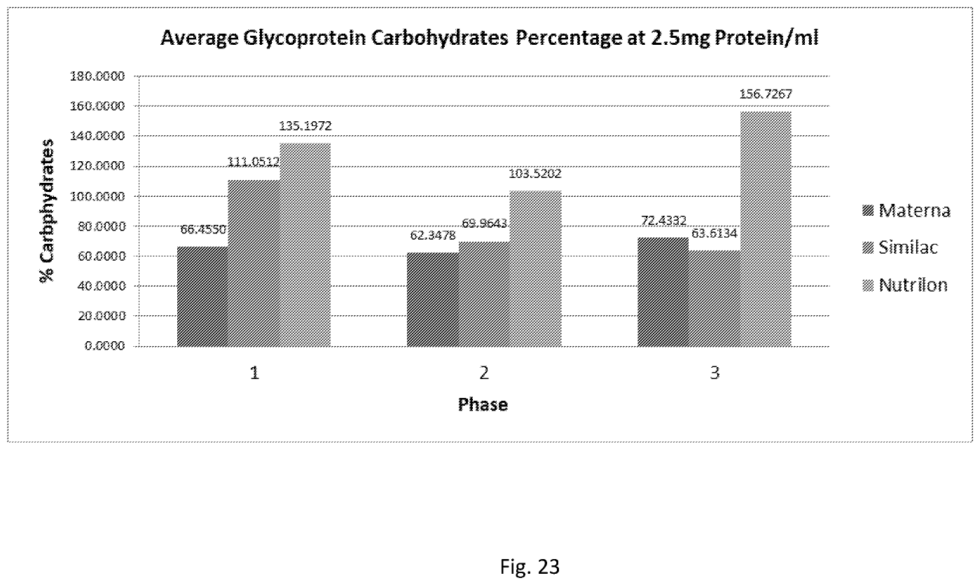

As part of a second experiment taking place at the Technion Institute's Protein Lab, a set of five breast milk samples from each age group were tested for protein concentration using the Bradford method. Some of these samples were from the same mother of the samples in this experiment, and some were from different mothers. It is possible to observe in FIG. 18 the same protein concentration trend as in FIG. 17. The values obtained by the Technion group were lower by comparison than those obtained by our results (referred to herein as “MAO”) see FIG. 19), but the decrease in protein concentration observed by the MAO team from age group 1 to age group 4, and subsequent increase in protein concentration in the latter age groups, can also be seen in the Technion's findings. This similar trend can be observed even if the lactation age is divided by the usual commercial division of phases—phase 1 is for infants aged 0-6 months, phase 2, for infants aged 6-12 months and phase 3 for infants over 12 months old. In FIG. 21, a similar decrease between the first months of lactation up to the twelfth month of lactation can be observed, although the definition for infants aged 9 and above is lost, therefore the increase in protein is only observed at phase 3 and not at group 5 as before.

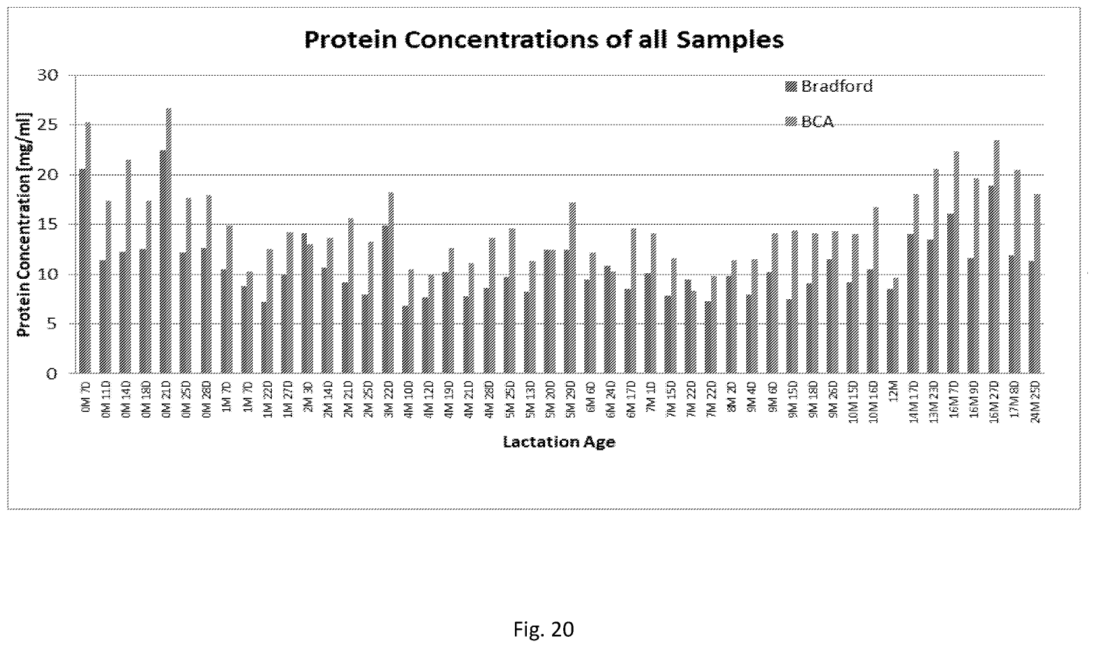

Although between each age group there is a noticeable difference in the group's average protein concentration, within each group individually there is no significant correlation between the age of the infant and the protein concentration of the breastmilk sample (FIG. 20). This means that there is variance in the protein concentration between mothers, which can also be observed in the standard errors of the results. This could be a result of various factors: mother's nutrition, mother's age and history of pregnancy, physical and health status of the mother or the infant, time in the day of the collection of the samples, etc.

Carbohydrates

The test used to determine the glycoprotein carbohydrates concentration within each breast milk and formula sample was the Thermo Scientific Glycoprotein Carbohydrate Estimation Kit, which depends on a set protein concentration for the creation of a standard curve. The two protein concentrations required by the kit were 0.25 mg of protein per 1 ml of liquid or 2.5 mg of protein per 1 ml of liquid. However both the formulas and the breastmilk samples used in this experiment had a higher glycoprotein concentration than the kit could detect. At 2.5 mg protein per ml, the samples were overexposed and at 0.25 mg protein per ml, the OD results were far outside the range of the standard curve.

Although the numbers cannot be claimed as accurate, there is no observable trend in FIGS. 22 and 23 among the three infant food formulas. Each formula follows its own pattern of increase and decrease in carbohydrates percentage and glycoprotein concentration, and that trend is for the most part consistent between the results from the 2.5 mg protein per ml and the 0.25 mg protein per ml samples (with the exception of Materna, where a steady decrease seen in FIG. 22 cannot be observed in FIG. 23). On the formula's labels, Materna boasted of a steady increase in carbohydrates content from phase 1 to phase 2 and finally to phase 3, while Similac showed a more steady concentration through the three phases and Nutrilon showed an increase between phases 1 and 2 and a decrease between phases 2 and 3. Only Nutrilon's results using this kit somewhat matched the trend displayed on the products' labels. Since this kit tests for glycoprotein carbohydrates percentage, and the products' label do not make a distinction between carbohydrates that are found in glycoproteins and free carbohydrates, it was not expected that the results would match.

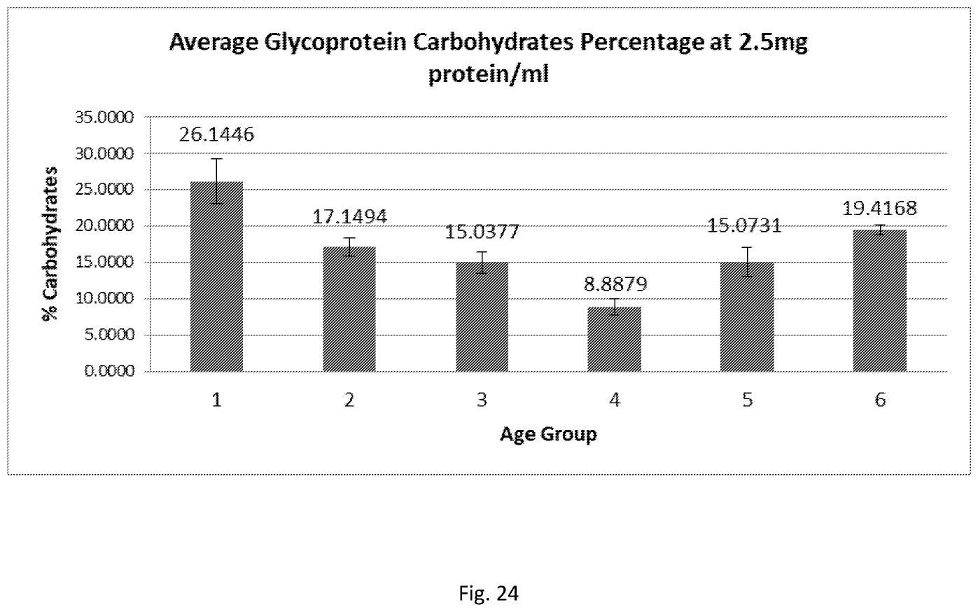

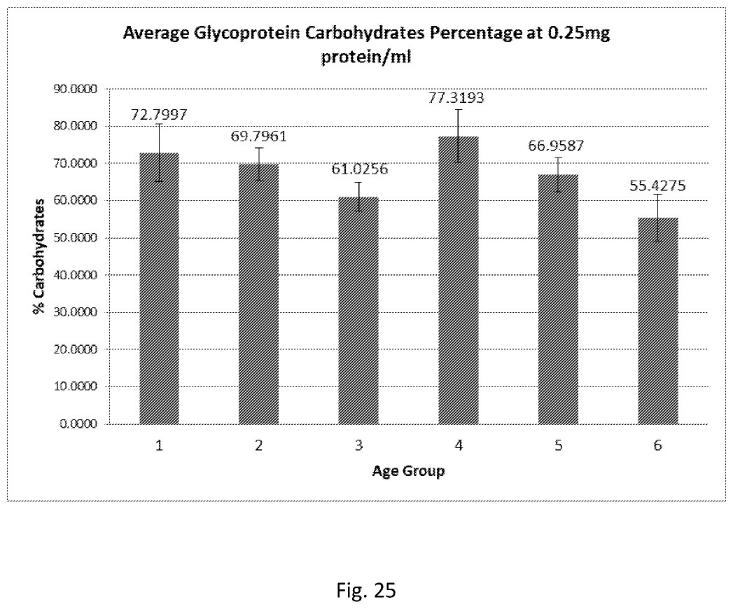

For the breastmilk samples, an interesting trend can be seen in FIG. 24. Although these results were obtained using samples that were overexposed, a similar trend as that of the protein concentrations can be observed in the different age groups' glycoprotein carbohydrates percentage. Age group 1 shows the highest percentage of carbohydrates, with a steady decrease until age group 4. Age group 5 shows an increase in the carbohydrates percentage, which carries on to age group 6. These results were expected, since the kit used in this experiment ultimately tests for glycoproteins, which are part of the total protein concentration estimated using BCA and Bradford. The results further suggest that there is a higher concentration of glycoproteins in the first month of lactation (group 1) than after a year of lactation (group 6), even though FIG. 17 shows that the total protein concentrations for these two groups is similar. FIG. 25 does not corroborate the trend found in FIG. 24.

According to some demonstrative embodiments, the device of the present invention may allow for the analysis and/or monitoring of one or more components in the breastmilk, and optionally provide an indication regarding the nutritional and/or immunological needs of a breast fed infant which is at least partially fed by the breastmilk.

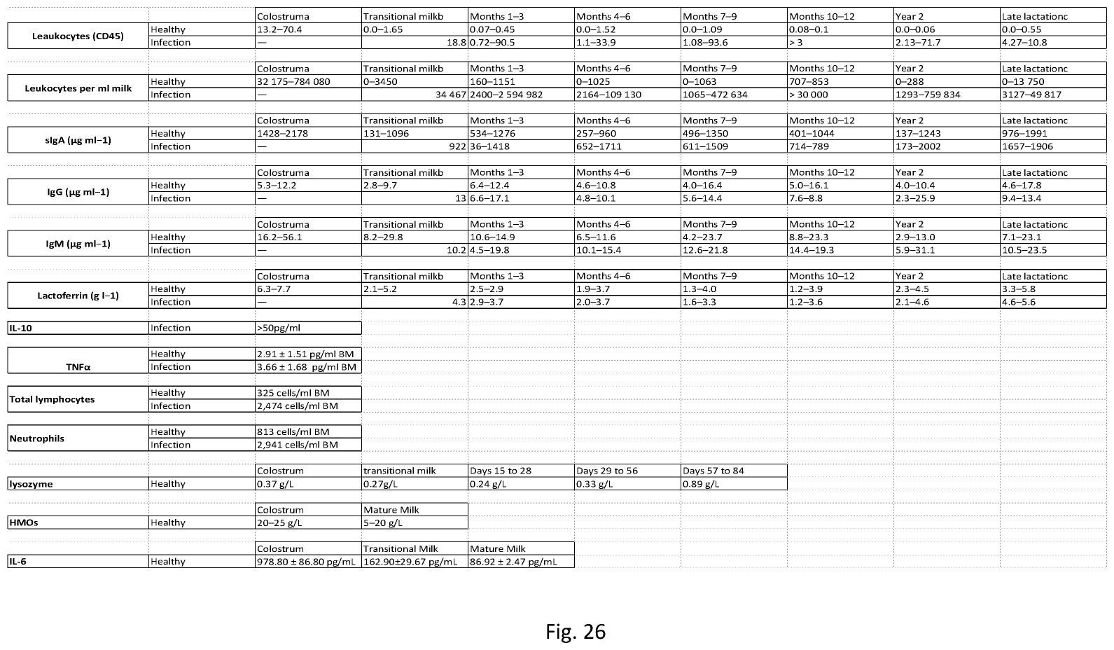

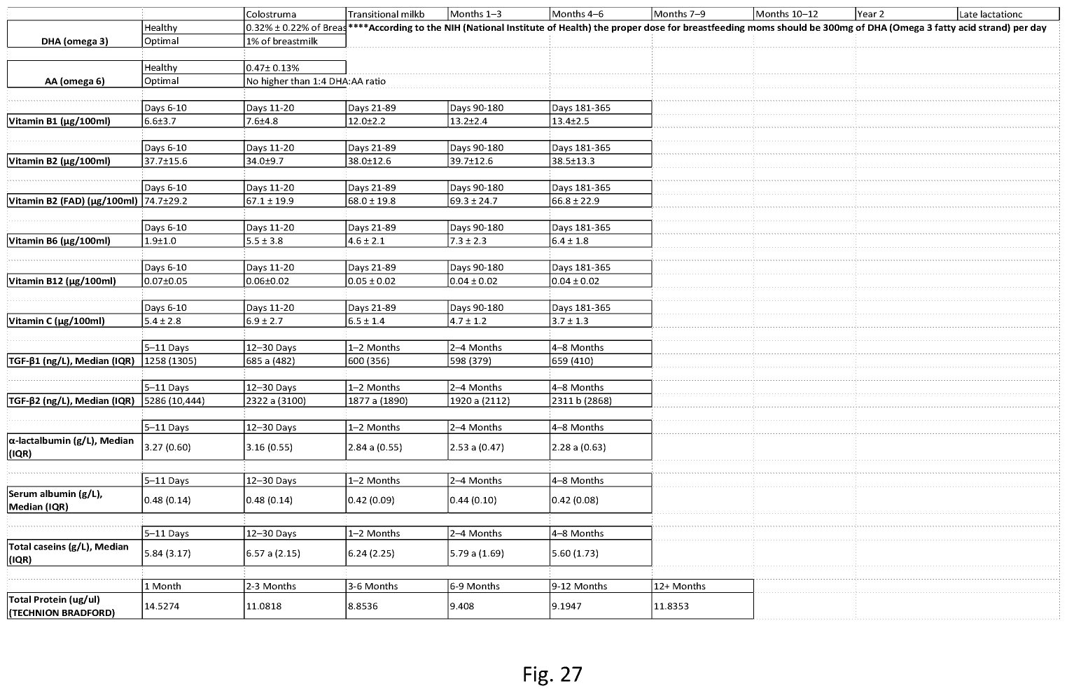

According to some embodiments, the components in the breastmilk may include one or more of the candidates appearing in the tables depicted in FIGS. 26 and 27.

According to some embodiments, the device may be pre-configured with normal ranges, e.g., concentrations, for each of the components, and optionally the device may also be pre-configured with non-normal ranges od the components, e.g., indicating and infection.

According to some embodiments, upon analysis of one or more components the device may indicate whether the one or more components are within or outside the normal range, and optionally provide a recommendation for the user of the device.

### Example 12

For concentration results, which vary over time, the best testing method is using a legend with a color gradient. The stick will not give a numerical result which can be directly compared to that expected concentration, but rather a hue which directly correlates to the amount of protein found in the sample (the stronger the hue, the more protein in the sample). The user can compare the clue stain which will appear on the stick directly with the following legend, and extrapolate the results of the test from there.

TABLE 21
Total Protein Concentration using a colorimetric result

| | 0 to 1 | 1 to 3 | 3 to 12 | 12 Months |
|---|---|---|---|---|
| Age | Months | Months | Months | Over |
| Protein | 14.50 ± 0.2 | 11.1 ± 0.05 | 9.15 ± 0.1 | 11.85 ± 0.2 |
| Concentration | | | | |
| (mg/ml) | | | | |

1. The user will dip the Total Proteins Concentration stick (sampling element) in the breastmilk to the marked line
2. The user will wait the indicated amount of time for the reaction to occur, preferably less than 10 minutes, more preferably, less than 5 minutes.
3. The user will compare the reaction to the legend.
4. The user will read the instructions on the legend: if the color on the stick matches or is above the total protein concentration for the infant's age group, the user does not have to make any changes, e.g., because the protein concentration in the breastmilk corresponds or is higher than the desired concentration for the infant in accordance with its age. If the color on the stick matches a protein concentration which is lower than the infant's age group, it will be recommended for the mother (whose breastmilk was tested) to increase her protein intake throughout the day.

For example, for a mother of a 2.5 months old infant, the total protein concentration average is expected to be around 11.1±0.05 (mg/ml). If the total protein concentration in the tested milk is 9.00 mg/ml, the colorimetric result on the stick will match a concentration outside the range of the infant's age group. In such a case, the mother is recommended to increase her protein intake through her daily nutrition or through supplements.

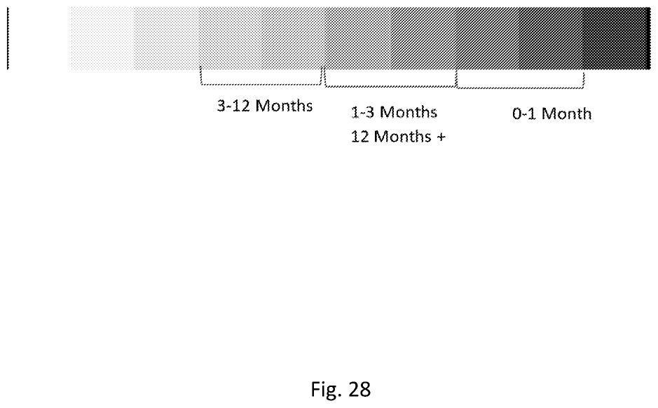

Reference is made to FIG. 28 which shows an exemplary legend in accordance with some demonstrative embodiments, according to which a user of a sampling element of the present invention may get an indication regarding the levels of protein concentrations in the breastmilk.

According to some embodiments, a user of the sampling element will compare the results indicated upon the sampling element to the legend's colors, and may verify a correspondence with the expected protein concentration.

FIG. 28 shows the legend's colors which correspond to the protein concentration according to the expected age of the infant. If the tested sampling element shows colors which are brighter than those of the expected age of the infant, this may mean the mother's breastmilk contains protein concentration which is lower than the expected and/or desired for her infant at that specific age.

The expected protein concentrations represented by the legend shown in FIG. 28 are visual representations of the information of table 21 and are based on known data derived from the literature.

### Example 13

TABLE 22
Total Protein Concentration in different formulas

| | Formula 1 (Materna) | | | Formula 2 (Nutrilon) | | | Formula 3 (Similac) | | |
|---|---|---|---|---|---|---|---|---|---|
| | Phase 1 | Phase 2 | Phase 3 | Phase 1 | Phase 2 | Phase 3 | Phase 1 | Phase 2 | Phase 3 |
| Protein | 14.6 | 15.8 | 16.6 | 13 | 14 | 15 | 14 | 28 | 28 |
| mg/ml | | | | | | | | | |

As shown in table 22, three different infant formulas were examined for their protein concentration at each phase.

TABLE 23
Average Total Protein Concentration according to
the age of an infant

| | 0-6 Months | 6-12 Months | 12 Months Over |
|---|---|---|---|
| Age by Phases | (Phase 1) | (Phase 2) | (Phase 3) |
| Protein | 11.4877 ± 0.1 | 9.3013 ± 0.1 | 11.8353.2 |
| Concentration | | | |
| (mg/ml) | | | |

1. The user will dip the sampling element in the breastmilk up to a predetermined marked line
2. The user will wait the indicated amount of time for the reaction to occur, preferably less than 10 minutes, more preferably, less than 5 minutes.
3. The user will compare the results appearing on the sampling element to the legend's indicators
4. The user will read the instructions on the legend: The color on the sampling element will tell the user which baby supplement phase best suits the infant, according to the total protein concentration in the milk.

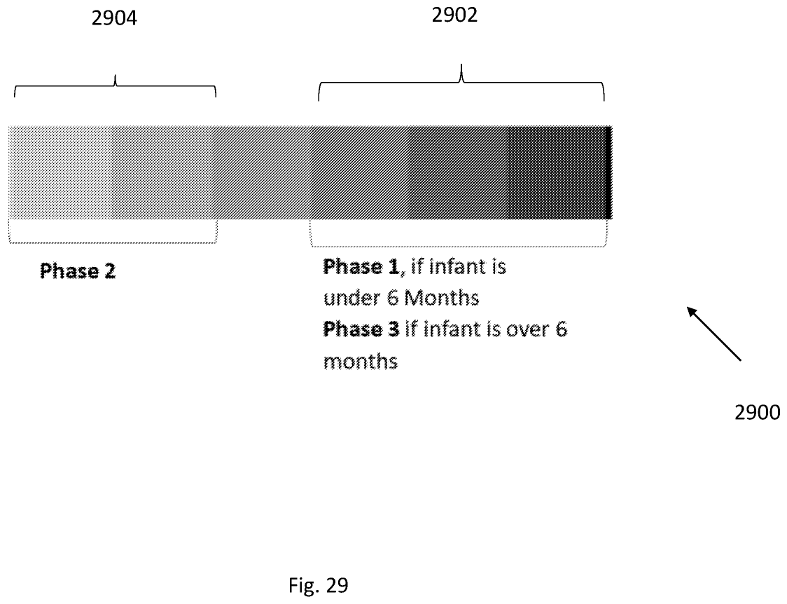

Reference is made for example to FIG. 29 which demonstrates an exemplary legend 2900, to which a sampling element result may be compared to. According to FIG. 29, if the sampling element shows a color which corresponds to the colors 2904, this may indicate that the infant should be consuming a formula phase 2, which corresponds to the protein concentration in the breastmilk of his mother. However, if the sampling element shows a color which corresponds to the colors 2902, this may indicate that the infant should be consuming a formula phase 1 (if the infant is under the age of 6 months), or formula phase 3 (if the infant is over the age of 6 months).

### Example 14

TABLE 24
Average Vitamin B Concentration in breastmilk according
to the age of a breastfed infant

| | Days | Days | Days | Days | Days |
|---|---|---|---|---|---|
| Vitamins B: | 6-10 | 11-20 | 21-89 | 90-180 | 181-365 |
| Vitamin B1 | 6.6 ± 3.7 | 7.6 ± 4.8 | 12.0 ± 2.2 | 13.2 ± 2.4 | 13.4 ± 2.5 |
| (μg/100 ml) | | | | | |
| Vitamin B2 | 37.7 ± 15.6 | 34.0 ± 9.7 | 38.0 ± 12.6 | 39.7 ± 12.6 | 38.5 ± 13.3 |
| (μg/100 ml) | | | | | |
| Vitamin B2 | 74.7 ± 29.2 | 67.1 ± 19.9 | 68.0 ± 19.8 | 69.3 ± 24.7 | 66.8 ± 22.9 |
| (FAD) | | | | | |
| (μg/100 ml) | | | | | |
| Vitamin B6 | 1.9 ± 1.0 | 5.5 ± 3.8 | 4.6 ± 2.1 | 7.3 ± 2.3 | 6.4 ± 1.8 |
| (μg/100 ml) | | | | | |
| Vitamin B12 | 0.07 ± 0.05 | 0.06 ± 0.02 | 0.05 ± 0.02 | 0.04 ± 0.02 | 0.04 ± 0.02 |
| (μg/100 ml) | | | | | |

The Vitamin B (B1, B2, B6 or B12) in question will be referred to as Vitamin Bx in this example
   1. The user will dip the Vitamin Bx Concentration stick (sampling element) in the breastmilk up to a predetermined marked line
   2. The user will wait the indicated amount of time for the reaction to occur, preferably less than 10 minutes, more preferably, less than 5 minutes.
   3. The user will compare the reaction to the legend
   4. The user will read the instructions on the legend: if the color on the stick matches or is above the concentration of the Vitamin Bx for the infant's age group, the user does not have to make any changes. If the color on the stick matches a Vitamin Bx concentration which is lower than the infant's age group, it will be recommended for the mother (whose breastmilk was tested) to increase her Vitamin Bx intake throughout the day

According to some demonstrative embodiments, the invention may include a pack of Vitamin B sampling elements which may include test sticks, a legend and a list of foods recommended to increase the Vitamin Bx levels in the breastmilk

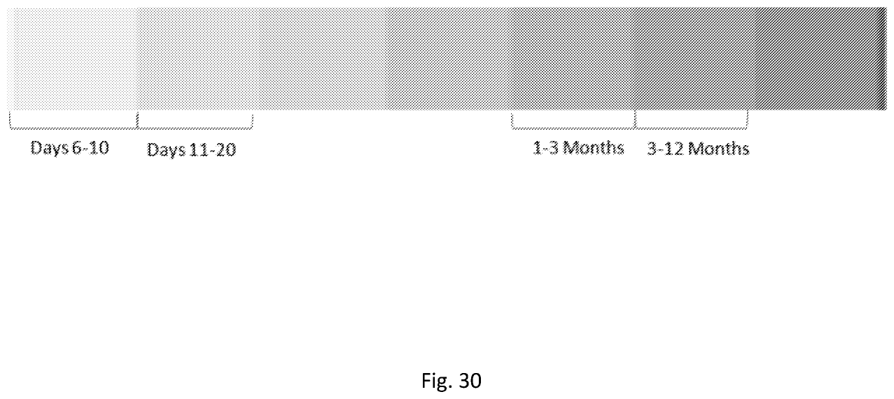

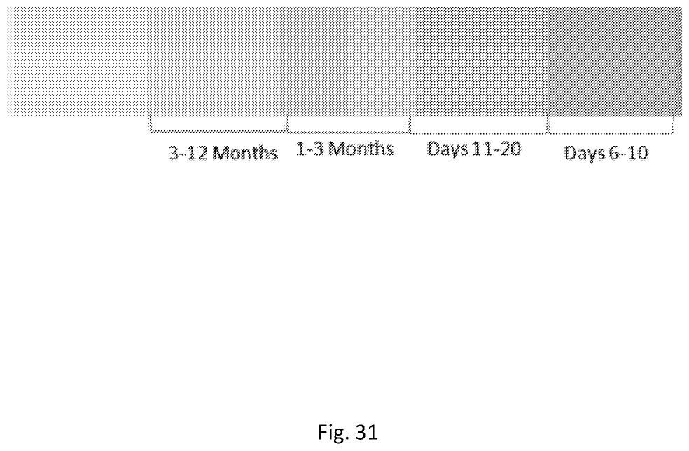

Reference is made to FIG. 30, which illustrates an exemplary legend of desired concentration of Vitamin B1 in the breastmilk of a mother feeding an infant in correlation to the age of the infant.

According to some embodiments, and as referred to in FIG. 30, if the Vitamin B1 concentration in the breastmilk for an infant at the age of 2 months is about 8 ng/100 ml, the color indicator on the sampling element will not match the respective color on the legend for infants between 1-3 months of age, which will tell the user that the vitamin B1 concentration in the tested breastmilk is lower than the average, e.g., the desired concentration for an infant aged 2 months, and the mother will be recommended to ingest more Vitamin B1 in her daily nutritional intake.

Reference is made to FIG. 31, which illustrates an exemplary legend of desired concentration of Vitamin B12 in the breastmilk of a mother feeding an infant in correlation to the age of the infant.

According to some embodiments, and as referred to in FIG. 31, if the Vitamin B12 concentration in the breastmilk for an infant at the age of 2 months is about 0.03 μg/100 ml, the color indicator on the sampling element will not match the respective color on the legend for infants between 1-3 months of age, which will tell the user that the vitamin B12 concentration in the tested breastmilk is lower than the average, e.g., the desired concentration for an infant aged 2 months, and the breastfeeding mother will be recommended to ingest more Vitamin B12 in her daily nutritional intake.

### Example 15

Long Chain—Polyunsaturated Fatty Acids (LC-PUFA): Using a panel and smartphone application to decipher the results of the sampling element

This sampling element will test two LC-PUFA molecules, Omega 3 and Omega 6, wherein the recommended ratio of Omega 3 to Omega 6 should be 1:4 at most, with the optimal ratio being 1:1
1. The user will dip the LC-PUFA stick (sampling element) in the breastmilk up to a predetermined marked line
2. The user will wait the indicated amount of time for the reaction to occur, preferably less than 10 minutes, more preferably, less than 5 minutes
3. The user will place the stick on the accompanying color panel at the indicated location
4. The user will use her smartphone, open a dedicated app and scan the color panel with the stick using the application.
5. The application will calculate the ratio of Omega 3 to Omega 6 and tell the user the results and recommendations. If the ratio is above a 1:4 ratio, the app will recommend the mother whose breastmilk was tested to increase the omega 3 intake in her diet by, for example, consuming more fatty fish.

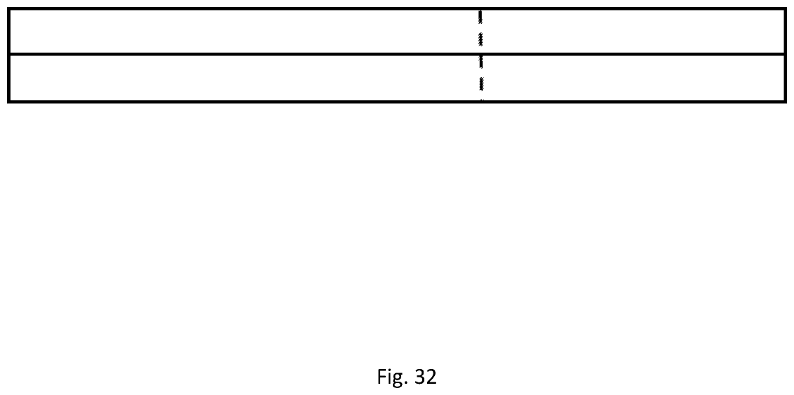

FIG. 32 is an illustration of a sampling element for LC-PUFA with a predetermined marked line, to indicate to the user of the sampling element the depth up to which the user should dip the element into the tested breastmilk.

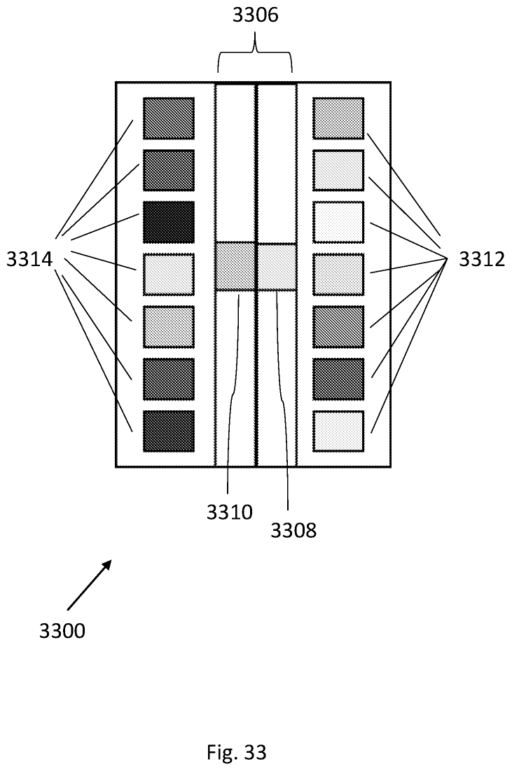

FIG. 33 is an illustration of an exemplary control panel 3300, wherein sampling element 3306 is inserted into panel 3300, and the results indicated upon element 3306 can be compared to the reference results indicated upon panel 3300.

According to some embodiments, sampling element 3306 may be dipped into or contacted by a suitable amount of breast milk, e.g., 5 ml, and a reaction may occur to indicate the concentration of Omega 3 and/or Omega 6 in the breastmilk. According to some embodiments, this indication of the results may be presented by indicator 3310 for Omega 3 and indicator 3308 for Omega 6.

According to some embodiments, panel 3300 may have reference indicators, e.g., indicators 3314 for reference of various concentrations of Omega 3, and indicators 3312 for reference of various concentrations of Omega 6.

According to some embodiments, after insertion of sampling element 3306 into panel 3300 the result indicators 3310 and 3308 may be compared to reference indicators 3314 and 3312, respectively.

According to some embodiments, this comparison may be done by a user using naked eye or alternatively, using any suitable method that may allow an easy comparison of the result indicators 3310 and 3308 to reference indicators 3314 and 3312, including, for example, a smart phone application which may enable a user to take a picture of the sampling element 3306 within panel 3300 and provide an immediate feedback regarding the concentration of Omega 3 and/or Omega 6 in the tested breastmilk.

### Example 16

TABLE 25
Average Leukocytes count in a healthy or infected breastmilk

| | | | Transitional | Months | Months | Months | Months | | Late |
|---|---|---|---|---|---|---|---|---|---|
| | | Colostrum | milk | 1-3 | 4-6 | 7-9 | 10-12 | Year 2 | lactation |
| Leukocytes | Healthy | 32,175- | 0-3,450 | 160-1,151 | 0-1,025 | 0-1,063 | 707-853 | 0-288 | 0-13,750 |
| (CD 45) | | 784,080 | | | | | | | |
| per ml | Infection | — | 34,467 | 2,400- | 2,164- | 1,065- | >30,000 | 1,293- | 3,127- |
| milk | | | | 2,594,982 | 109,130 | 472,634 | | 759,834 | 49,817 |

According to this example, there is provided a sampling element for detecting the presence and/or amount of Leukocytes (CD 45) in the tested breastmilk.

According to this example the sampling element may include a control indicator and a positive or negative indicator (also referred to as Yes/No indicator), however, it is to be understood that the sampling element may also have a range indicator, in addition or instead of the positive or negative indicator.
1. The user will dip the sampling element in the breastmilk up to a predetermined marked line
2. The user will wait the indicated amount of time for the reaction to occur, preferably less than 10 minutes, more preferably, less than 5 minutes
3. The user will interpret the results based on the guide on the sticks pack
4. A control mark must always appear on the stick. If the control mark does not appear, the reaction did not occur properly.

If the infant or mother is developing an infection, CD-45 levels in the tested breastmilk would be elevated. For example, for an infant 2 months old that is beginning to develop an infection, CD-45 levels would be over 4000 particles per ml of milk. In such a case, the sampling element would have a positive control indicator, indicating that the sampling element is working properly, and a positive indication in the Yes/No indicator.

### Example 17

According to this example, there is provided a sampling element (Lactoferrin stick) for detecting the presence and/or amount of Lactoferrin in the tested breastmilk.

According to this example the sampling element may include a control indicator and a positive or negative indicator (also referred to as Yes/No indicator), however, it is to be understood that the sampling element may also have a range indicator, in addition or instead of the positive or negative indicator.

TABLE 25
Average Lactoferrin concentration in a healthy or infected breastmilk

| | | | Transitional | Months | Months | Months | Months | |
|---|---|---|---|---|---|---|---|---|
| | | Colostrum | milk | 1-3 | 4-6 | 7-9 | 10-12 | Year+ |
| Lactoferrin | Healthy | 6.3-7.7 | 2.1-5.2 | 2.5-2.9 | 1.9-3.7 | 1.3-4.0 | 1.2-3.9 | 2.3- |
| (g/l) | | | | | | | | 4.5 |
| | Infection | — | 4.3 | 2.9-3.7 | 2.0-3.7 | 1.6-3.3 | 1.2-3.6 | 2.1- |
| | | | | | | | | 4.6 |

1. The user will dip the Lactoferrin stick in the breastmilk to the marked line
2. The user will wait the indicated amount of time for the reaction to occur
3. The user will interpret the results based on the guide on the sticks pack

If the infant or mother is developing an infection, Lactoferrin levels in the tested breastmilk should be elevated. For example, for an infant 2 months that is beginning to develop an infection Lactoferrin levels should be over 3 g/L. In such a case, the sampling element would have a positive control indicator, indicating that the sampling element is working properly, and a positive indication in the Yes/No indicator.

### Example 18

TABLE 27
Average sIgA concentration in a healthy or infected breastmilk

| | | | Transitional | Months | Months | Months | Months | 1 |
|---|---|---|---|---|---|---|---|---|
| | | Colostrum | milk | 1-3 | 4-6 | 7-9 | 10-12 | Year+ |
| sIgA | Healthy | 1428- | 131-1096 | 534- | 257- | 496- | 401- | 137- |
| (μg/ml) | | 2178 | | 1276 | 960 | 1350 | 1044 | 1243 |
| | Infection | — | 922 | 36- | 652- | 611- | 714- | 173- |
| | | | | 1418 | 1711 | 1509 | 789 | 2002 |

According to this example, there is provided a sampling element for detecting the presence and/or amount of Immunoglobulins, e.g., sIgA in the tested breastmilk.

According to this example the sampling element may include a control indicator and a positive or negative indicator (also referred to as Yes/No indicator), however, it is to be understood that the sampling element may also have a range indicator, in addition or instead of the positive or negative indicator, and a control indicator to indicate whether the element is working properly.
1. The user will dip the sampling element in the breastmilk to the marked line
2. The user will wait the indicated amount of time for the reaction to occur
3. The user will interpret the results based on the guide on the sticks pack
4. A control mark must always appear on the stick. If the control mark does not appear, the reaction did not occur properly.

If the infant or mother is developing an infection, sIgA levels in the tested breastmilk would be elevated. For example, for an infant 2 months that is beginning to develop an infection sIgA levels should be over 1300 μg/ml. In such a case, the sampling element would have a positive control indicator, indicating that the sampling element is working properly, and a positive indication in the Yes/No indicator.

According to some embodiments, there is provided a sampling element, also referred to herein as an immunological stick, which may contain more than one immunological test. According to these embodiments, the immunological stick may include a test for Leukocytes, Lactoferrin and an Antibody test.

According to these embodiments, the immunological stick may provide a more comprehensive indication about the infant's and/or mother's health condition, in comparison to a single test.

According to some demonstrative embodiments, there is provided herein a devise to enable the analysis of the composition of breastmilk, for example, for assessment of the nutritional and/or immunological condition of a breast fed infant which is fed by the breastmilk.

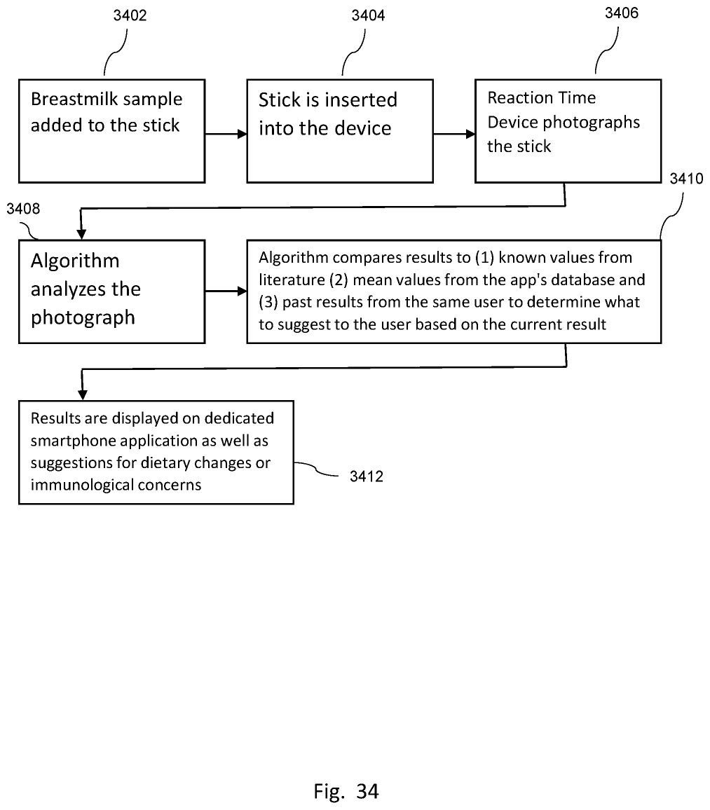

Reference is now made to FIG. 34 which illustrates a flow chart depicting the action of the device.

As shown in FIG. 34, on block 3402 a breast milk sample may be added to a sampling element referred to in the drawing as a stick.

Block 3404 discloses that the stick may be inserted in the device.

Block 3406 described that a reaction may occur within the sampling element, e.g., to identify the existence and/or concentration or amount of one or more target molecule present in the sampled breastmilk, wherein the results of the reaction are demonstrated externally on the stick.

Block 3406 also details that the device may photograph the stick, e.g., the device may include a camera that may photograph the results of the reaction as externally depicted, see for example, with regard to FIG. 35.

Block 3408 details that the photographs taken by the device may be analyzed in accordance with a pre-defined algorithm, as detailed herein.

Block 3410 details that the pre-defined algorithm may include pre-defined parameters and/or values that may be selected from the group including (1) known values of target molecules, e.g., from literature (2) mean values of target molecules, taken for example, from the device's database and (3) past results of target molecules analyzed by the device from the same user.

According to some embodiments, the results analyzed as per block 3408 may be compared with the predefined parameters and/or values, for example, to determine and/or present a recommendation to the user of the device.

According to some embodiments, block 3412 details that the results may be displayed on dedicated application, e.g., a smartphone application, as well as suggestions for dietary changes or immunological concerns.

### Example 19

Provided herein is an example of a calculation of the concentration of a specific target in a sample using standards and the ELISA method. In this example we are looking at IL-10, that standard concentrations are as described. After reading the results with an ELISA reader, the OD for each standard is as described.

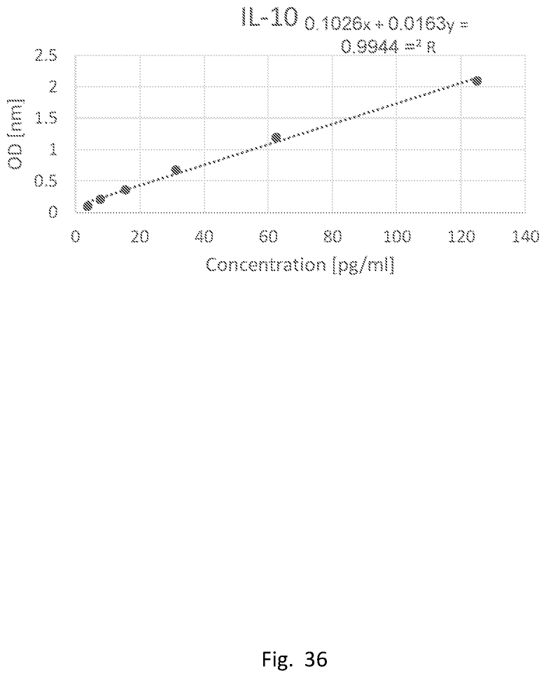

The results of this example are depicted in the graph of FIG. 36, from which this equation OD=0.0613 [conc]+0.1026 is deducted.

Another important point that comes with the graph is the R² value, which is a score between 0-1 that denotes how accurately close the linear regression reflects reality. The closer the R² value is to 1, the more accurate the regression is considered. Usually, the cutoff number is 0.95 (underneath which the test must be repeated). We are currently using 0.98 as our cutoff, although this may change.

After extracting the linear equation from the standards and OD, it is not possible to extrapolate the concentration of IL-10 in any sample, as long as the OD for that sample falls within the range of ODs for the standards. The reason we put this stipulation (not to surpass the range of standard OD) is that we can only know for sure the statistical accuracy of the linear equation within the boundaries of the samples used to create the equation. Outside those boundaries, results acquired using the linear equation can not be evaluated for accuracy and therefore must be annulled.

Target: IL-10

Standards: 3.9, 7.8, 15.6, 31.3, 62.5, 125 pg/ml

TABLE 27

| Concentration | OD |
|---|---|
| Blank | 0 |
| 3.9 | 0.098833 |
| 7.8 | 0.210633 |
| 15.6 | 0.35895 |
| 31.3 | 0.6756 |
| 62.5 | 1.192933 |
| 125 | 2.094633 |

For a sample with an OD of 0.5178 we can use the linear equation as follows:

OD=0.0613[conc]+0.1026

[conc]=(OD˜0.1026)/0.0613

[conc]=25.47 pg/ml

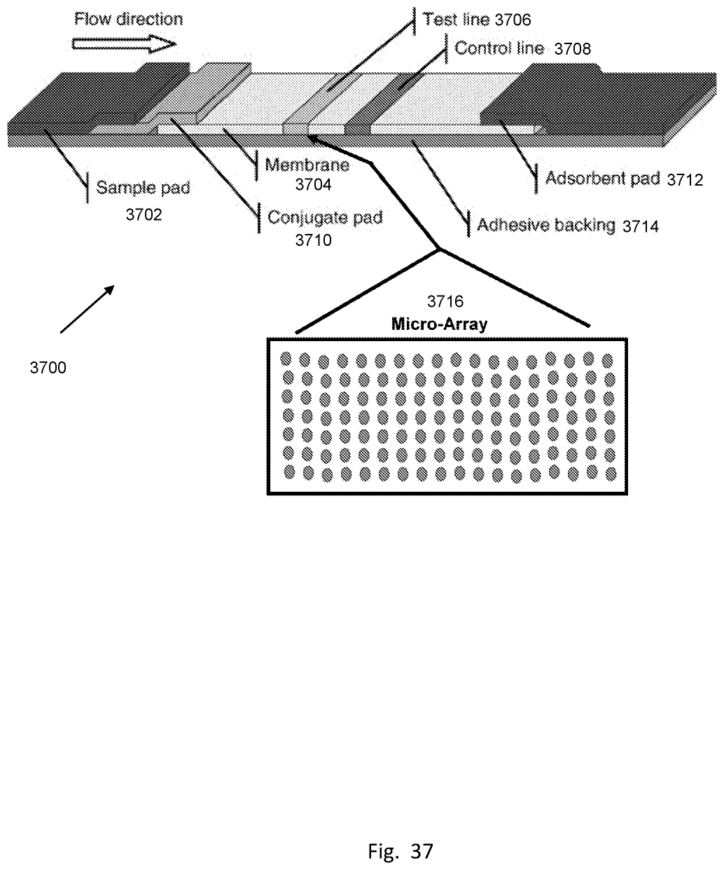

Reference is now made to FIG. 37 which depicts a sampling element 3700 is accordance with some demonstrative embodiments.

Sampling element 3700 may include a lateral Flow Stick with a microarray of molecules, e.g., reagents including antibodies, proteins, vitamins and the like, e.g., located at test line 3706, also referred to as a reaction Zone.

According to some embodiments, the lateral flow of sampling element 3700 is based on the flow of the liquid from one side to the other. There are different key components in the lateral flow:

Sample pad 3702: an area onto which a user places a sample and that can absorb the liquid from the sample quickly.

Conjugate pad 3710: According to some embodiments, pad 3710 may be made of glass fibers or other suitable materials that can potentially hold antibodies/proteins etc., but in a weak manner that will release them into the liquid once it reaches that area/upon wetting.

There usually is a secondary spacer made of glass fibers or alike, which will serve as a buffer zone between the Conjugate pad and the Membrane and will allow for better flow into the Membrane.

Membrane 3704, According to some embodiments, membrane 3704 may be made of nitrocellulose or polymer fibers arranged in an array that may include pores and spaces which filter the desired target molecules from undesired molecules (also known as unnecessary background noise). For example, fat may act as an undesired molecule, and will thus be filtered out from the reaction zone, in order to avoid masking or interference with the desired reaction with target molecules.

Reaction Zone (Test Line 3706), may contain an array of antibodies, antigens or enzymes according to the planned reaction for each target molecule. The reaction can either create a signal or decrease the signal, according to the nature of the reaction (competitive, sandwich or enzymatic).

According to some embodiments, Micro-array 3716 is an enlargement of test line 3706, depicting in more detail the array of antibodies, antigens or enzymes as described hereinabove.

According to some embodiments, there might be another area, referred to as Control Line 3708, that may include antibodies or proteins which indicate whether the sampling element is functional, for example, is not malfunctioned.

According to some embodiments, sampling element 3700 may include an Adhesive Backing 3714, which may be a hydrophobic layer to hold all other layers together and prevent leakage of the breastmilk sample.

According to some embodiments, sampling element 3700 may include an Adsorbent Pad 3712, made of an absorbing material, e.g., a sponge, which will soak up all the sample as it reaches the end of the sampling element.

According to some demonstrative embodiments, the sampling element may include one or more types of reactions:

Sandwich: For ELISA, this is usually performed by coating the plate with a detection antibody (which binds the target molecule), adding samples or standards to each well, then adding a capture antibody which is conjugated to a marker. This capture antibody binds to the target molecules which have already been caught by the capture antibody, and an enzyme or chemical is added to cause the color reaction (or else, the detection antibody is conjugated to gold, which when in large concentrations can be seen with the naked eye—color accumulation). The stronger the color result, the higher the concentration of the target molecule. For the sampling element of the present invention, this may be achieved by adding the detection antibody, conjugated to a marker, to Conjugate Pad 3710 and the capture antibody to Test line 3706.

Competitive reaction: Used when the target in question is too small to have an antibody pair. For ELISA, it is usually performed by coating the plate with an antibody, then adding the sample with some known concentrations of the target (Standards) that is horseradish peroxidase (“HRP”) conjugated or alike. Since the known standard is conjugated to the color and is competing on binding to the antibody, the weaker the color the higher concentration of your sample (an inverse linear equation). For the sampling element of the present invention, this may be performed by adding a known concentration of the conjugated antibody to Conjugate pad 3710 and a known concentration of the target molecule to Test line 3706. The antigens in the sample are competing with the known concentration of standards in the test line 3706 (usually made up of recombinant proteins or synthesized vitamins) on binding to the antibodies. The signal is stronger when there is a low concentration of the tested target in the sample, and vice versa. The opposite setup could also be used, in which the antibodies are present on the glass fiber membrane 3704 are unconjugated and while the known standards bound to the strip are conjugated to a marker.

According to some demonstrative embodiments, the sampling element may include an array of antibodies that can simultaneously detect different antigens, deciphering the results is done according to the exact location of each antibody on the array. For each target molecule, there may be an average of one to five, preferably three different reaction zones (triplicates). The detector will take a grey-scale photograph of the strip upon which the results of the reactions appear. The algorithm will recognize each reaction zone and give a value to each signal between 0 to 1 (the grey-scale range). Then it will give each target it's average reaction value and convert the value to a quantitative or qualitative result according to the standard graph for each target. Some targets will receive a yes or no result (e.g. presence of pathogens), others will have a threshold they will have to surpass in order to appear positive (e.g. IL-10), and the others will get a quantitative result of the tested element (e.g. 60 pg/ml of Vitamin B6).

According to some demonstrative embodiments, the device of the present invention may include a camera to photograph the results depicted upon the sampling element, and at least one computer adapted to conduct an analysis of the photograph(s) based on a pre-determined algorithm.

According to some embodiments, the algorithm may be spots detection algorithm defined to enable the calculation of the concentrations of one or more target molecules present in breastmilk, for example, to deduct regarding the nutritional and/or immunological condition of an infant which is fed with the breastmilk.

According to some embodiments, the computer of the device of the present invention may analyze a photograph and/or any suitable images of different spot's intensities as photographed of the sampling element (after reaction with a breastmilk sample).

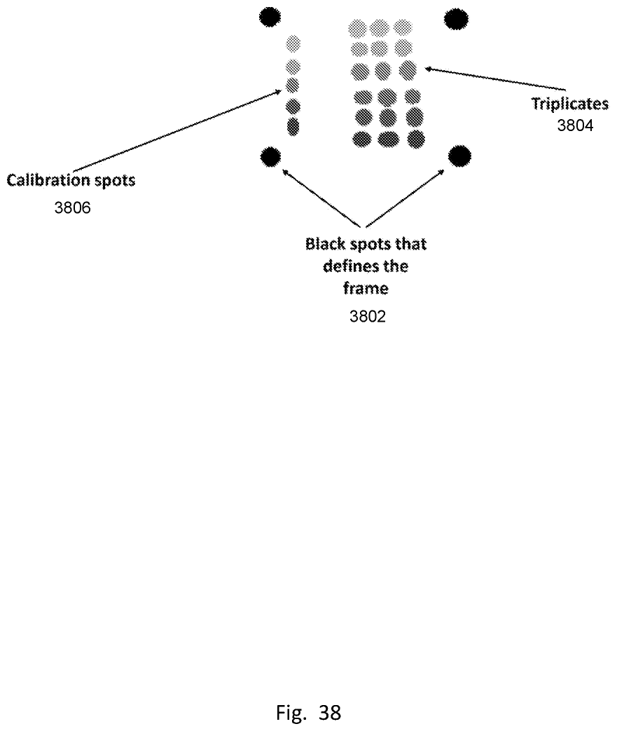

According to some embodiments, the computer may find all the spots in the image including calibration spots 3806 in the side of the image, as shown in FIG. 38. The computer of the device od the present invention may also locate the 4 black spots in the corners of the image 3802 and find the frame. According to some embodiments, black spot 3802 enable the computer to remove any spots that might be located outside the frame.

According to some demonstrative embodiments, the computer may use the intensities calibration spots 3806 and calculate the R².

According to some embodiments, the term R² as used herein, may refer to a statistical measure representing the correlation between analyzed data (e.g., from a sampling element) to a fitted regression line (for example, linear line).

According to some embodiments, the calibration of the reaction may be done by standards in different concentrations, the analysis of these standards may enable a creation of a standard curve (a linear regression line) which is highly calibrated and sensitive.

According to some embodiments, when the R² value of the regressing line is lower than a specific value, for example 0.98, it may indicate that there is a technical error in the measurement of the exact concentration of the analyte or sample.

According to some embodiments, the computer of the device of the present invention may be preconfigured to correct such an error by removing up to two standard values. Values to be removed are chosen through a statistical analysis—the algorithm may check which value seems to be farthest away from the general trend line of the standard regression. After each removal the computer may recalculate the regression equation and R² value. If after two removals the R² value is still under 0.98, the test will be considered invalid and the computer will give an error message.

According to some embodiments, the computer may check if the R² value is greater than 0.98 and may also remove maximum 2 values until it fits otherwise the computer may return an error message.

According to some demonstrative embodiments, the computer may calculate a linear equation (y=ax+b) according to the final R². The computer may then accordingly find all triplicates 3804 and calculate the average of every single triplicate of triplicates 3804. According to some embodiments, the computer may take the averages (the intensities) and calculate the concentrations according to received equation.

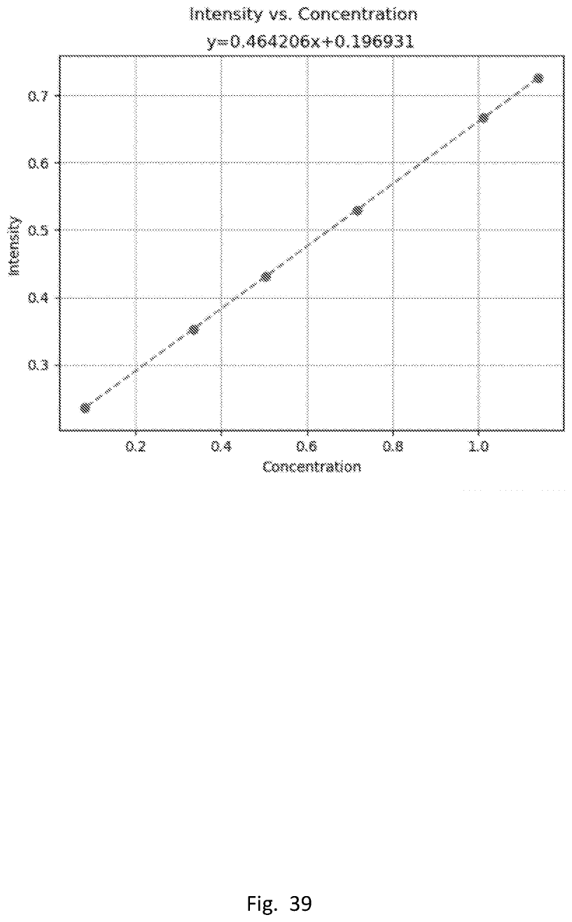

According to some embodiments, the computer may then form and/or present a graph based on intensities vs. concentrations, as demonstrated for example in FIG. 39.

### Example 20

With reference to FIG. 38, suppose the calibration concentrations are: 1.16925, 0.82970, 0.62560, 0.33515, 0.10000.

The calculated calibration intensities are: 0.705882, 0.635294, 0.494118, 0.341176, 0.235294.

The initial R² is 0.972 (smaller than the threshold 0.980) then we remove the point/points to satisfy the condition□ After removing the third index, the R² is 0.995 and the linear equation is calculated accordingly (y=0.464×+0.196). Finally, knowing the intensities of the triplicates (from the image) we could calculate their concentrations (using the linear equation). The concentrations of the triplicates are: 1.13863, 1.01191, 0.71623, 0.50503, 0.33608, 0.08264.

The actual linear equation is depicted in the graph of FIG. 39.

### Example 21

Reference is now made to FIG. 35A which depicts an image of a photo taken of a sampling element according to some demonstrative embodiments, for example, to be analyzed by the device of the present invention.

The array with the intensity, reference and indexes is presented in table 28:

TABLE 28

| X | Y | | Color |
|---|---|---|---|
| coordinate | coordinate | Reaction Zone | Intensity |
| of the | of the | number within | on a scale |
| reaction | reaction | the array | of 0-1 of |
| zone | zone | (spot number) | the spot |
| 48 | 156 | 3 | 0.74902 |
| 49 | 149 | 3 | 0.74902 |
| 49 | 150 | 3 | 0.74902 |
| 49 | 151 | 3 | 0.74902 |
| 49 | 152 | 3 | 0.74902 |
| 49 | 153 | 3 | 0.74902 |
| 49 | 154 | 3 | 0.74902 |
| 63 | 70 | 4 | 0.705882 |
| 63 | 71 | 4 | 0.705882 |
| 63 | 72 | 4 | 0.705882 |
| 63 | 73 | 4 | 0.705882 |
| 63 | 74 | 4 | 0.705882 |
| 63 | 75 | 4 | 0.705882 |

The array contains the average of every single spot in treplicator and the total average of all the Treplicator, are represented in table 29:

TABLE 29

| First | Second | Third | Average |
|---|---|---|---|
| triplicate | triplicate | triplicate | of |
| of a reaction | of a reaction | of a reaction | Triplicates |
| 0.74902 | 0.74902 | 0.74902 | 0.74902 |
| 0.705882 | 0.705882 | 0.705882 | 0.705882 |
| 0.627451 | 0.627451 | 0.627451 | 0.627451 |

### Example 22

Reference is now made to FIG. 35B which depicts an image of a photo taken of a sampling element according to some demonstrative embodiments, for example, to be analyzed by the device of the present invention.

The array with the intensity, reference and indexes is presented in table 30:

TABLE 30

| X | Y | | Color |
|---|---|---|---|
| coordinate | coordinate | Reaction Zone | Intensity |
| of the | of the | number within | on a scale |
| reaction | reaction | the array | of 0-1 of |
| zone | zone | (spot number) | the spot |
| 197 | 96 | 18 | 0.352941 |
| 197 | 97 | 18 | 0.352941 |
| 197 | 98 | 18 | 0.352941 |
| 197 | 99 | 18 | 0.352941 |
| 197 | 100 | 18 | 0.352941 |
| 198 | 153 | 18 | 0.352941 |
| 198 | 154 | 18 | 0.352941 |
| 198 | 155 | 18 | 0.352941 |
| 198 | 156 | 18 | 0.352941 |
| 198 | 157 | 18 | 0.352941 |
| 198 | 158 | 18 | 0.352941 |
| 198 | 159 | 18 | 0.352941 |

The array contains the average of every single spot in treplicator and the total average of all the Treplicator, are represented in table 31:

TABLE 31

| First | Second | Third | Average |
|---|---|---|---|
| triplicate | triplicate | triplicate | of |
| of a reaction | of a reaction | of a reaction | Triplicates |
| 0.74902 | 0.74902 | 0.74902 | 0.74902 |
| 0.705882 | 0.705882 | 0.705882 | 0.705882 |
| 0.627451 | 0.627451 | 0.627451 | 0.627451 |
| 0.54902 | 0.54902 | 0.54902 | 0.54902 |
| 0.470588 | 0.470588 | 0.470588 | 0.470588 |
| 0.352941 | 0.352941 | 0.352941 | 0.352941 |

### Example 23

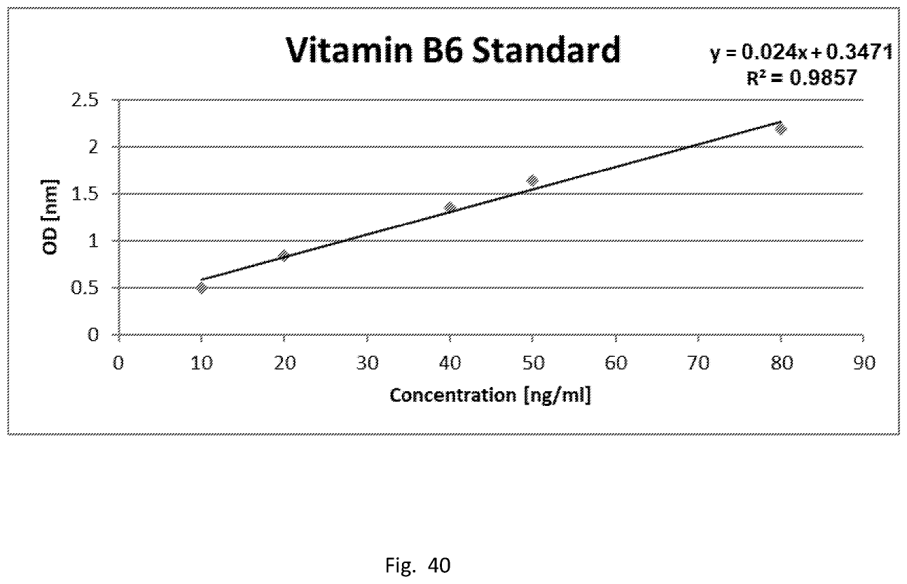

An example of Vitamin B6 standard concentration is presented in table 32 and depicted by the graph of FIG. 40:

TABLE 32
Vitamin B6

| Concentration | | |
|---|---|---|
| (ng/ml) | OD | OD-Blank |
| 10 | 0.50675 | 0.504021 |
| 20 | 0.84865 | 0.845921 |
| 40 | 1.35485 | 1.352121 |
| 50 | 1.6501 | 1.647371 |
| 80 | 2.19365 | 2.190921 |

An example of how a standard concentration curve is created through the ELISA method. Known concentrations and acquired ODs are plotted on a scatter-plot graph, from which a linear regression line and equation are extrapolated using Excel functions. The final OD used if the OD-Blank value—Blank contains all the background color which comes with the used buffers or the used standards and may add to the color intensity of the final result. In order to remove these background noises and receive a more accurate result, the Blank's OD is removed from the rest of the OD values.

### Example 24

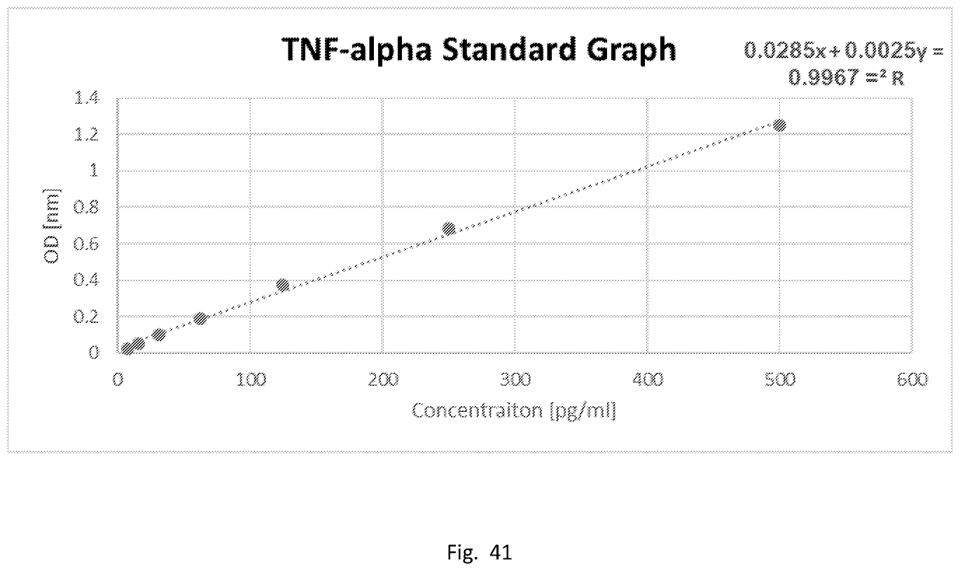

An example of TNF-alpha standard concentration is presented in table 33 and depicted by the graph of FIG. 41:

TABLE 33
TNF-alpha

| Concentration | | |
|---|---|---|
| [pg/ml] | OD | OD-Blank |
| Blank | 0.0407 | 0 |
| 7.8 | 0.062267 | 0.021567 |
| 15.6 | 0.0912 | 0.0505 |
| 31.1 | 0.140133 | 0.099433 |
| 62.5 | 0.231867 | 0.191167 |
| 125 | 0.413133 | 0.372433 |
| 250 | 0.723633 | 0.682933 |
| 500 | 1.287067 | 1.246367 |

An example of how a standard concentration curve is created through the ELISA method. Known concentrations and acquired ODs are plotted on a scatter-plot graph, from which a linear regression line and equation are extrapolated using Excel functions. The final OD used if the OD-Blank value—Blank contains all the background color which comes with the used buffers or the used standards and may add to the color intensity of the final result. In order to remove these background noises and receive a more accurate result, the Blank's OD is removed from the rest of the OD values.

### Example 25

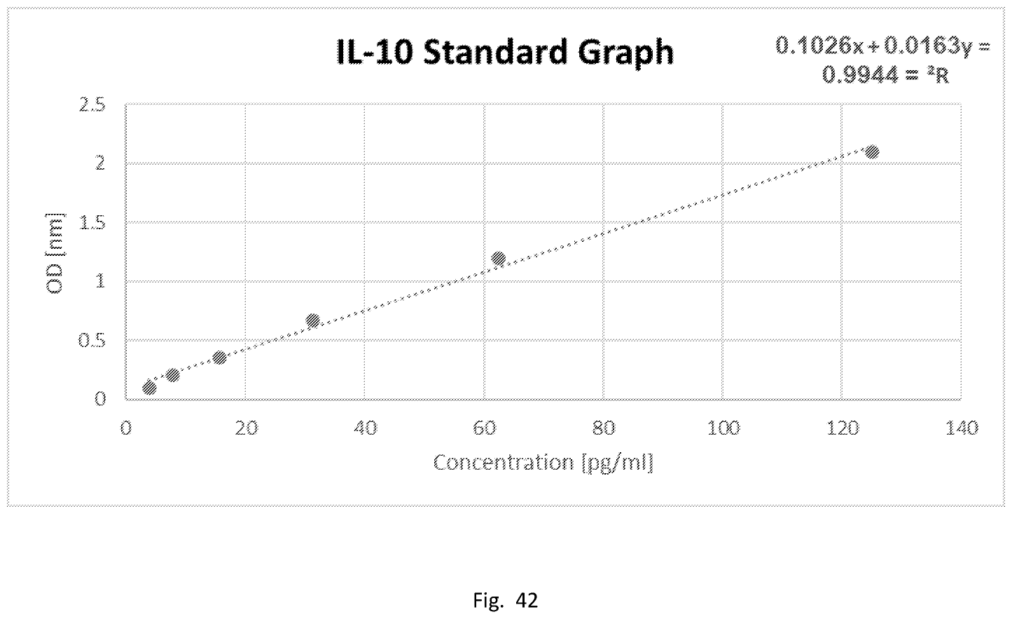

An example of Interleukin-10 (IL-10) standard concentration is presented in table 34 and depicted by the graph of FIG. 42:

TABLE 34
IL-10

| Concentration | OD | OD-Blank |
|---|---|---|
| Blank | 0.035 | 0 |
| 3.9 | 0.133833 | 0.098833 |
| 7.8 | 0.245633 | 0.210633 |
| 15.6 | 0.39395 | 0.35895 |
| 31.3 | 0.7106 | 0.6756 |
| 62.5 | 1.227933 | 1.192933 |
| 125 | 2.129633 | 2.094633 |

An example of how a standard concentration curve is created through the ELISA method. Known concentrations and acquired ODs are plotted on a scatter-plot graph, from which a linear regression line and equation are extrapolated using Excel functions. The final OD used if the OD-Blank value—Blank contains all the background color which comes with the used buffers or the used standards and may add to the color intensity of the final result. In order to remove these background noises and receive a more accurate result, the Blank's OD is removed from the rest of the OD values.

### Example 26

Exemplary Gold standards are presented in tables:

TABLE 35
Nutritional Factors

| | 5-11 | 12-30 | 1-2 | 2-4 | 4-8 |
|---|---|---|---|---|---|
| Day of Lactation | Days | Days | Months | Months | Months |
| α-lactalbumin (g/L) | 2.5-4 | 2.5-4.5 | 2.3-3.1 | 2.0-3 | 1.8-3 |
| Lactoferrin (g/L) | 2.1-4 | 1.0-2.5 | 0.8-1.8 | 0.8-1.7 | 0.7-1.7 |
| Serum albumin (g/L) | 0.3-0.6 | 0.3-0.5 | 0.3-0.5 | 0.3-0.5 | 0.3-0.5 |
| Total caseins (g/L) | 4.1-7 | 4.1-7 | 4.1-8 | 4.1-7 | 4.1-7 |

TABLE 36

| Day of | 1-5 | 6-10 | 11-20 | 21-89 | 90-180 | 181-365 |
|---|---|---|---|---|---|---|
| Lactation | Days | Days | Days | Days | Days | Days |
| Vitamin | — | 3.1-9.0 | 3.1-12 | 10.0-14.0 | 11.0-15.0 | 11.0-15.0 |
| B₁ | | | | | | |
| Vitamin | — | 25-45 | 25-45 | 25-45 | 25-45 | 25-45 |
| B₂ | | | | | | |
| Vitamin | — | 0.9-2 | 1.7-3.9 | 2.5-6.7 | 5-9.6 | 4.6-8.2 |
| B₆ | | | | | | |
| Vitamin | — | 0.2-1.2 | 0.04-0.08 | 0.03-0.07 | 0.02-0.06 | 0.02- |
| B₁₂ | | | | | | 0.06 |
| Total | 3.68- | 1.63- | 2.37-5.43 | 2.33-5.17 | 1.47-4.53 | 1.81- |
| Lipids | 4.68 | 3.91 | | | | 4.53 |
| Total | 6.68- | 6.69- | 6.69-7.51 | 6.75-7.41 | 6.94-8.28 | 6.38- |
| Carbo- | 7.58 | 7.51 | | | | 8.68 |
| hydrates | | | | | | |

TABLE 37

| | 1 | 2-3 | 3-6 | 6-9 | 9-12 | 12+ |
|---|---|---|---|---|---|---|
| | Month | Months | Months | Months | Months | Months |
| Total | 12.0- | 9.0-11.0 | 7.0-9.0 | 7.0-10.0 | 8.0-11.0 | 9.0-13.0 |
| Protein | 16.0 | | | | | |
| (ug/ul) | | | | | | |

TABLE 38

| Human Milk | Colostrum: | |
|---|---|---|
| | 20-25 | g/L |
| Oligosaccharide | Mature Milk: | |
| (HMOs) | 5/20 | g/L |

TABLE 39

| Immune Factors | | | | | | | |
|---|---|---|---|---|---|---|---|
| Breast milk | Health | | Transitional | Months | Months 4- | Months | Months |
| component | status | Colostrum | milk | 1-3 | 6 | 7-9 | 10-12 |
| Total cells | Healthy | 110 000- | 113 492- | 228 395- | 40 000- | 97 500- | 706 667- |
| per ml milk | | 2 250 000 | 883 333 | 255 769 | 588 542 | 433 333 | 1 066 667 |
| | Infection | — | 183 333 | 50 000- | 115 278- | 37 000- | 437 500- |
| | | | | 2 867 383 | 321 918 | 504 951 | 1 000 000 |
| % | Healthy | 13.2-70.4 | 0.0-1.65 | 0.07-0.45 | 0.0-1.52 | 0.0-1.09 | 0.08-0.1 |
| Leukocytes | Infection | — | 18.8 | 0.72-90.5 | 1.1-33.9 | 1.08-93.6 | >3 |
| Leukocytes | Healthy | 32 175- | 0-3450 | 160-1151 | 0-1025 | 0-1063 | 707-853 |
| per ml milk | | 784 080 | | | | | |
| | Infection | — | 34 467 | 2400- | 2164- | 1065- | >30 000 |
| | | | | 2 594 982 | 109 130 | 472 634 | |
| sIgA (μg | Healthy | 1428- | 131-1096 | 534-1276 | 257-960 | 496-1350 | 401- |
| ml−1) | | 2178 | | | | | 1044 |
| | Infection | — | 922 | 36-1418 | 652-1711 | 611-1509 | 714-789 |
| IgG (μg | Healthy | 5.3-12.2 | 2.8-9.7 | 6.4-12.4 | 4.6-10.8 | 4.0-16.4 | 5.0-16.1 |
| ml−1) | Infection | — | 13 | 6.6-17.1 | 4.8-10.1 | 5.6-14.4 | 7.6-8.8 |
| IgM (μg | Healthy | 16.2-56.1 | 8.2-29.8 | 10.6-14.9 | 6.5-11.6 | 4.2-23.7 | 8.8-23.3 |
| ml−1) | Infection | — | 10.2 | 4.5-19.8 | 10.1-15.4 | 12.6-21.8 | 14.4-19.3 |
| Lactoferrin | Healthy | 6.3-7.7 | 2.1-5.2 | 2.5-2.9 | 1.9-3.7 | 1.3-4.0 | 1.2-3.9 |
| (g l−1) | Infection | — | 4.3 | 2.9-3.7 | 2.0-3.7 | 1.6-3.3 | 1.2-3.6 |

TABLE 40

| | |
|---|---|
| CD45 | Normal: 2,122 cells/ml BM |
| | When Sick: 5,655 cells/ml BM |
| Macrophage | Normal: 300 cells/ml BM |
| | When Sick: 1,220 cells/ml BM |
| TNFα | Normal: 2.91 ± 1.51 pg/ml BM |
| | When Sick: 3.66 ± 1.68 BM |
| Total | Normal: 325 cells/ml BM |
| lymphocytes | When Sick: 2,474 cells/ml BM |
| Neutrophils | Normal: 813 cells/ml BM |
| | When Sick: 2,941 cells/ml BM |

TABLE 41

| | | |
|---|---|---|
| CMV IgM and CMV | Healthy | No antigen |
| IgG | | found |
| | Infection | Antigen Found |
| Hepatitis B Antigen | Healthy | No antigen |
| (surface antigen | | found |
| AKA HBsAg) | Infection | Antigen Found |
| Hepatitis C Antigen | Healthy | No antigen |
| (surface antigen | | found |
| AKA HBsAg) | Infection | Antigen Found |
| HIV1 Antigen (p24) | Healthy | No antigen |
| | | found |
| | Infection | Antigen Found |
| HIV2 Antigen (p24) | Healthy | No antigen |
| | | found |
| | Infection | Antigen Found |
| HTLV-I Antigen | Healthy | No antigen |
| (gp21 or p-24 Core) | | found |
| | Infection | Antigen Found |

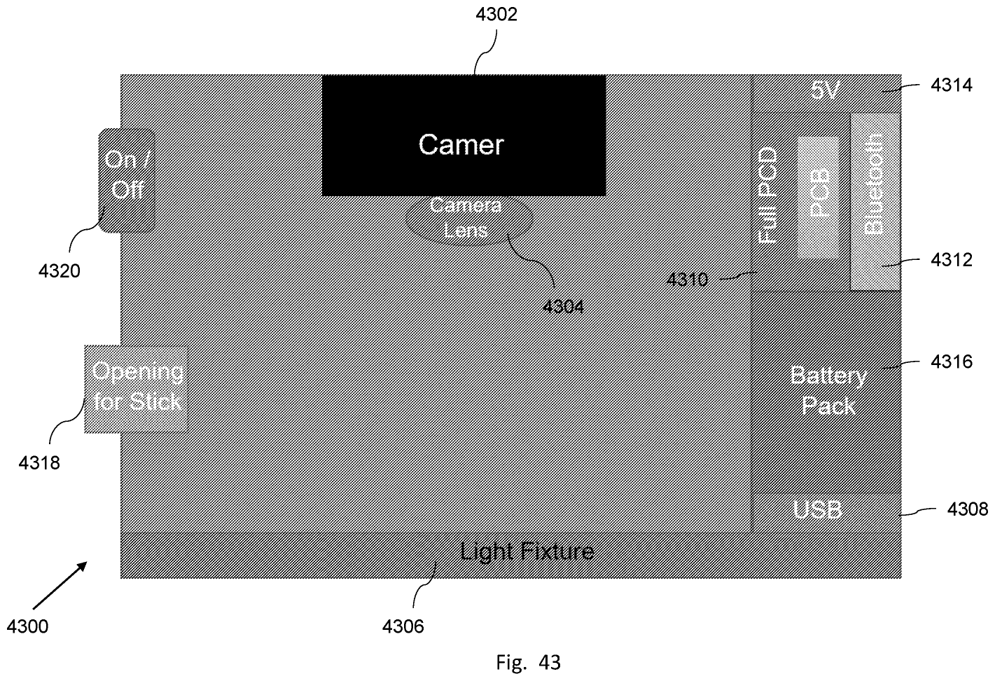

Reference is now made to FIG. 43, which illustrates an exemplary device 4300 in accordance with some demonstrative embodiments described herein.

According to some embodiments, device 4300 may include a Camera 4302. According to some embodiments, the camera may be a color camera. According to some other embodiments, the camera may preferably be a monochromatic camera.

According to some embodiments, camera 4302 may include one or more lenses 4304. According to some embodiments, one or more lens 4304 may include a wide lens, e.g., to catch the whole microarray test area.

According to some embodiments, device 4300 may include one or more lighting elements, also referred to herein as light fixture 4306. According to some embodiments, light fixture 4306 may be located below a sampling element which may be positioned within or on top of device 4300, e.g., with a white and/or reflective surface above and on the sides of device 4300.

According to some other embodiments, light fixture 4306 may be located parallel to the sampling element (for example, including one or more light bars), with reflective surfaces above and on the sides. According to some embodiments, the reflective surfaces may provide a diffused, uniform light, e.g., to enhance the contrast of spots appearing on the face of the sampling element. According to some other embodiments, light fixture 4306 may be on an upper portion of device 4300, and/or on one or both sides of the camera, e.g., with a diffuser to diffuse the light.

According to some embodiments, light fixture 4306 may include LED light fixtures.

According to some embodiments, device 4300 may include one or more USB ports 4308, e.g., to connect to an external computer.

According to some embodiments, device 4300 may include one or more computer 4310, also referred to herein as Full PCD 4310 which may include a printed circuit board (PCB): to compute image analysis. According to some embodiments, computer 4310 may include any suitable component to enable gathering, storing, analyzing and sending information, including for example: an antenna, wireless LAN, CPU, Micro SD, Memory Chip, HDMI Operating System and the like.

According to some embodiments, computer 4310 may include Bluetooth 4312, for example, to send information to a smartphone device and the like.

According to some embodiments, device 4300 may include an inside power supply 4314, for example, a 5V power supply.

According to some embodiments, device 4300 may include one or more battery Packs 4316. According to some embodiments, device 4300 may include a rechargeable using USB port or changeable (e.g. AAA).

According to some embodiments, device 4300 may include at least one opening 4318 for the sampling element.

According to some embodiments, device 4300 may include an on/off button 4320 to turn device on and off.

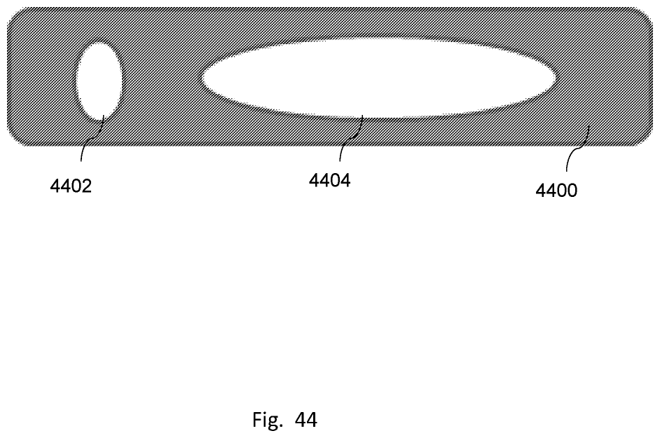

Reference is now made to FIG. 44, which illustrates an exemplary chamber 4400 for a sampling element, in accordance with some demonstrative embodiments.

As shown in FIG. 44, chamber 4400 may include one or more openings to encompass a sampling element according to some demonstrative embodiments of the present invention.

According to some embodiments, chamber 4400 may include one or more openings 4402, to introduce a sample of breastmilk.

According to some embodiments, chamber 4400 may include one or more windows 4404, wherein window 4404 may be an opening through which the reaction results of the sampling element are visible. According to some embodiments, specifically, the test zone and control zone are visible through window 4404 when the sampling element is encompassed within chamber 4400.

While this invention has been described in terms of some specific examples, many modifications and variations are possible. It is therefore understood that within the scope of the appended claims, the invention may be realized otherwise than as specifically described.

1. A device for the analysis of breastmilk, comprising:
at least one camera to photograph a sampling element, wherein said sampling element comprises one or more arrays of reagents reacting with one or more target molecules present in a sample of said breastmilk and presenting one or more arrays with a plurality of spots comprising one or more intensity levels, and wherein said camera is adapted to photograph said plurality of spots;
at least one computer adapted to analyze said one or more intensity levels of said spots; and
at least one output component for presenting results of said analysis and for providing a recommendation relating to nutritional or immunological needs of an infant fed by said breastmilk.

2. The device of claim 1, wherein said camera is selected from a group comprising: a monochromatic camera, a fluorescent camera or a color camera.

3. The device of claim 1, wherein said computer comprises a pre-defined algorithm for the comparison of said results with other data.

4. The device of claim 4, wherein said other data is selected from the group including: previous results obtained by said user of said device, gold standard values, average values or ranges of target molecules as presented in the relevant literature.

5. The device of claim 1, wherein said recommendation includes one or more suggestions to change the diet of said infant.

6. The device of claim 1, wherein said recommendation may include one or more indications regarding a status of infection or disease in said infant.

7. The device of claim 1, wherein said analysis comprises a determination of the amount or concentration of macromolecules or nutrients in said breastmilk.

8. The device of claim 7, wherein said macromolecules or nutrients are selected from the group including: Vitamins, Human Milk Oligosaccharides (HMO), long chain polyunsaturated fatty acids (LCPUFA) and Total Proteins.

9. The device of claim 1, wherein said analysis comprises an immunological analysis comprising determination of the amount or concentration of at least one immunological factor in said breastmilk selected from the group including: Lactoferrin, Leukocytes and Immunoglobulins.

10. The device of claim 9, wherein said at least one immunological factor is Lymphocyte Common Antigen (CD45).

11. The device of claim 9, wherein said at least one immunological factor is sIgA.

12. A system for the analysis of breastmilk, comprising
a lateral flow sampling element comprising a plurality of spots comprising a microarray of molecules selected from the group including antibodies, proteins and vitamins;
a device comprising
at least one camera to photograph a color intensity level of said plurality of spots of said sampling element;
at least one computer adapted to analyze said color intensity level of said plurality of spots; and
at least one output component for presenting results of said analysis and for providing a recommendation relating to nutritional or immunological needs of an infant fed by said breastmilk or of a mother breastfeeding said breastmilk.

13. Use of a device of claim 1 for providing at least one recommendation regarding dietary or immunological needs of an infant breast fed by said breastmilk.
